(19) United States
(12) Patent Application Publication
(10) Pub. No.: US 2023/0338519 A1
(43) Pub. Date: Oct. 26, 2023

(54) FUSION PROTEIN WITH IMMUNOENHANCING ACTIVITY

(71) Applicant: Transmed Gothenburg AB, Gothenburg (SE)

(72) Inventors: Nils Lycke, Västra Frölunda (SE); Mohammad Arabpour, Västra Frölunda (SE); Fredrik Höök, Alingsås (SE)

(21) Appl. No.: 17/998,693

(22) PCT Filed: May 14, 2021
(86) PCT No.: PCT/SE2021/050460

§ 371 Date: Nov. 14, 2022

Related U.S. Application Data
(60) Provisional application No. 63/025,550, filed on May 15, 2020.

(51) Int. Cl. A61K 39/39; C07K 16/28; C07K 14/28; A61P 37/04; A61K 39/145

(52) U.S. Cl. CPC A61K 39/39; C07K 16/2839; C07K 14/28; A61P 37/04; A61K 39/145; C07K 2317/622; C07K 2319/55; A61K 2039/505

(57) ABSTRACT

The present invention relates to a fusion protein, a nucleotide sequence encoding such a fusion protein, the use thereof as an adjuvant or vaccine. The fustin protein comprises a bacterial exotoxin and a single chain antibody fragment (scFv) that specifically binds to a surface marker on dendritic cells. The fusion protein is advantageously administered intranasally, orally or intrapulmonarily.

### TECHNICAL FIELD

The present invention generally relates to nucleotide sequences coding for fusion proteins with immunoenhancing activity, such fusions proteins and uses thereof as adjuvants or vaccines.

### BACKGROUND

Most commercial vaccines are injected and consist of live attenuated or killed whole organisms. However, a more rational design of future vaccines may need to consider which components to include, the formulation, the route of delivery and the immunoenhancer or adjuvant to use. For example, the benefit of mucosal vaccination as opposed to injected vaccines is that local immunoglobulin A (IgA) antibodies and tissue resident memory T cells are more effectively induced. This can be absolutely critical for stimulating immune protection against infections as has been observed for cholera, tuberculosis, rotavirus and influenza virus vaccines. The use of customized proteins in subunit or conjugate vaccines allow for increased control over the vaccine formulation and the elicited immune response. However, a potential problem with mucosal vaccines is that they require relatively large amounts of antigen and effective, safe and non-toxic mucosal adjuvants.

This has prompted research with the aim of identifying mucosal adjuvants that could find general use in mucosal vaccines. A powerful mucosal adjuvant should be non-toxic and greatly improve immunogenicity and generate immunological memory to the vaccine antigens. Most antigens are normally poor immunogens when administered perorally or intranasally and this is why adjuvants are needed. Two principally different approaches have been taken to improve immunogenicity of mucosally delivered vaccine antigens. The first has focused on constructing powerful delivery systems for oral antigens, such as encapsulated microparticles, nanoparticles or immune stimulating complexes (iscomes), such as disclosed in U.S. Pat. No. 7,452,982. This U.S. patent discloses a immunogenic complex comprising at least one glycoside and at least one lipid, integrated into an iscom complex or matrix, and at least one antigen that is integrated into the iscom complex or coupled on to or mixed with the iscom complex or iscom matrix complex. The second approach is to construct an adjuvant that will modulate and greatly augment the immune response to the mucosally delivered vaccine antigen. This results in enhanced vaccine-specific IgA and T cell immunity and the development of immunological memory at mucosal membranes and systemically in lymph nodes and the spleen. U.S. Pat. No. 5,917,026 discloses a deoxyribonucleic acid (DNA) sequence encoding for a fusion protein capable of augmenting the immune response to admixed vaccine antigens and thereby useful as an adjuvant protein. The DNA sequence comprises (i) a first DNA sequence encoding the A1 subunit of a bacterial enterotoxin selected from the group consisting of cholera toxin (CT) and Escherichia coli heat labile enterotoxin (LT) and (ii) a second DNA sequence encoding for a peptide, which specifically binds to a receptor expressed on an antigen presenting cell (APC) expressing major histocompatibility complex (MHC) class I or class II antigen. The APCs are selected from the group consisting of lymphocytes, macrophages, dendritic cells, Langerhans cells, epithelial cells and other potential cells. The fusion protein is water-soluble and specifically binds to a cell receptor recognized by the peptide encoded by the second DNA sequence. The fusion protein is internalized by an APC expressing the receptor or that can bind the adjuvant in some other way.

There is still a need for improving the adjuvant effect and in particular in mucosal adjuvants used in connection with mucosal vaccination.

### SUMMARY

It is a general objective to provide improved adjuvant and/or vaccination effects, in particular, in connection with mucosal vaccination.

This and other objectives are met by the embodiments as disclosed herein.

The invention is defined in the independent claims. Further embodiments of the invention are defined in dependent claims.

An aspect of the invention relates to a nucleotide sequence encoding a fusion protein. The nucleotide sequence comprises a first nucleotide sequence encoding a bacterial exotoxin and a second nucleotide sequence encoding a single chain antibody fragment (scFv) that specifically binds to a surface marker on antigen presenting cells.

The invention also relates to a nucleotide sequence according to above for use as a medicant, for use as a vaccine adjuvant or, if the nucleotide sequence comprises a third nucleotide sequence encoding at least one virus or bacterial epitope, for use as a vaccine.

The invention further relates to an expression vector which comprises the nucleotide sequence according to above and a cell comprising the nucleotide sequence according to above and/or the expression vector.

Another aspect of the invention relates to a fusion protein comprising a bacterial exotoxin and a scFv that specifically binds to a surface marker on antigen presenting cells.

The invention also relates to an adjuvant composition comprising the fusion protein according to above and a pharmaceutically acceptable carrier and a vaccine composition comprising a fusion protein comprising the bacterial exotoxin, the scFv that specifically binds to a surface marker on antigen presenting cells and at least one virus or bacterial epitope in a pharmaceutically acceptable carrier.

Further aspects of the invention relates to a fusion protein according to above for use as a medicament, for use as an adjuvant or for use as a vaccine.

An aspect of the invention also relates a method of vaccinating a subject against a viral or bacterial infection or disease. The method comprises administering a vaccine composition according to the invention to the subject.

The fusion protein of the embodiments have immunoenhancing activity and is useful as adjuvant in a vaccine composition or indeed as a vaccine by itself. The fusion protein is in particular suitable as mucosal adjuvants and in mucosal vaccination.

### BRIEF DESCRIPTION OF THE DRAWINGS

The embodiments, together with further objects and advantages thereof, may best be understood by making reference to the following description taken together with the accompanying drawings, in which:

FIG. 1 A) Schematic representation of in silico cloning strategy of the adjuvant active fusion proteins; CTA1-3M2e-3Eα-SIINFEKL-P323-CD103-Flag-His in prokaryoutic (E. coli) expression system and CTA1-3M2e-3Eα-CD103-Flag-His in eukaryoutic system (Baculovirus). B) Expressed and purified protein SDS-PAGE, densogram and correspondent Westen blot by anti-FLAG staining. C) Alignment from the CTA1 in CTA1-DD vs CTA1-CD103 constructs. D) Western blot analysis of the CTA1-II-DD (1), CTA1(R7K)-II-DD (2), CTA1-II-CD103 (3) and CTA(R7K)—II-CD103 (4) molecules. E) The ADP-ribosylating activity of the CTA1-enzyme in the respective fusion protein as assessed by the agmatine in vitro test and the failure to ADP-ribosylate when using the mutant CTA1R9K-fusion proteins.

FIG. 2 A) A comparative analysis of the adjuvant ability of the CTA1-II-DD and CTA1-II-CD103-fusion proteins admixed with 5 μg NP-CGG and/or TT. Specific antibody responses were determined by ELISA and values are given in log₁₀-titers±SD of indicated isotypes in A) IgA in bronchoalveolar lavage (BAL) and B) IgG2c in serum or C) total IgG in serum. Statistical significance is given as p-values: * p<0.05, **p<0.01, ***p<0.001, ****p<0.0001.

FIG. 3 A) A comparison of the ability of CTA1-I/II-DD and CTA1-I/II-CD103 for enhancing CD4 T cell priming in C57Bl/6 mice (CD45.2+). After adoptive transfer of CFSE-labeled CD45.1+ OT-II TCR Tg CD4 T cells i.v. the mice were immunized i.n. with 5 μM OVA (an equimolar dose to the p323 dose), CTA1-I/II-DD or CTA1-I/II-CD103 the next day. On day 5 mice were analyzed for OT-II CD4 T cell proliferation in the draining mLN (dilution opf CFSE-label by FACS) and the frequency of the expanding OT-II cells was assessed in the different groups (mean±SD of 5 mice). B) Evaluation of the longevity of the priming ability of DCs in the mLN: The experimental protocol was the same as in A, except that CFSE-labeled OT-II CD4 T cells were adoptively transferred i.v. on days 1 or 4 after immunization with the fusion proteins. The OT-II CD4 T cell response (CFSE-dilution) was evaluated and given as a % of cells in division as of all OT-II cells. C) Evaluation of the CD4 T cell subset differentiation resulting from the use of the DD or CD103-targeting adjuvant given i.n. according to protocol used in A. The CD4 T cell responses were determined by the ELISPOT test (IMMUNOSPOT) and the single cell production of IFNγ (dots marked with arrows) or IL-17 (unmarked dots) was analyzed and figures are given as SFC/10⁵ OT-II cells. D) Assessment of the role of the CTA1-enzyme for the augmenting effect of the fusion proteins. The same protocol as in A was used and the OT-II proliferation was assessed by dilution of CFSE. CD4 T cell expansion in the draining mLN was not observed with a mutant, inactive, CTA1-moiety (values are given as mean %±SD of all CD4 T cells (left panel) or as responding OT-II cells (right panel)); p-values *p<0.05, **p<0.01, ***p<0.001, ****p<0.0001.

FIG. 4 A) The cell binding efficiency of the scFv CD103 fusion protein was determined in various subsets of dendritic cells (DCs). The DC-target populations were defined in migrating DCs (MHC II^{high} CD11c^{high}) isolated from the mLN of CD103^{-/-} (no CD103 on DCs) or WT mice. Flow cytometry and specific monoclonal antibodies (mAbs) were used to detect the DC-subsets and anti-FLAG mAb to detect the fusion proteins. scFvCD103-fusion proteins failed to bind to any DC-subsets in CD103^{-/-} cells while DD-fusion proteins bound all DC subsets. B) The conditions were similar to that in A, but migratory DCs from wild-type mLN exhibited different DC-subsets as defined by CD103⁺CD11b⁻, CD103⁺CD11b⁺ or CD103-CD11b⁺ markers. The selective binding of scFvCD103 fusion proteins to CD103⁺ DCs was convincingly shown, while DD bound all three DC-subsets.C) Investigations of the priming ability of in vivo targeted DC subsets following i.n. immunizations with the CTA1-CD103 adjuvant. Mice with different restrictions in their DC-subset repertoire (lacking certain DC subsets) were immunized i.n. following adoptive transfer of CFSE-labelled OT-II cells (CD45.1+). We compared the response in WT C57BL/6, CD103^{-/-} or BATF3^{-/-} (no cDC1 cells) mice (5 μM) CTA1-II-DD or CTA1-II-CD103. After 24 h migratory DCs (MHC^{high}, CD11c^{high}) and OT-II cells (after 4 days) were isolated and analyzed by flow cytometry. The mean frequency of cDC1 (XCR1+), CD11b⁺ (CD103⁻), CD103⁺ (CD11b⁻) and DP (CD103⁺CD11b⁺) DCs is given as % DC subset of 5 mice in each group (left panel). OT-II CD4 T cell proliferation (right panels) was found to depend on the presence of cDC1 cells (missing in Batf3^{-/-}) following i.n. CD103-adjuvant immunizations while this dependency was not found with DD constructs. The polarization into Th17 differentiation (Lower panel) was seen with freshly isolated cDC1 cells from mLN in mice after they had been i.n. immunized with CD103-fusion proteins from mLN from WT C57BL/6 mice. Statistical significance; student t-test (A,B) or ANOVA with Dunnett's posttest (C,D). Statistical significance; *p<0.05, **p<0.01, ***p<0.001, ****p<0.0001.

FIG. 5 A) The augmented capacity of scFvCD103-targeted CTA1-CD103 fusion protein for induction of immune protection against a live influenza A virus infection. A comparisons of immune protective capacity was determined by CTA1-3M2e-CD103 and CTA1-3M2e-DD. Balb/c mice were immunized i.n.×3 with the respective fusion protein at 5 μM per dose and immune protection against a challenge infection was assessed 3 weeks after the final immunization with a 4×LD₅₀ dose of a mouse adapted PR8 influenza A virus strain. Percentage of surviving animals, weight loss and lung viral titer in Plaque Forming Unit (PFU) are shown. B) Representative FACS plots of M2e-tetramer-specific lung CD4 T cells isolated from immunized or naïve control mice after challenge infection as indicated (lower panels). Analysis of the frequency of resident memory rorγt+ M2e-specific CD4 T cells (Th17 cells) in the lungs. ANOVA with Dunnett's T3 post-test analysis: ns; not significant, p-values *p<0.05, **p<0.01, ***p<0.001, ****p<0.0001.

FIG. 6 A) CD103-targeted adjuvants explore the cross-presentation ability of cDC1 cells to prime CD8 T cells in vivo. An adoptive transfer model was used with CFSE-labeled CD45.1+ OT-1 TCR Tg donor CD8 T cells injected into C57Bl/6 (CD45.2+) recipient mice. CD8 T cell priming effects of i.n. immunization with ovalbumin (OVA), CTA1-1-II-DD or CTA1-1-II-CD103 was assessed by FACS. Values are given in % and SD of proliferating OT-1 cells. B) Comparison between CD103 and DD constructs for their ability to stimulate cross-presentation and priming of SIINFEKL-specific IFNγ-ELISPOTs OT-1 cell. At 5 days following the i.n. immunizations with the fusion proteins in the adoptive transfer model. C) Cytotoxic lymphocytes (CTLs) were stimulated by i.n. immunization of C57BL6 mice with a 5 μM dose of OVA, CTA1-II-DD, CTA1-1-II-DD or CTA1-1-II-CD103 fusion protein. One week later mice were injected i.v. with CFSE-labelled splenocyts pulsed with 1 μM SIINFEKL peptide (Target cells) or Far Red-labelled unpulsed control splenocytes (Bystander cells). At 20 h after the i.v. injection of the target cells the mLN was analyzed for CTL activity. D) A prophylactic cancer vaccine based on the scFvCD103 targeting was evaluated showing protection against a metastatic melanoma cancer (B16F1OVA cell line). C57Bl/6 mice were immunized i.n.×2 with CTA1-1-II-CD103 or CTA1-1-II-DD fusion proteins at 5 μM per dose. At day 7 post immunization mice received 100,000 B16F1OVA cells i.v and kept for 4 weeks. Lungs were inspected for cancer metastasis (black metastatic) dots on their surface. Cancer therapeutic vaccines evaluation done by transferring 100,000 B16F1OVA into C57Bl/6 mice cDC1 cells isolated and assessing the tumor growth surface area and survival over time was monitored. Statistical significance was calculated using ANOVA with Dunnett's post-test (A,B,C) or Student's t-test (D). ns; not significant; p-values *p<0.05, **p<0.01, ***p<0.001, ****p<0.0001.

FIG. 7. A) Molecular structure representation of the lipids used in Example 7: 1,2-dioleoyl-sn-glycero-3-phosphocholine (DOPC) and 1,2-distearoyl-sn-glycero-3-phosphocholine (DSPC). B) schematicrepresentation of the different vaccine formulations used in this study; liposomes with covalently coupled FP (DOPC-PEG-FP and DSPC-PEG-FP) and free FP admixed with liposomes (DOPC-PEG+FP and DSPC-PEG+FP).

FIG. 8. Size distributions of the different particle formulations (see figure labels) presented in Table 1 as determined by NTA.

FIG. 9. A) Gating strategy used for the antigen presentation assay from left to right: i) cell debris was excluded in the SSC vs FSC plot; ii) gating for 7AAD negative cells excludes dead cells; iii) cells positive for dendritic cell markers MHC II and CD11c were selected. B) Representative YAe histograms for free FP, DOPC-PEG-FP and DSPC-PEG-FP. C) Median fluorescence intensity of labelled YAe as a function of time as measured by flow cytometry. The data was normalized to zero at the beginning of the experiment and to unity at the value of the free FP at 24 h. D) YAe median fluorescence intensity after 24 hours. Error bars show standard error of the mean. Statistical significance: *p<0.05, **p<0.01, ***p<0.001.

FIG. 10. A) Normalized MHC II median fluorescence intensity ratio after 24 h. B) YAe to MHC II median fluorescence intensity ratio. Error bars show standard error of the mean. Statistical significance: *p<0.05, **p<0.01, ***p<0.001.

FIG. 11. A) Representative CD86 and CD80 histograms of the YAe+ population for the different formulations. B) Change in CD86 median fluoresence intensity at 24 h compared to free FP. C) Change in CD80 median fluoresence intensity at 24 h compared to free FP. In C and D the median fluorescence intensity was normalized to zero for unstimulated cells and to unity at the value of the free fusion protein. Error bars show standard error of the mean. Statistical significance: *p<0.05, **p<0.01, ***p<0.001.

FIG. 12. Quantification by ELISA of released cytokines in supernatant following activation with vaccine formulations for 24 h. Cytokines investigated were A) IL-6 and B) IL-1p. Error bars show standard error of the mean. Statistical significance: *p<0.05, **p<0.01, ***p<0.001.

### DETAILED DESCRIPTION

The present invention generally relates to nucleotide sequences coding for fusion proteins with immunoenhancing or immunopotentiating activity, such fusions proteins and uses thereof as adjuvants or vaccines.

The present invention relates to fusion proteins useful as adjuvants or vaccines, and in particular in connection with mucosal vaccination. Such mucosal vaccination has the advantage over traditional injected vaccines in that local IgA antibodies and tissue resident memory CD4 and CD8 T cells are more effectively induced. For instance, intranasal (i.n.) vaccines against influenza virus infections are superior to intramuscular injections of the vaccines due to dramatically augmented effect on induction of IgA and lung tissue resident memory T cells.

Mucosal vaccines require relatively large amounts of antigen in order to be effective. This can at least partly be solved by using effective, safe and non-toxic mucosal adjuvants. The present invention relates to a nucleotide sequence encoding a fusion protein and such a fusion protein that can be used as adjuvant, such as for mucosal vaccines, i.e., an adjuvant protein. The fusion protein comprises an adjuvant active component that is a powerful immunoenhancer that is safe and non-toxic and acts on dendritic cells (DCs) as well as follicular dendritic cells (FDCs), greatly augmenting CD4 and CD8 T cell responses, the germinal center (GC) reaction and memory B cell and plasma cell development. The fusion protein also comprises a cell-binding component that targets the fusion protein and vaccine to cell subsets known to prime CD4 and CD8 T cells in the draining lymph node. This cell-binding component thereby significantly improves the adjuvant or vaccine specificity, which thereby improves its effectiveness even at very low concentrations. In particular, the cell-binding component targets a distinct subset or subsets of antigen presenting cells (APCs), preferably DCs, that is or are key to priming and stimulating effector T cells, including tissue resident memory T cells, in the draining lymph nodes.

An aspect of the invention relates to a nucleotide sequence encoding a fusion protein. The nucleotide sequence comprises a first nucleotide sequence encoding a bacterial exotoxin and a second nucleotide sequence encoding a single chain antibody fragment (scFv) that specifically binds to antigen presenting cells (APCs).

The nucleotide sequence is typically in the form of a deoxyribonucleic acid (DNA) sequence. However, the nucleotide sequence may alternatively be in the form a ribonucleic acid (RNA) sequence, such as a messenger RNA (mRNA) sequence.

The nucleotide sequence comprises at least two nucleotide sequences, also referred to as subsequences, encoding the bacterial exotoxin and the scFv, respectively.

There is a group of bacterial exotoxins that exert strong enzymatic activity in mammalian cells. These exotoxins include bacterial enteroxins and pertussis toxin.

These bacterial exotoxins, including the cholera toxin (CT) and Escherichia coli heat labile toxin (LT), act by adenosine diphosphate (ADP) ribosylation of guanosine triphosphate (GTP) binding proteins in the cell membrane of the target cells. In the ADP-ribosylation reaction, nicotinamide adenine dinucleotide (NAD⁺) is split into free nicotinamide and an ADP-ribose moiety, which is associated with the guanidinium group of an arginine in the α subunit (αₛ) of the stimulatory G protein (Gₛ). The Gₛ protein becomes permanently activated and stimulates adenylate cyclase, which results in the formation of large quantities of intracellular cyclic adenosine monophosphate (cAMP). The increase in cAMP may then act to immunomodulate many diverse immune reactions, such as increasing B lymphocyte differentiation, augmenting co-stimulation of APCs, inhibiting or promoting various T cell functions and/or modulating apoptosis in lymphoid cells. The mechanism by which cAMP exerts its function is pleiotropic and increases in intracellular cAMP may affect different cells in different ways. Also, cells of a particular cell lineage may respond with inhibited function to cAMP at one stage of differentiation whereas at a different stage strong enhancing effects may be observed. The effect may also be independent on cAMP. The bacterial exotoxins also augment effects on liver endosomal processing with enhanced acidification due to an enzymatic effect on the H⁺ pump. In fact, these bacterial exotoxins have been shown to increase ATP-dependent steady-state intravascular H⁺ concentrations by up to several 100%. The mechanism is unlikely to reflect major changes in vesicle ion transporters but rather indicate an increase in the number of H⁺ pumps per endosome and/or changes in fusion, remodeling, and maturation of early endocytic vesicles in response to cAMP. The bacterial exotoxins may also cause a redistribution or activation of vacuolar H⁺ pumps, such that toxin-treated endosome membranes contain a greater number (or density) of active pumps without other changes in vesicle geometry. This could lead to an increased function of the endosomal machinery for protein degradation, typically augmenting APC function and co-stimulation, in particular in DCs, but also in other APCs.

CT is composed of five enzymatically inactive, non-toxic B subunits (CTB) held together in a pentamere structure surrounding a single A subunit that contains a linker to the pentamere via the A2 subunit or fragment (CTA2) and the toxic enzymatically active A1 subunit or fragment (CTA1) of the molecule. The toxic CTA1 has strong ADP-ribosyl transferase activity and is thought to act on several G-proteins, of which the activity is strongest on Gₛ. This results in activation of adenylate cyclase and the subsequent intracellular increase in cAMP. CTB binds to the ganglioside monosialotetrahexosylgangliosid (GM1) receptor, present on most mammalian cells, including lymphocytes and gut epithelial cells, and CTA is thereafter translocated into the cell-membrane/cytosol of the cell where the CTA1 and CTA2 are dissociated. The profuse diarrheal response in cholera is thought to result from CTA1-induced increased cAMP levels in the intestinal epithelium with subsequent loss of electrolytes and water.

In an embodiment, the bacterial exotoxin is selected from a group consisting of an A1 subunit of a bacterial enterotoxin and a pertussis toxin.

The bacterial enterotoxin is preferably selected from the group consisting of cholera toxin (CT) and Escherichia coli heat labile enterotoxin (LT). The pertussis toxin is preferably the pertussis toxin subunit S1.

Pertussis toxin (PT) is a protein-based AB₅-type exotoxin produced by the bacterium Bordetella pertussis, which causes whooping cough. PT is released from B. pertussis in an inactive form. Following PT binding to a cell membrane receptor, it is taken up in an endosome, after which it undergoes retrograde transport to the trans-Golgi network and endoplasmic reticulum. At some point during this transport, the A subunit (S1 subunit) becomes activated. In fact, PT is known to dissociate into two parts in the endoplasmic reticulum, the enzymatically active A subunit (S1 subunit) and the cell-binding B subunit. PT catalyzes the ADP-ribosylation of the αᵢ subunits of the heterotrimeric G protein. This prevents the G proteins from interacting with G protein-coupled receptors on the cell membrane, thus interfering with intracellular communication. The Gi subunits remain locked in their GDP-bound, inactive state, thus unable to inhibit adenylate cyclase activity, leading to increased intracellular concentrations of cAMP, which interfers with normal cellular functions, including receptor-mediated signaling.

In an embodiment, the first nucleotide sequence lacks a nucleotide sequence encoding any subunit of the bacterial enterotoxin besides the A1 subunit if the bacterial exotoxin is the A1 subunit of a bacterial enterotoxin. Correspondingly, if the bacterial exotoxin is the S1 subunit of a PT, the first nucleotide sequence preferably lacks a nucleotide sequence encoding any subunit of the PT besides the S1 subunit.

In an embodiment, the first nucleotide sequence encodes CTA1. In a particular embodiment, the first nucleotide sequence encoding CTA1 comprises, preferably consists of, SEQ ID NO: 1:

ATGGATGATAAGTTATATCGGGCAGATTCTAGACCTCCTGATGAAATAAA
GCAGTCAGGTGGTCTTATGCCAAGAGGACAGAGTGAGTACTTTGACCGAG
GTACTCAAATGAATATCAACCTTTATGATCATGCAAGAGGAACTCAGACG
GGATTTGTTAGGCACGATGATGGATATGTTTCCACCTCAATTAGTTTGAG
AAGTGCCCACTTAGTGGGTCAAACTATATTGTCTGGTCATTCTACTTATT
ATATATATGTTATAGCCACTGCACCCAACATGTTTAACGTTAATGATGTA
TTAGGGGCATACAGTCCTCATCCAGATGAACAAGAAGTTTCTGCTTTAGG
TGGGATTCCATACTCCCAAATATATGGATGGTATCGAGTTCATTTTGGGG
TGCTTGATGAACAATTACATCGTAATAGGGGCTACAGAGATAGATATTAC
AGTAACTTAGATATTGCTCCAGCAGCAGATGGTTATGGATTGGCAGGTTT
CCCTCCGGAGCATAGAGCTTGGAGGGAAGAGCCGTGGATTCATCATGCAC
CGCCGGGTTGTGGGAATGCTCCAAGATCATCGGGATCTACTA

In another embodiment, the first nucleotide sequence encodes the A1 subunit of E. coli LT (LTA1). In a particular embodiment, the first nucleotide sequence encoding LTA1 comprises, preferably consists of, SEQ ID NO: 2:

ATGAAGAACATCACCTTCATCTTCTTCATCCTGCTGGCCAGCCCCCTGTA
CGCCAACGGCGACAAGCTGTACAGGGCCGACAGCAGGCCCCCCGACGAGA
TCAAGCACAGCGGCGGCCTGATGCCCAGGGGCCACAACGAGTACTTCGAC
AGGGGCACCCAGATCAACATCAACCTGTACGACCACGCCAGGGGCACCCA
GACCGGCTTCGTGAGGTACGACGACGGCTACGTGAGCACCAGCCTGAGCC
TGAGGAGCGCCCACCTGGCCGGCCAGAGCATCCTGAGCGGCTACAGCACC
TACTACATCTACGTGATCGCCACCGCCCCCAACATGTTCAACGTGAACGA
CGTGCTGGGCGTGTACAGCCCCCACCCCTACGAGCAGGAGGTGAGCGCCC
TGGGCGGCATCCCCTACAGCCAGATCTACGGCTGGTACAGGGTGAACTTC
GGCGTGATCGACGAGAGGCTGCACAGGAACAGGGAGTACAGGGACAGGTA
CTACAGGAACCTGAACATCGCCCCCGCCGAGGACGGCTACAGGCTGGCCG
GCTTCCCCCCCGACCACCAGGCCTGGAGGGAGGAGCCCTGGATCCACCAC
GCCCCCCAGGGCTGCGGCAACAGCAGCAGGACCATCACCGGCGACACCTG
CAACGAGGAGACCCAGAACCTGAGCACCATCTACCTGAGGAAGTACCAGA
GCAAGGTGAAGAGGCAGATCTTCAGCGACTACCAGAGCGAGGTGGACATC
TACAACAGGATCAGGGACGAGCTG

In a further embodiment, the first nucleotide sequence encodes the PT S1 subunit (PTS1). In a particular embodiment, the first nucleotide sequence encoding PTS1 comprises, preferably consists of, SEQ ID NO: 3:

GACGATCCTCCCGCCACCGTATACCGCTATGACTCCCGCCCGCCGGAGGA
CGTTTTCCAGAACGGATTCACGGCGTGGGGAAACAACGACAATGTGCTCG
ACCATCTGACCGGACGTTCCTGCCAGGTCGGCAGCAGCAACAGCGCTTTC
GTCTCCACCAGCAGCAGCCGGCGCTATACCGAGGTCTATCTCGAACATCG
CATGCAGGAAGCGGTCGAGGCCGAACGCGCCGGCAGGGGCACCGGCCACT
TCATCGGCTACATCTACGAAGTCCGCGCCGACAACAATTTCTACGGCGCC
GCCAGCTCGTACTTCGAATACGTCGACACTTATGGCGACAATGCCGGCCG
TATCCTCGCCGGCGCGCTGGCCACCTACCAGAGCGAATATCTGGCACACC
GGCGCATTCCGCCCGAAAACATCCGCAGGGTAACGCGGGTCTATCACAAC
GGCATCACCGGCGAGACCACGACCACGGAGTATTCCAACGCTCGCTACGT
CAGCCAGCAGACTCGCGCCAATCCCAACCCCTACACATCGCGAAGGTCCG
TAGCGTCGATCGTCGGCACATTGGTGCGCATGGCGCCGGTGATAGGCGCT
TGCATGGCGCGGCAGGCCGAAAGCTCCGAGGCCATGGCAGCCTGGTCCGA
ACGCGCCGGCGAGGCGATGGTTCTCGTGTACTACGAAAGCATCGCGTATT
CGTTC

In an embodiment, the first nucleotide sequence encodes multiple, i.e., at least two, copies of CTA1, multiple copies of LTA1, multiple copies of PTS1, at least one copy of CTA1 and at least one copy of LTA1, at least one copy of CTA1 and at least one copy of PTS1, at least one copy of LTA1 and at least one copy of PTS1 or at least one copy of CTA1, at least one copy of LTA1 and at least one copy of PTS1.

The second nucleotide sequence encodes a scFv that specifically binds to a surface marker on APCs. Accordingly, the fusion protein encoded by the nucleotide sequence and that comprises the scFv, in addition to the bacterial exotoxin, targets APCs specifically and thereby achieves a precise targeting and improved immunoenhancing effect.

In an embodiment, the second nucleotide sequence encodes a scFv that specifically binds to a surface marker on the dendritic cells (DCs). Hence, in this embodiment, the APCs that are targeted are DCs.

The particular APCs, preferably DCs, that are targeted is defined and based on which molecule or surface marker on the dendritic cells that the scFv specifically binds to. Hence, the surface marker is a protein anchored in or otherwise attached to the cell membrane of the APCs, preferably DCs, and is thereby accessible for the scFv to bind to an epitope in the protein. Such surface markers often comprise an extracellular part or domain, a transmembrane part or domain, and optionally an intracellular part or domain. For such a surface marker, the scFv preferably binds specifically to an epitope in the extracellular domain or part of the surface marker.

In an embodiment, the surface marker is a receptor, a membrane-bound protein or other molecule that is preferably expressed by the APCs, preferably DCs, and preferably accessible from the outside of the APCs, preferably DCs.

The specificity of a scFv can be determined based on affinity and/or avidity. The affinity, represented by the equilibrium constant for the dissociation of an antigen with the scFv (K_{D}), is a measure for the binding strength between an antigenic determinant and an antigen-binding site on the scFv. The lesser the value of K_{D}, the stronger the binding strength between the antigenic determinant and the scFv. Alternatively, the affinity can also be expressed as the affinity constant (K_{A}), which is 1/K_{D}. As will be clear to the skilled person, affinity can be determined in a manner known per se, depending on the specific antigen of interest.

Avidity is the measure of the strength of binding between a scFv and the pertinent antigen. Avidity is related to both the affinity between an antigenic determinant and its antigen binding site on the scFv and the number of pertinent binding sites present on the scFv.

Typically, scFv will bind to their antigen with a dissociation constant (K_{D}) of 10⁻⁵ to 10⁻¹² moles/liter (M) or less, and preferably 10⁻⁷ to 10⁻¹² M or less and more preferably 10⁻⁸ to 10⁻¹² M, i.e. with an association constant (K_{A}) of 10⁵ to 10¹² M⁻¹ or more, and preferably 10⁷ to 10¹² M⁻¹ or more and more preferably 10⁸ to 10¹² M⁻¹.

Generally, any K_{D} value greater than 10⁻⁴ M (or any K_{A} value lower than 104 M⁻¹) is generally considered to indicate non-specific binding.

Specific binding of a scFv to an epitope or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

Dendritic cells are traditionally divided into so-called conventional or classical dendritic cells (cDCs), previously called myeloid dendritic cells (mDCs), and plasmacytoid dendritic cells (pDCs), previously called interferon-producing cells. DCs can also be divided into blood and lymphoid DCs, skin tissue DCs or inflammatory/monocyte-derived DCs. cDCs arise from common monocyte-DC precursor cells in the bone marrow, while pDCs arise from common lymphoid progenitors.

cDCs are made up of at least two subsets: conventional or classical type 1 dendritic cells (cDC1s or mDC-1), which are major stimulators of CD4 and CD8 T cells leading to CD4 Th1 or regulatory T cell (Treg) and cytotoxic lymphocytes (CD8), respectively, and conventional or classical type 2 dendritic cells (cDC2s or mDC-2), which are important for CD4 T cell activation and, in particular, the development of follicular helper T cells (Tfh), TH17 and Th2 type of cells. cDCs secrete interleukin 12 (IL-12), IL-1 IL-6, IL-10, IL-23, IL-27, tumor necrosis factor (TNF) and chemokines and express toll like-receptors (TLR), such as TLR 2 and TLR 4. pDCs express the surface marker B220 and resemble plasma cells, and can express TLRs and produce high amounts of interferon-α (IFNα). cDCs, as opposed to pDCs, express the CD11c surface integrin alpha X (complement component 3 receptor 4 subunit) (ITGAX). Integrins are heterodimeric integral membrane proteins composed of an alpha chain and a beta chain. This protein combines with the beta 2 chain (ITGB2) to form a leukocyte-specific integrin referred to as inactivated-C3b (iC3b) receptor 4 (CR4). CD11c is a type I transmembrane protein found at high levels on human dendritic cells. CD11c⁺ dendritic cells are cDCs found in tissues and lymph nodes. Migrating CD11c⁺ dendritic cells express high levels of CD11c and MHC class II. Following activation in the tissues these cells migrate to the draining lymph nodes where they are central to priming of CD4 and CD8 T cells.

In an embodiment, the second nucleotide sequence encodes a scFv that specifically binds to a surface marker on conventional or classical dendritic cells (cDCs) of both the cDC1 and cDC2 types.

In a particular embodiment, the second nucleotide sequence encodes a scFv that specifically binds to thrombomodulin (TM), also referred to as cluster of differentiation 141 (CD141) or BDCA-3, which is a surface marker on cDC1s.

TM is an integral membrane protein expressed on the surface of endothelial cells and serves as a cofactor for thrombin. It reduces blood coagulation by converting thrombin to an anticoagulant enzyme from a procoagulant enzyme. Thrombomodulin is also expressed on human mesothelial cell, monocyte and cDCs.

In an embodiment, the second nucleotide sequence encodes a scFV that binds specifically to a surface marker on cDC1s. Hence, in this embodiment, the fusion protein targets cDC1s specifically.

In a particular embodiment, the second nucleotide sequence encodes a scFv that specifically binds to a surface marker selected from the group consisting of CD103, c-type lectin domain family 9 member A (Clec9A), XCR1, lymphocyte antigen 75 (LY75) (CD205), cell adhesion molecule 1 (CADM1), B- and T-lymphocyte attenuator (BTLA), dipeptidyl peptidase-4 (DPP4), and CD226, which are surface markers of cDC1s.

CD103, also referred to as integrin, alpha E (ITGAE), is an integrin protein that in human is encoded by the ITGAE gene. CD103 binds integrin beta 7 (β7 or ITGB7) to form the complete heterodimeric integrin molecule αEβ7. CD103⁺ is carried by cDC1 as well as by double positive cDC2 (CD103⁺CD11b⁺) DCs and these are key cells for priming of CD4 and CD8 T cells in the draining lymph node and for stimulating tissue resident memory T cells.

Clec9A is a group V C-type lectin-like receptor (CTLR) that functions as an activation receptor and is expressed on myeloid lineage cells, including cDC1s. CLEC9A⁺ cDC1s can cross-present and activate CD8 T-cell responses and CD4 Th1 or T reg type of responses.

XCR1 is also referred to as GPR5. XCR1⁺ cDC1 cells can cross-present and activate CD8 T cells and CD4 Th1 or Treg type of responses.

LY75, also referred to as CD205 or DEC205, is an endocytotic receptor on dendritic cells that recognizes dead cells in a pH-dependent fashion.

CADM1 is a cell adhesion molecule sometimes referred to as nectin-like protein 2 (NECL2). CADM1 is primarily expressed on pre-cDC1s.

BTLA, also referred to as CD272, is induced during activation of T cells and BTLA+ dendritic cells induce peripheral Treg cells.

DPP4, also referred to as adenosine deaminase complexing protein 2 or CD26, is associated with immune regulation, signal transduction, and apoptosis. DDP4⁺ DCs may be involved in inflammation.

CD226, also referred to as PTA1 platelet and T cell activation antigen 1 (PTA1) or DNAX Accessory Molecule-1 (DNAM-1), is expressed on cDCs.

In an embodiment, the second nucleotide sequence encodes a scFv that specifically binds to a surface maker selected from the group consisting of CD103, XCR1 and Clec9A.

Human cDC1 are defined as CD141⁺ (BDCA3⁺) dendritic cells and share many similarities with mouse cDC1, including expression of XCR1, CADM1, Clec9A, and CD103.

In a particular embodiment, the second nucleotide sequence encodes a scFv that specifically binds to CD103. In this embodiment, the subset of dendritic cells is CD103 expressing dendritic cells (CD103⁺ DCs). Such CD103⁺ DCs belong to the cDC1 subset or to the double positive cDC2 CD103⁺, CD11b⁺) subset.

In an embodiment, the second nucleotide sequence encoding a CD103-binding scFv comprises, preferably consists of, SEQ ID NO: 4.

CCCGGGGAGGTGCAGCTGGTGGAGTCTGGGGGAGGCTTAGTGCAGCCTGG
AAGGTCCCTGAAACTCTCCTGTGCAGCCTCAGGATTCACTTTCAGTGACT
ATTACATGGCCTGGGTCCGCCAGTCTCCAAAGAAGGGTCTGGAGTGGGTC
GCATCCATTAGTTATGAGGGTACTGACACTTACTATGGAGACTCCGTGAA
GGGCCGATTCACTATCTCCAGAGATAATGCAAAAAGCACCCTGTACCTGC
AAATGAACAGTCTGAGGTCTGAGGACACGGCCACTTATTATTGTGCAAGC
TTGAATGGCTATAACTATCGATACTTTGACTTCTGGGGCCCAGGAACCAT
GGTCACCGTGTCCTCAGGTGGAGGCGGTTCAGGCGGAGGTGGCTCTGGCG
GTGGAGGGAGCGGTGGAGGGAGTAACATCCAGATGACCCAGTCTCCTTCA
ATACTGTCTGCATCTGTGGGAGACAGAGTCACTCTCAGCTGCAAAGCAGG
TCAGAATATTAACAATTTCTTAGCCTGGTATCAGCAAAAGCTTGGAGAAG
CTCCCAAACTCCTGATATATAATGCAAACAGTTTGGAAACGGGCATCCCA
TCAAGGTTCAGTGGCAGTGGATCTGGCACAGATTACACACTCACCATCAG
CAGCCTGCGGCCTGAAGATGTTGCCACATATTTCTGCCAGCAGTATAACA
GTTGGTACACGTTTGGAGCTGGGACCAAGCTGGAACTGAAACGGGCTGAT
GCGGCCGCAAAGCTT

In another particular embodiment, the second nucleotide sequence encodes a scFv that specifically binds to LY75 (CD205). In this embodiment, the subset of dendritic cells is LY75⁺ expressing dendritic cells (LY75⁺ DCs or CD205⁺ DCs).

In an embodiment, the second nucleotide sequence encoding a CD205-binding scFv comprises, preferably consists of, SEQ ID NO 5:

CAGGTCAAGCTGCAGGAGTCAGGAGGAGGITTGGTACAGCCGGGGGGTTC
TCTGAGACTCTCCTGTGCAGCTTCTGGATTCACCTTCAATGATTTCTACA
TGAACTGGATCCGCCAGCCTCCAGGGCAGGCACCTGAGTGGTTGGGTGTT
ATTAGAAACAAAGGTAATGGTTACACAACAGAGGTCAATACATCTGTGAA
GGGGCGGTTCACCATCTCCAGAGATAATACCCAAAACATCCTCTATCTTC
AAATGAACAGCCTGAGAGCTGAGGACACCGCCATTTACTACTGTGCAAGA
GGCGGTCCTTATTACTACAGTGGTGACGACGCCCCTTACTGGGGCCAAGG
GACCACGGTCACCGTCTCGAGTGGTGGAGGCGGTTCAGGCGGAGGTGGCT
CTGGCGGTGGAGGGAGCGGTGGAGGGAGTGCAGACATTGTGCTCACTCAA
TCTCCAGCTTCCTTAGCTGTATCTCTGGGGCAGAGGGCCACCATCTCATG
CAGGGCCAGCAAAAGTGTCAGTACATCTGGCTATAGTTATATGCACTGGA
ACCAACAGAAACCAGGACAGCCACCCAGACTCCTCATCTATCTTGTATCC
AACCTAGAATCTGGGGTCCCTGCCAGGTTCAGTGGCAGTGGGTCTGGGAC
AGACTTCACCCTCAACATCCATCCTGTGGAGGAGGAGGATGCTGCAACCT
ATTACTGTCAGCACATTAGGGAGCTTTACACGTTCGGAGGGGGGACCAAG
CTGGAAATAAAACGGGCGGCCGCATAGA

cDC1s, identified by cell surface expression of XCR1, Clec9A, and/or CD103, are developmentally dependent on Interferon regulatory factor 8 (IRF8), also known as interferon consensus sequence-binding protein (ICSBP), and basic leucine zipper transcription factor, ATF-like 3 (BATF3). cDC1s cross-present antigens and prime cytotoxic CD8 T cell responses to intracellular pathogens and they are also linked to priming of CD4 T cells of the Th1 or Treg functions. cDC1s are a relatively homogeneous population.

In another embodiment, the second nucleotide sequence encodes a scFv that specifically binds to a surface marker on cDC2s.

cDC2s, are defined by the cell surface expression of CD11b, and comprise a heterogeneous population of cells exhibiting variable dependence on IRF4 and Notch signaling and have distinct functional roles in priming CD4 T cell responses of the Tfh, Th2 and Th17 type, in particular.

In an embodiment, the second nucleotide sequence encodes a scFv that specifically binds to a surface marker selected from the group consisting of CD1c/BDCA-1, CD11b, CD2, signal regulatory protein alpha (SIRPA)/CD172a, Fc epsilon RI (FCER1), leukocyte immunoglobulin-like receptor subfamily A member 2 (LILRA2), C-type lectin domain family 4 member A (Clec4A) and Clec10A, which are surface markers on cDC2s.

CD1c/BDCA-1 is member of a family of glycoproteins expressed on the surface of human antigen-presenting cells and are involved in presentation of lipid antigens to T cells. CD1c+ dendritic cells are present in human blood and tissues, and found to efficiently prime CD4 T cells.

CD11b is also referred to as Integrin alpha M (ITGAM). The CD11b⁺ DCs consist of a mixture of tissue cDCs and macrophages.

CD2 is also referred to as T-cell surface antigen T11/Leu-5, LFA-2, LFA-3 receptor, erythrocyte receptor or rosette receptor.

FCER1 is also referred to as high-affinity IgE receptor or FcεRI. FCER1 reinforces the ability of dendritic cells to internalize, process and present antigens by focusing these activity toward antigens bound to IgE.

LILRA1 is also referred to as ILT1 or CD85h, and Clec4A is also referred to as DCIR.

In an embodiment, the second nucleotide sequence encodes multiple copies of the scFv or encodes at least one copy of a first scFV and at least one copy of a second, different scFv.

The surface marker that the scFv specifically binds to is a surface maker of dendritic cells, and preferably to a subset of dendritic cells, such as cDCs, i.e., cDC1s and cDC2, cDC1s or cDC2s, more preferably cDC1s. The surface marker that is present on the dendritic cells may be present on other types of cells, or non-DCs, or even in other subtypes of dendritic cells. However, a surface marker for a given subset of dendritic cells is typically present in higher amounts and densities at the cell surface of the given subset of dendritic cells as compared to other subset of dendritic cells. For instance, CD103⁺ dendritic cells comprise significantly more CD103 molecules as compared to CD103⁻ dendritic cells.

In an embodiment, the nucleotide sequence comprising a third nucleotide sequence encoding at least one virus or bacterial epitope. Hence, in this embodiment, the nucleotide sequence comprises at least three subsequences. At least one virus epitope is preferably associated with a viral infection or disease, i.e., is expressed by a virus causing a viral infection or disease. This means that the at least one virus epitope will function as a vaccine antigen that can be processed and recognized by T cell receptors (TCRs) or the B cell receptors (BCRs). Correspondingly, the at least one bacterial epitope is preferably associated with a bacterial infection or disease, i.e., is expressed by bacterial causing a bacterial infection or disease. This means that the at least one bacterial epitope will function as a vaccine antigen that can be processed and recognized by TCRs or the BCRs.

The dendritic cells act as APCs and present the processed antigen in the form of peptides on major histocompatibility complex (MHC) molecules or complexes (MCS). The TCR recognizes the unique vaccine antigen peptide in a complex with MHC molecules and become activated, i.e., primed.

For TCR recognition, the antigen is processed into small fragments, i.e., peptides, inside the APCs and presented by a MHC class I or II molecule on the cell surface. These complexes are most often not sufficient to elicit an immune response and, therefore, there is need for an immunoenhancer, i.e., an adjuvant, which is latin for “to help”. The bacterial exotoxin encoded by the first nucleotide sequence acts as such an immunoenhancer.

In an embodiment, the third nucleotide sequence encodes at least one virus epitope and preferably at least one ectodomain of matrix protein 2 (M2e) epitope of influenza A virus. The extracellular domain of M2e protein is an evolutionarily conserved region in influenza A viruses and a promising epitope for designing a universal influenza vaccine. M2 is a tetrameric type III membrane protein containing 97 amino acids. The M2 protein can be divided into three parts: the extracellular N-terminal domain (M2e, positions 1-24), the transmembrane (TM) domain (positions 25-46) and the intracellular C-terminal domain (positions 47-97).

In an embodiment, the third nucleotide sequence encodes multiple M2e epitopes. For instance, the third nucleotide sequence could encode two, three, four or more M2e epitopes, preferably three M2e epitopes. In an embodiment, the multiple M2e epitopes encoded by the third nucleotide sequence may optionally be interconnected by a linker, e.g., M2e-linker-M2e-linker-M2e. In an embodiment, the optional linker is selected from the group consisting of at least one glycine (G), at least one serine (S), and a combination thereof, preferably GS.

In an embodiment, the M2e epitope comprises, preferably consists of, any of the following amino acid sequences SEQ ID NO: 6 (SLLTEVETPIRNEWGS), SEQ ID NO: 7 (SLLTEVETPIRNEWGCRCNDSSD) and SEQ ID NO: 8 (MSLLTEVETPTRNEWECRCSDSS).

In an embodiment, the third nucleotide sequence comprises, preferably consists of, SEQ ID NO: 9 (AGCCTGCTGACCGAGGTGGAGACCCCCATCAGGAACGAGTGGGGCAGC), SEQ ID NO: 10 (AGCCTGCTGACCGAGGTGGAGACCCCCATCAGGAACGAGTGGGGCTGCAGGTGCAACGACAGC AGCGAC), and SEQ ID NO: 11 (ATGAGCCTGCTGACCGAGGTGGAGACCCCCACCAGGAACGAGTGGGAGTGCAGGTGCAGCGAC AGCAGC).

The embodiments are, however, not limited to using M2e epitopes but could instead use other epitopes, such as protective epitopes from the nucleoprotein and other internal proteins (PB1, M1) from influenza A or B virus strains or relevant protective epitopes from other viruses, such as norovirus, rotavirus, coronavirus or HIV. The embodiments can also be used with bacterial epiotpes, such as an epitope from Chlamydia trachomatis.

For instance, the coronavirus conserved S surface glycoprotein and/or ORF1ab polyprotein (ORF1ab), or one or more parts thereof, could be used as epitope instead of, or together with, the M2e epitopes. In such a case, the epitope could be selected among one or more of the following amino acid sequences SEQ ID NO: 17 (FGAGAALQIPFAMQMAYRFNGI), SEQ ID NO: 18 (QLIRAAEIRASANLAATK) and SEQ ID NO: 19 ORF1ab (MMISAGFSL).

In a particular embodiment, the third nucleotide sequence encodes at least one severe acute respiratory syndrome coronovarius 2 (SARS-CoV-2) epitope and/or at least one SARS-CoV-1 epitope. For instance, the stem domain of the spike (S) glycol protein is an evolutionarily conserved region in all SARS-CoV viruses. S is a trimeric protein containing 1500 amino acids. In an embodiment, the third nucleotide sequence encodes at least one S epitopes, and preferably the third nucleotide sequence comprises one or multiple copies of the nucleotide sequence SEQ ID NO: 20 (TTTGGCGCGGGCGCGGCGCTGCAGATTCCGTTTGCGATGCAGATGGCGTATCGCTTTAAC), SEQ ID NO: 21 (CAGCTGATTCGCGCGGCGGAAATTCGCGCGAGCGCGAACCTGGCGGCGACCAAA), including one or multiple copies of a combination of SEQ ID NO: 20 and SEQ ID NO: 21 Alternatively, or in addition, the third nucleotide each sequence encodes at least one epitope of a coronavirus ORF1ab, such at SARS-CoV-2 ORF1ab. ORF1ab produces mainly polymerase enzyme of virus and consist 7000 amino acids. In such a case, the third nucleotide sequence comprises one or multiple copies of SEQ ID NO: 22 (ATGATGATTAGCGCGGGCTTTAGCCTG).

In some cases, the virus or bacterial epitope may be cleaved in the APCs, preferably DCs, and thereby will be less efficient as antigen. Any such problem could be solved by adding extra cleavage sits in the third nucleotide sequence. This then means that the fusion protein encoded by the nucleotide sequence will contain extra cleavage sites that can be targeted by proteases in the APCs, preferably DCs. Hence, protein cleavage is targeted against these dedicated extra cleavage sites, which in turn reduces the risk of undesired cleavage of the virus or bacterial epitopes. The particular type of and number of any such extra cleavage site can be selected based on information of the types of proteases present in the relevant APC, preferably DCs.

In an embodiment, the nucleotide sequences comprises, from a 5′ end to a 3′ end, the first nucleotide sequence, the third nucleotide sequence and the second nucleotide sequence. Hence, the fusion protein encoded by the nucleotide sequence preferably comprises, from the N-terminus to the C-terminus, the bacterial exotoxin (such as CTA1, LTA1 and/or PTS1), the at least one virus epitope (such as at least one M2e epitope) and the scFv.

In an embodiment, the nucleotide sequence comprises, preferably consist of, SEQ ID NO: 12 comprising, from a 5′ end to a 3′ end, the first nucleotide sequence encoding for CTA1, the third nucleotide sequence encoding for three copies of the M2e epitope and the second nucleotide sequence encoding for the CD103-binding scFv:

ATGAAATTTTTGGTGAACGTGGCCTTGGTGTTTATGGTGGTGTACATTTC
GTACATTTACGCCGACCCGTCGCCGGACGACAAATTGTACAGAGCCGACT
CGAGACCGCCGGACGAAATTAAACAATCGGGCGGCTTGATGCCGAGAGGC
CAATCGGAATACTTTGACAGAGGCACGCAAATGAACATTAACTTGTACGA
CCACGCCAGAGGCACGCAAACGGGCTTTGTGAGACACGACGACGGCTACG
TGTCGACGTCGATTTCGTTGAGATCGGCCCACTTGGTGGGCCAAACGATT
TTGTCGGGCCACTCGACGTACTACATTTACGTGATTGCCACGGCCCCGAA
CATGTTTAACGTGAACGACGTGTTGGGCGCCTACTCGCCGCACCCGGACG
AACAAGAAGTGTCGGCCTTGGGCGGCATTCCGTACTCGCAAATTTACGGC
TGGTACAGAGTGCACTTTGGCGTGTTGGACGAACAATTGCACAGAAACAG
AGGCTACAGAGACAGATACTACTCGAACTTGGACATTGCCCCGGCCGCCG
ACGGCTACGGCTTGGCCGGCTTTCCGCCGGAACACAGAGCCTGGAGAGAA
GAACCGTGGATTCACCACGCCCCGCCGGGCTGTGGCAACGCCCCGAGATC
GTCGGGGAATTCGTTGTTGACGGAAGTGGAAACGCCGATTAGAAACGAAT
GGGGCTCGAGATCGAACGACTCGTCGTTGTTGACGGAAGTGGAAACGCCG
ATTAGAAACGAATGGGGCTCGAGATCGAACGACTCGTCGTTGTTGACGGA
AGTGGAAACGCCGATTAGAAACGAATGGGGCTCGAGATCGAACGACTCGT
CGGACGGTTTAATTAAGTTTAAACCATATGAAGCCAGCTTCGAGGCCCAG
GGCGCCCTGGCCAACATCGCCGTGGACAAGGCCGCCAGCTTCGAGGCCCA
GGGCGCCCTGGCCAACATCGCCGTGGACAAGGCCGCCAGCTTCGAGGCCC
AGGGCGCCCTGGCCAACATCGCCGTGGACAAGGCCAGTTTAAACCCCGGG
AGCGGGGAGGTGCAGCTGGTGGAGTCTGGGGGAGGCTTAGTGCAGCCTGG
AAGGTCCCTGAAACTCTCCTGTGCAGCCTCAGGATTCACTTTCAGTGACT
ATTACATGGCCTGGGTCCGCCAGTCTCCAAAGAAGGGTCTGGAGTGGGTC
GCATCCATTAGTTATGAGGGTACTGACACTTACTATGGAGACTCCGTGAA
GGGCCGATTCACTATCTCCAGAGATAATGCAAAAAGCACCCTGTACCTGC
AAATGAACAGTCTGAGGTCTGAGGACACGGCCACTTATTATTGTGCAAGC
TTGAATGGCTATAACTATCGATACTTTGACTTCTGGGGCCCAGGAACCAT
GGTCACCGTGTCCTCAGGTGGAGGCGGTTCAGGCGGAGGTGGCTCTGGCG
GTGGAGGGAGCGGTGGAGGGAGTAACATCCAGATGACCCAGTCTCCTTCA
ATACTGTCTGCATCTGTGGGAGACAGAGTCACTCTCAGCTGCAAAGCAGG
TCAGAATATTAACAATTTTTTAGCCTGGTATCAGCAAAAGCTTGGAGAAG
CTCCCAAACTCCTGATATATAATGCAAACAGTTTGGAAACGGGCATCCCA
TCAAGGTTCAGTGGCAGTGGATTTGGCACAGATTACACACTCACCATCAG
CAGCCTGCGGCCTGAAGATGTTGCCACATATTTTTGCCAGCAGTATAACA
GTTGGTACACGTTTGGAGGTGGGACCAAGCTGGAAATGAAACGGGCTGAT
GCGGCCGCATATTCGAAGAGCTCCGACTACAAAGACGACGACGACAAACA
CCACCACCACCACCACTAG

The present invention also relates to nucleotide sequence as described above for use as a medicant. The invention further relates to a nucleotide sequence according to above for use as a vaccine adjuvant or, if the nucleotide sequence comprises the third nucleotide sequence encoding the at least one virus or bacterial epitope, for use as a vaccine.

The present invention also relates to an expression vector that comprises the nucleotide sequence according to the invention.

The expression vector comprising the nucleotide sequence can be expressed, i.e., transcribed and translated, in a host cell comprising the expression vector. In an embodiment, the expression vector is selected among plasmids, episomal plasmids and virus vectors.

In an embodiment, the expression vector is a virus vector. In a particular embodiment, the virus vector is selected from a group consisting of a lentiviral vector, an adenoviral vector, an adeno-associated viral vector, a retroviral vector and a hybrid vector.

Lentiviruses are a subclass of retroviruses. They are adapted as gene delivery vehicles (vectors) thanks to their ability to integrate into the genome of non-dividing cells, which is the unique feature of lentiviruses as other retroviruses can infect only dividing cells. The viral genome in the form of RNA is reverse-transcribed when the virus enters a cell to produce DNA, which is then inserted into the genome at a position by the viral integrase enzyme. The vector, now called a provirus, remains in the genome and is passed on to the progeny of the cell if it divides. For safety reasons lentiviral vectors typically never carry the genes required for their replication. To produce a lentivirus, several plasmids are transfected into a so-called packaging cell line, commonly HEK 293. One or more plasmids, generally referred to as packaging plasmids, encode the virion proteins, such as the capsid and the reverse transcriptase. Another plasmid contains the genetic material to be delivered by the vector. It is transcribed to produce the single-stranded RNA viral genome and is marked by the presence of the ψ (psi) sequence. This sequence is used to package the genome into the virion.

Retroviruses are one of the mainstays of current gene therapy approaches. The recombinant retroviruses, such as the Moloney murine leukemia virus, have the ability to integrate into the host genome in a stable fashion. They contain a reverse transcriptase that allows integration into the host genome. Retroviral vectors can either be replication-competent or replication-defective. Replication-defective vectors are the most common choice because the viruses have had the coding regions for the genes necessary for additional rounds of virion replication and packaging replaced with other genes, or deleted. These virus are capable of infecting various cells and delivering their viral payload, but then fail to continue the typical lytic pathway that leads to cell lysis and death.

If the expression vector is a lentiviral or retroviral vector then the nucleotide sequence encoding the fusion protein is preferably a RNA sequence.

Adenoviral vector as used herein include adenovirus vectors and adenovirus-derived virus vectors. Adenoviral DNA does not integrate into the genome and is not replicated during cell division. An adeno-derived virus vector is based on an adenovirus but in which various modifications have been done, such as relating to the nucleotide sequences coding replication proteins, regulation protein, viral surface proteins, etc. Adeno-associated virus (AAV) is a small virus that infects humans and some other primate species. AAV can infect both dividing and non-dividing cells and may incorporate its genome into that of the host cell. Moreover, AAV mostly stays as episomal, performing long and stable expression. Whereas AAV packages a single strand of DNA and requires the process of second-strand synthesis, self-complementary adeno-associated virus (scAAV) packages both strands which anneal together to form double stranded DNA. By skipping second strand synthesis, scAAV allows for rapid expression in the cell. If the expression vector is an adenoviral vector then the nucleotide sequence encoding the fusion protein is preferably a DNA sequence.

A hybrid vector is a vector virus that is genetically engineered to have qualities of more than one vector. For instance, a hybrid vector may be a combination of an adenovirus and a lentivirus.

The expression vector may also comprise a promoter controlling expression of the nucleotide sequence. Hence, in such an embodiment, the nucleotide sequence is operably controlled by the promoter to direct expression of the nucleotide sequence in a cell, such as a host cell. The promoter may be an endogenous promoter of the expression vector, such as a viral promoter in the case of a virus vector. In an alternative embodiment, other promoters may be used, including exogenous promoters, inducible promoters or constitutive promoters that are active or inducibly active in a subject.

The invention further relates to a cell comprising a nucleic sequence according to the invention and/or an expression vector according to the invention.

The cell could be a bacterial cell, a fungal cell, a yeast cell, an animal cell, such as a mammalian cell, e.g., a human cell. The cell may, for instance, be of a cell or a cell line of human origin that can be propagated in vitro.

The cell can advantageously be cultured in vitro for production and, preferably, release of the fusion protein into the culture medium. For instance, the cell could be a bacterial cell, a fungal cell or a yeast cell selected for culturing and fusion protein production in a bioreactor, a fermenter or other, preferably large scale, protein production facility.

Another aspect of the invention relates to a fusion protein comprising a bacterial exotoxin and a single chain antibody fragment (scFv) that specifically binds to a surface marker on antigen presenting cells.

In an embodiment, the bacterial exotoxin is selected from a group consisting of an A1 subunit of a bacterial enterotoxin and a pertussis toxin.

In a particular embodiment, the bacterial eneterotoxin is selected from the group consisting of cholera toxin (CT) and Escherichia coli heat labile enterotoxin (LT).

In an embodiment, the A1 subunit of the bacterial enterotoxin is the A1 subunit of cholera toxin (CTA1). In a particular embodiment, the CTA1 comprises, preferably consists of, the amino acid sequence of SEQ ID NO: 13:

MDDKLYRADSRPPDEIKQSGGLMPRGQSEYFDRGTQMNINLYDHARGTQT
GFVRHDDGYVSTSISLRSAHLVGQTILSGHSTYYIYVIATAPNMFNVNDV
LGAYSPHPDEQEVSALGGIPYSQIYGWYRVHFGVLDEQLHRNRGYRDRYY
SNLDIAPAADGYGLAGFPPEHRAWREEPWIHHAPPGCGNAPRSSGST

In another embodiment, the A1 subunit of the bacterial enterotoxin is the A1 subunit of E. coli heat labile enterotoxin (LTA1). In a particular embodiment, the LTA1 comprises, preferably consists of, the amino acid sequence of SEQ ID NO: 14:

MKNITFIFFILLASPLYANGDKLYRADSRPPDEIKHSGGLMPRGHNEYFD
RGTQININLYDHARGTQTGFVRYDDGYVSTSLSLRSAHLAGQSILSGYST
YYIYVIATAPNMFNVNDVLGVYSPHPYEQEVSALGGIPYSQIYGWYRVNF
GVIDERLHRNREYRDRYYRNLNIAPAEDGYRLAGFPPDHQAWREEPWIHH
APQGCGNSSRTI

In an embodiment, the pertussis toxin is pertussis toxin subunit S1 (PTS1). In a particular embodiment, the PTS1 comprises, preferably consists of, the amino acid sequence of SEQ ID NO: 15:

DDPPATVYRYDSRPPEDVFQNGFTAWGNNDNVLDHLTGRSCQVGSSNSAF
VSTSSSRRYTEVYLEHRMQEAVEAERAGRGTGHFIGYIYEVRADNNFYGA
ASSYFEYVDTYGDNAGRILAGALATYQSEYLAHRRIPPENIRRVTRVYHN
GITGETTTTEYSNARYVSQQTRANPNPYTSRRSVASIVGTLVRMAPVIGA
CMARQAESSEAMAAWSERAGEAMVLVYYESIAYSF

In an embodiment, the fusion protein lacks any subunit of the bacterial enterotoxin besides the A1 subunit, if the bacterial exotoxin is a bacterial enterotoxin, and lacks any subunit of the pertussis toxin besides the S1 subunit, if the bacterial exotoxin is a pertussis toxin.

The fusion protein may comprise multiple copies of the CTA1, multiple copies of the LTA1, multiple copies of the PTS1, at least one copy of the CTA1 and at least one copy of the LTA1, at least one copy of the CTA1 and at least one copy of the PTS1, at least one copy of the LTA1 and at least one copy of the PTS1, or at least one copy of the CTA1, at least one copy of the LTA1 and at least one copy of the PTS1.

In an embodiment, the scFv specifically binds to a surface marker on dendritic cells (cDCs).

In a particular embodiment, the scFv specifically binds to a surface marker on conventional or classical dendritic cells (cDCs). In a particular embodiment, the scFv specifically binds to thrombomodulin (TM), which is a surface marker on cDCs. Hence, in such an embodiment, the scFv binds specifically to the cDC subtype of dendritic cells.

In another embodiment, the scFv specifically binds to a surface marker on conventional or classical type 1 dendritic cells (cDC1s). In a particular embodiment, the scFv specifically binds to a surface maker selected from the group consisting of cluster of differentiation 103 (CD103), c-type lectin domain family 9 member A (Clec9A), XCR1, lymphocyte antigen 75 (LY75)/CD250, cell adhesion molecule 1 (CADM1), B- and T-lymphocyte attenuator (BTLA), dipeptidyl peptidase-4 (DPP4), and CD226, preferably the second nucleotide sequence encodes a scFv that specifically binds to a surface maker selected from the group consisting of CD103, XCR1 and Clec9A. These proteins are surface markers on cDC1s. Hence, in this embodiment, the scFv binds specifically to the cDC1 subtype of dendritic cells.

In a further embodiment, the second nucleotide sequence encodes a scFv that specifically binds to a surface marker on conventional or classical type 2 dendritic cells (cDC2s). In a particular embodiment, the scFv specifically binds to a surface maker selected from the group consisting of cluster of differentiation 1c (CD1c/BDCA-1), CD11b, CD2, Fc epsilon RI (FCER1), signal regulatory protein alpha (SIRPA)/CD172a, leukocyte immunoglobulin-like receptor subfamily A member 2 (LILRA2), C-type lectin domain family 4 member A (Clec4A) and Clec10A. These proteins are surface markers of cDC2s. Hence, in this embodiment, the scFv binds specifically to the cDC2 subtype of dendritic cells.

In a particular embodiment, the scFv specifically binds to CD103 and the subset of dendritic cells is CD103 expressing dendritic cells (CD103⁺ dendritic cells), which include the cDC1 subtype and the double positive cDC2 (CD103⁺, CD11b⁺) subtype. In another particular embodiment, the scFv specifically binds to LY75 (CD205) and the subset of dendritic cells is LY75 (CD205) expressing dendritic cells (LY75⁺ or CD205⁺ dendritic cells). In a further particular embodiment, the scFv specifically binds to XCR1 and the subset of dendritic cells is XCR1 expressing dendritic cells (XCR1⁺ dendritic cells). In yet another particular embodiment, the scFv specifically binds to Clec9A and the subset of dendritic cells is Clec9A expressing dendritic cells (Clec9A⁺ dendritic cells). In a particular embodiment, the scFv specifically binds to CD11c and the subset of dendritic cells is CD11c expressing dendritic cells (CD11c⁺ dendritic cells), cDC1 and cDC2 cells.

In an embodiment, the scFV comprises, preferably consists of, the amino acid sequence of SEQ ID NO: 16:

GDSVKGRFTISRDNAKSTLYLQMNSLRSEDTATYYCASLNGYNYRYFDFW
GPGTMVTVSSGGGGSGGGGSGGGGSGGGSNIQMTQSPSILSASVGDRVTL
SCKAGQNINNFLAWYQQKLGEAPKLLIYNANSLETGIPSRFSGSGSGTDY
TLTISSLRPEDVATYFCQQYNSWYTFGAGTKLELKRADAAA

scFv can be produced according to various techniques well know in the art including, but not limited, to phage display technology. Once cloned, it is possible to increase the affinity and specificity of antigen binding by mimicking somatic hypermutation during an immune response. Phage display technology makes it even be possible to replace the existing practices of animal immunization and hybridoma development through a bacterial system capable of synthesizing and expressing practically unlimited quantities of antibodies to almost any antigen.

In order to get scFv antibody fragments, mRNA is first isolated from hybridoma (or spleen, lymph cells, and bone morrow) followed by reverse transcription into cDNA to serve as a template for antibody genes amplification using PCR. With this technique, large libraries with a diverse range of antibody VH and VL genes can be created. A biopanning step is used to get the scFv antibody fragment with the best affinity and specificity. In the construction of scFv, the order of the domains can be either VH-linker-VL or VL-linker-VH.

scFv antibody fragment expression and production is a routine work to support antibody discovery with an antibody library. When the antibody genes are successfully cloned and sequenced, scFv fragments can be readily expressed in microbial expression system such as E. coli. It can also be expressed in mammalian systems (e.g., HEK293 cells) through transient transfection.

A suitable purification tag is typically added to the C-terminus of the antibody scFv fragment protein. Commonly used purification tags are poly-histidine tag, FLAG-tag, HA-tag, and Myc-tag. A protease cleavage site can be included between the tag and the scFv to allow tag-removal after purification.

scFv that can be used according to the invention are disclosed in, for instance, Molecular Immunology (2005) 42(8): 979-985; PLoS One (2012) 7(9): e45102; Clinical Cancer Research (2009), 15: 4612-4621; Blood (2010) 116(13): 2277-2285; and U.S. Patent Application Nos. 2004/0146948 and 2020/0347138.

In an embodiment, the fusion protein comprises multiple scFvs, such as at least one first scFv and at least one second, different scFv or multiple copies of a same scFv.

In an embodiment, the fusion protein comprises at least one virus or bacterial epitope. Various such virus epitopes can be used according to the embodiments as previously discussed herein, for instance, epitopes or antigens from influenza A or B virus, norovirus, rotavirus, coronavirus or HIV. An example of bacterial epitopes is epitopes of Chlamydia trachomatis.

In an embodiment, the at least one virus epitope is at least one ectodomain of matrix protein 2 (M2e) epitope of influenza A virus.

In a particular embodiment, the M2e epitope comprises, preferably consists of, any of the following amino acid sequences SEQ ID NO: 6 (SLLTEVETPIRNEWGS), SEQ ID NO: 7 (SLLTEVETPIRNEWGCRCNDSSD) and SEQ ID NO: 8 (MSLLTEVETPTRNEWECRCSDSS).

In another particular embodiment, the at least one virus epitope is at least one coronavirus epitope, preferably selected from the group consisting of SEQ ID NO: 17 (FGAGAALQIPFAMQMAYRFNGI), SEQ ID NO: 18 (QLIRAAEIRASANLAATK) and SEQ ID NO: 19 (MMISAGFSL).

The fusion protein could comprise a single virus or bacterial epitope, multiple copies of the same virus or bacterial epitope or indeed different virus epitopes from the same or different viruses or bacterial. For instance, the fusion protein may comprise multiple M2e epitopes, preferably three M2e epitopes. In such a case, the multiple virus epitopes, such as M2e epitopes, are optionally interconnected by a linker selected from the group consisting of at least one glycine (G), at least one serine (S), and a combination thereof, preferably GS. The fusion protein may comprise multiple coronavirus epitopes or at least one coronavirus epitope, such as at least one epitope from S glycoprotein and/or ORF1ab, and at least one M2e epitope.

Generally, the fusion protein, or rather the virus or bacterial epitope(s) therein, is more immunogenic if the fusion protein comprises multiple copies of the virus or bacterial epitope as compared to merely containing a single copy of the virus or bacterial epitope. Hence, in an embodiment, the fusion protein comprises N copies of the virus or bacterial epitope, preferably as N tandem repeats for the virus or bacterial epitope and where N is equal to or larger than 2, preferably selected within a range of 2 to 10, preferably within a range of 2 to 8, more preferably within a range of 2 to 6, such as within a range of 2 to 4, for instance N=3. Hence, the fusion protein may contain 2, 3, 4, 5, 6, 7, 8, 9 or 10 copies of the virus or bacterial epitopes.

In an embodiment, the fusion protein comprises, from an N-terminus to a C-terminus, the A1 subunit of the bacterial enterotoxin, the at least one virus or bacterial epitope and the scFv. The different subunits of the fusion proteins may optionally be interconnected by a linker selected from the group consisting of at least one glycine (G), at least one serine (S), and a combination thereof, preferably GS.

In an embodiment, the fusion protein is water-soluble. CTA1 and LTA1 alone generally have low solubility in physiological aqueous solutions. However, the fusion protein of the embodiments that, in addition to CTA1 and/or LTA1, also comprises at least scFv and optionally at least one virus or bacterial epitope has higher solubility as compared to CTA1 and LTA1 alone. Accordingly, the fusion protein is preferably water soluble.

In an embodiment, the fusion protein specifically binds to the subset of dendritic cells. Thus, the presence of the scFv in the fusion protein means that the fusion protein can specifically target and bind to a particular subset of dendritic cells that express a molecule, such as membrane bound protein or transmembrane protein, which the scFv has specificity for and specifically binds to. This targeting effect of the fusion protein due to the presence of the scFv increases the efficacy of the fusion protein as an adjuvant or, if it comprises at least one virus epitope, as a vaccine. For instance, a scFv that specifically binds to CD103 would specifically target a CD103⁺ subset of mucosal dendritic cells. This subset of dendritic cells is important for stimulation of effector and central as well as tissue resident memory CD4 and CD8 T cells. These populations of T cells assists other lymphocytes, including maturation of B cells into plasma cells and memory B cells, and activation of cytotoxic CD8 T cells and macrophages and many other cells belonging to the innate immune system. Once the CD4 T cells have been activated by the CD103⁺ dendritic cells, they divide rapidly and develop into functional subsets that secrete specific cytokines that regulate and assist the immune response.

This subset of dendritic cells is important for stimulation of effector and central as well as tissue resident memory CD4 and CD8 T cells. These populations of T cells assists other lymphocytes, including maturation of B cells into plasma cells and memory B cells, and activation of cytotoxic CD8 T cells and macrophages and many other cells belonging to the innate immune system. Once the CD4 T cells have been activated by the CD103⁺ dendritic cells, they divide rapidly and develop into functional subsets that secrete specific cytokines that regulate and assist the immune response.

Hence, the fusion protein embodiments comprising at least M2e epitope are capable of inducing Th17 cells when administered to a subject, preferably a human subject.

In an embodiment, the fusion protein specifically binds to the dendritic cells through the binding of the scFv part of the fustin protein to the surface marker on the dendritic cells. The fusion protein is preferably internalized by the dendritic cells. Thus, the fusion protein of the embodiments can preferably be taken up and internalized by the targeted dendritic cells. The dendritic cells may then process the fusion protein and, if the fusion protein comprises at least one virus epitope, process and present the at least one virus or bacterial epitope together with MHC molecules on the cell surface.

The fusion protein of the embodiments can be used as an adjuvant for vaccines, and in particular for mucosal vaccines. An aspect of the invention therefore relates to an adjuvant composition comprising a fusion protein according to the invention and a pharmaceutically acceptable carrier.

The fustion protein of the embodiments can be used as an adjuvant for various vaccines and in particular mucosal vaccines. The fusion protein may then be used in combination with an agent that provides active acquired immunity to a particular disease. This agent may, for instance, be a weakend or killed form of a disease-causing microorganism, its toxin(s) and/or a surfacer internal protein of the disease-causing microorganism.

In such a case, the agent may be admixed with the fusion protein and the pharmaceutical acceptable carrier to achieve a vaccine composition. Hence, in such an embodiment, the agent and the fusion protein are administered together to a subject. Alternatively, the agent and the fusion protein or the adjuvant composition may be administered separately to the subject in any order, and using the same or different administration routes.

The adjuvant effect of the fustion protein on the immunogenicity of the vaccine antigen is, however, improved when the immune protective virus or bacterial epitope(s) is(are) included in the fusion protein.

In such a case, the at least one virus or bacterial epitope will be much more immunogenic and protective as compared to having separated fusion protein (adjuvant) and virus or bacterial epitopes (agent).

Hence, another aspect of the invention relates to a vaccine composition comprising a fusion protein according to the invention and comprising at least one virus or bacterial epitope and a pharmaceutically acceptable carrier.

The pharmaceutical acceptable carrier of the adjuvant or vaccine composition can be selected based on the particular administration route. In an embodiment, the pharmaceutical acceptable carrier is water or an aqueous solution, such as saline, or a buffered aqueous solution, such as phosphate-buffered saline (PBS). In an embodiment, the pharmaceutical acceptable carrier is selected from the group consisting of liposomes, polymeric micelles, microspheres, nanoparticles and nanofibers.

A potential problem with vaccines, and in particular mucosal vaccines, is that mucosal administration requires protection of the antigen from enzymatic degradation and rapid clearance, which will lower immunogenicity of the vaccine at mucosal surfaces. Therefore, the pharmaceutical acceptable carrier could be used to increase vaccine immunogenicity and resistance to degradation. For instance, lipid-based nanoparticles (LNP) represent a promising and versatile option, especially since their physicochemical properties are easily and vastly customizable. These nanoparticles can function by enhancing delivery of the vaccine, but they can also increase immunogenicity by adding adjuvant properties to the vaccine, a phenomenon that is supported by studies showing that when vaccine and LNP administrations were separated by up to 48 h, an enhanced vaccine immunogenicity was still recorded.

Hence, in an embodiment, the adjuvant or vaccine composition comprises lipid nanoparticles (LNPs).

LNPs are effective as vaccine adjuvants due to their relative simplicity, safety and flexibility in allowing loading with protein or plasmids. By varying the lipid composition, more complex particle morphologies, e.g., cubic, hexagonal, or sponge phase structures can be created, including nanoscale disc-shaped more rigid lipid-protein complexes. LNPs are also known to protect adjuvants and vaccine antigens against degradation and prevent loss of enzymatic activity. However, while LNPs can prime immune responses in vivo, they show no selectivity for distinct DC subsets, and are also taken up by non-DCs, such as B cells and macrophages. Therefore, targeting of LNPs to distinct subsets of DCs can be achieved by the fusion protein of the present invention, which may also function as a vaccine epitope carrier and a strong immunoenhancer.

The fusion protein of the invention may be loaded into the pharmaceutical acceptable carrier, adsorbed or absorbed onto a surface of the pharmaceutical acceptable carrier and/or covalently coupled to the pharmaceutical acceptable carrier.

In embodiment, the fusion protein is covalently coupled to a LNP, optionally using a spacer or linker. For instance, the fusion protein may be covalently coupled to the LNP by means of linker selected from the group consisting of a polymer linker, poly(ethylene glycol) (PEG), a glycan, a polypeptide and an oligonucleotide.

In a particular embodiment, the linker is a PEG linker or spacer, such as a PEG(2000)-spacer.

In an embodiment, the fusion protein is covalently bound to LNPs using a thiol-maleimide reaction after converting primary amines to thiol groups using Traut's reagent (2-iminothiolane hydrochloride). Briefly, Traut's reagent (0.02 mg/ml in HBS: 10 mM HEPES, 150 nM NaCl, with 2 mM EDTA, pH=7.8) and fusion protein (1.6 mg/ml in 10 mM NaH₂PO₄, 0.16 M NaCl, pH 7.4) are mixed to a volume ratio of 5:3 and incubated for 20 minutes at 4° C. Particles at a lipid concentration of 4 mM are added to freshly thiolated fusion protein at a volume ratio of 5:4 and incubated at room temperature for 1 h with gentle shaking. Unreacted fusion protein may be removed using Amicon Ultra 100 kDa cut-off centrifugal filters (VWR, Sweden) as follows: 125 μl NaAc saline and approximately 290 μl LNP suspension are added to each filter and centrifuged (5 min or until approximately 250 μl solution remain, 8000×g, 10° C.) followed by a further dilution with 150 μl NaAc saline in each filter and another centrifugation (5 min or until approximately 250 μl solution remain, 8000×g, 10° C.). The suspension may be recovered by inverting the filters and centrifuging (1 min, 8000×g, 10° C.).

In an embodiment, the LNP comprises, on average, at least 50 covalently coupled fusion proteins, preferably at least 75 covalently coupled fusion proteins, and more preferably at least 100 covalently coupled fusion proteins.

In an embodiment, the LNP has an average diameter within a range of from 50 nm to 250 nm, preferably within a range of from 75 nm to 200 nm, and more preferably within a range of from 100 to 150 nm.

In an embodiment, the LNPs are in a gel phase. As compared to their fluid phase counterparts, gel phase LNPs are more efficient at improving antigen presentation. They are also superior at upregulating the co-stimulatory molecules CD80 and CD86, as well as increasing the release of the cytokines IL-6 and IL-1β. In a particular embodiment, the LNPs are liposomes comprising 1,2-distearoyl-sn-glycero-3-phosphocholine (DSPC). Such DSPC liposomes form LNPs that are in a gel phase.

Other examples of LNPs that can be used according to the embodiments include 1,2-dioleoyl-sn-glycero-3-phosphocholine (DOPC). These DOPC LNPs are in fluid phase.

Further aspects of the embodiments include a fusion protein according to the invention for use as a medicament, for use as an adjuvant and/or for use as a vaccine. In the latter use, i.e., as a vaccine, the fusion protein comprises at least one vaccine epitope. Correspondingly, the invention relates to the use of a fusion protein according to the invention for the manufacture of a medicament, for the manufacture of an adjuvant or for the manufacture of a vaccine.

The embodiments of the present invention can be used as a vaccine against various diseases including, but not limited to, infectious disease including, but not limited to, infectious diseases caused by viruses, i.e., viral diseases, or by bacteria, i.e., bacterial diseases, and cancer diseases. Particularly interesting are infections that are caused by viruses or bacteria that gain access to the body through mucosal membranes. This category includes that infect the respiratory, gastrointestinal and genital tracts. Illustrative examples include diseases caused by Chlamydia trachomatis, influenza A or B virus, norovirus, rotavirus, coronavirus or HIV

The invention also relates to a liposome or LNP made of 1,2-distearoyl-sn-glycero-3-phosphocholine (DSPC) for use as an adjuvant. In such case, the liposome or LNP is in a gel state.

In an embodiment, the LNP has an average diameter within a range of from 50 nm to 250 nm, preferably within a range of from 75 nm to 200 nm, and more preferably within a range of from 100 to 150 nm.

In an embodiment, the liposome or LNP is in a gel state or gel-phase state at a physiological temperature, preferably at 37° C.

In an embodiment, the liposome or LNP comprises at least one protein covalently coupled to the liposome or LNP, optionally using a spacer or linker. In an embodiment, the linker or spacer is selected from the group consisting of a polymer linker, poly(ethylene glycol) (PEG), a glycan, a polypeptide and an oligonucleotide. In a particular embodiment, the linker or spacer is a PEG linker or spacer, such as a PEG(2000)-spacer.

In an embodiment, the at least one protein is covalently bound to lipsome or LNP using a thiol-maleimide reaction after converting primary amines to thiol groups using Traut's reagent (2-iminothiolane hydrochloride). Briefly, Traut's reagent (0.02 mg/ml in HBS: 10 mM HEPES, 150 nM NaCl, with 2 mM EDTA, pH=7.8) and protein (1.6 mg/ml in 10 mM NaH₂PO₄, 0.16 M NaCl, pH 7.4) are mixed to a volume ratio of 5:3 and incubated for 20 minutes at 4° C. Lipsomes or LNPs at a lipid concentration of 4 mM are added to freshly thiolated protein at a volume ratio of 5:4 and incubated at room temperature for 1 h with gentle shaking. Unreacted protein may be removed using Amicon Ultra 100 kDa cut-off centrifugal filters (VWR, Sweden) as follows: 125 μl NaAc saline and approximately 290 μl LNP suspension are added to each filter and centrifuged (5 min or until approximately 250 μl solution remain, 8000×g, 10° C.) followed by a further dilution with 150 μl NaAc saline in each filter and another centrifugation (5 min or until approximately 250 μl solution remain, 8000×g, 10° C.). The suspension may be recovered by inverting the filters and centrifuging (1 min, 8000×g, 10° C.).

In an embodiment, a single type of protein is covalently bound to the liposome or LNP. In another embodiment, a mixture of multiple different proteins is covalently bound to the liposome or LNP.

The at least one protein covalently attached to the lipsome or LNP can be any protein or peptide that can be covalently coupled to the liposome or LNP, preferably as described above. Illustrative, but non-limiting, examples of such proteins include protein drugs, antibodies, enzymes, fusion proteins, acellular protein vaccines, protein labels, etc.

The present invention also relates to a method of vaccinating a subject against a bacterial or virus infection or disease. The method comprises administering a vaccine composition according to the invention to the subject.

In an embodiment, administering the vaccine composition comprises intranasal, oral, rectal, intravaginal or intrapulmonary administration of the vaccine composition. In a particular embodiment, the vaccine composition is administered as an aerosol or spray.

The subject is preferably a human subject. The present invention can, however, also be used for veterinary purposes, in which the subject is an animal, preferably a mammal, such as cat, dog, horse, cow, sheep, goat, rabbit, guinea pig, rat or mouse.

The present invention also encompasses nucleotide sequences and amino acid sequences that are homologues to the nucleotide sequences and amino acid sequences presented herein. A “homologue” of a nucleotide sequence or amino acid sequence has a substantial sequence identity, i.e., at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100% to the nucleotide sequence or amino acid sequence.

In particular, the present invention encompasses nucleotide sequence that may be codon adapted or optimized based on the particular host cell or species. Hence, the invention also encompass codon adapted or optimized versions of the nucleotide sequences as disclosed herein. Such codon adapted nucleotide sequence preferably encode the same amino acid sequence as the original nucleotide sequence but with at least one codon different form the corresponding codon in the original nucleotide sequence. Generally, the amino acid sequence of most proteins can be encoded by a number of different nucleotide sequences due to the degenerate nature of the genetic code. Nucleotide sequences encoding the same amino acid sequence via different codon assignments can vary dramatically in the amount of protein expressed in different host cells.

As used herein “sequence identity” refers to the extent to which two optimally aligned nucleotide acid or amino acid sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear nucleic acid sequence of a reference (“query”) nucleic acid molecule (or its complementary strand) as compared to a test (“subject”) nucleic acid molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or amino acid sequences, refers to two or more sequences or subsequences that have at least about 70%, least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 residues to about 150 residues in length. Thus, in some embodiments of the invention, the substantial identity exists over a region of the sequences that is at least about 16, at least about 18, at least about 22, at least about 25, at least about 30, at least about 40, at least about 50, about 60, about 70, about 80, about 90, about 100, about 110, about 120, about 130, about 140, about 150, or more residues in length, and any range therein.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can also be considered to be substantially identical when the two sequences hybridize to each other under stringent conditions. In some representative embodiments, two nucleotide sequences considered to be substantially identical hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1×SSC, 0.1% SDS at 50° C. or in 7% sodium dodecyl sulfate (SDS), 0.5 M NaPO₄, 1 mM EDTA at 50° C. with washing in 0.5×SSC, 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1×SSC, 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

In particular embodiments, a further indication that two nucleotide sequences or two amino acid sequences are substantially identical can be that the peptide or encoded by the first nucleic acid is immunologically cross reactive with, or specifically binds to, the peptide protein encoded by the second nucleic acid. Thus, in some embodiments, a polypeptide can be substantially identical to a second polypeptide, for example, where the two polypeptides differ only by conservative substitutions.

Examples of such conservative substitutions include substitution among aliphatic amino acids, i.e., glycine, alanine, valine, leucine and isoleucine, substitution among hydroxyl or sulfur-containing amino acids, i.e., serine, cysteine, threonine and methionine, substitution among aromatic amino acids, i.e., phenylalanine, tyrosine and tryptophan, substitution among basic amino acids, i.e., histidine, lysine and arginine, and substitution among acidic amino acids and their amides, i.e., aspartate, glutamate, asparagine and glutamine.

### EXAMPLES

### Example 1

DNA construction, expression and purification of different epitope bearing CTA1-CD103 fusion molecules were produced both prokaryoutic Escherichia coli and eukaryotic insect cell systems.

### DNA Construction

Total cellular RNA from M290 Hybridoma was extracted using RNA easy mini kit QIAGEN). Complementary DNA (cDNA) synthesis on extracted messenger RNA (mRNA) was carried out using Phusion reverse transcription polymerase chain reaction (RT-PCR) Kit (Finnzymes F-546S); Template RNA 5 μl (1 μg), 10 mM dNTP mix 1 μl, Oligo(dT) primer 1 μl, RNase-free H₂O added to 10 μl. Primer extension was performed at 25° C. 10 min, cDNA synthesis at 40° C. 30 min, and reaction termination at 85° C. 5 min.

The amplified products were sequenced.

>4-VL_HBS-rck (34 . . . 501 of sequence)
(SEQ ID NO: 23)
GATAGATACAGTTGGTGCAGCATCAGCCCGTTTCATTTCCAGCTTGGTCC
CACCTCCAAACGTGTACCAACTGTTATACTGCTGGCAAAAATATGTGGCA
ACATCTTCAGGCCGCAGGCTGCTGATGGTGAGTGTGTAATCTGTGCCAAA
TCCACTGCCACTGAACCTTGATGGGATGCCCGTTTCCAAACTGTTTGCAT
TATATATCAGGAGTTTGGGAGCTTCTCCAAGCTTTTGCTGATACCAGGCT
AAAAAATTGTTAATATTCTGACCTGCTTTGCAGCTGAGAGTGACTCTGTC
TCCCACAGATGCAGACAGTATTGAAGGAGACTGGGTCATCTGGATGTTAC
ATCTCAGGGCTGGGAGCCAGAGCAACACCAGCCCTAAGAGTTGAACTGGA
GCCATCATGACTGGCCTGTGTCCTGTCTGAGACTGAATACCAAAGCCTGC
CGGGGGGGGGGGGGAACA
>8-VH_HBS-rG2a (7 . . . 570 of sequence)
(SEQ ID NO: 24)
TAAGTACTTTTGAGAGCAGTTCCAGGAGCCAGTGGATAGACAGATGGGGC
TGTTGTTTCAGCTGAGGACACGGTGACCATGGTTCCTGGGCCCCAGAAGT
CAAAGTATCGATAGTTATAGCCATTCAAGCTTGCACAATAATAAGTGGCC
GTGTCCTCAGACCTCAGACTGTTCATTTGCAGGTACAGGGTGCTTTTTGC
ATTATCTCTGGAGATAGTGAATCGGCCCTTCACGGAGTCTCCATAGTAAG
TGTCAGTACCCTCATAACTAATGGATGCGACCCACTCCAGACCCTTCTTT
GGAGACTGGCGGACCCAGGCCATGTAATAGTCACTGAAAGTGAATCCTGA
GGCTGCACAGGAGAGTTTCAGGGACCTTCCAGGCTGCACTAAGCCTCCCC
CAGACTCCACCAGCTGCACCTCACACTGGACACCTTTTATGAAAAGGACA
AGGAAAACCAAGCTGAGCCTGGTGTCCATGGTGAGTAGTCTGTGCAGTGC
TGCAGTACTGATTACTGAATGGGAGAGCCTCAGAGTCCAGGACTGGGCTC
CTGCGTCCGAGGGG

A second set of nested PCR amplification of the variable region of the heavy and light chains was carried out in a reaction master mix; H₂O 50 μl; 5× Phusion™ HF Buffer 10 μl 1×, 10 mM dNTPs 1 μl (200 μM each), primer pairs (for heavy chain M290_VH Forward primer 1 μl (0.5 μM) and M290_VH Reveres primer 1 μl (0.5 μM), (for light chain M290_VL Forward primer 1 μl (0.5 μM) and M290_VL Forward primer 1 μl (0.5 μM), cDNA synthesis reaction mixture 5 μl and Phusion Hot Start DNA Polymerase 0.5 μl (0.02 U/μl).

TABLE 1
primers

| Primer name | Sequence |
|---|---|
| M290_VH | GGCGGAT AGATCT ACTAGT CCCGGG gaggtgcagctggtggag |
| Forward primer | BglII SpeI XmaI binding region |
| | SEQ ID NO: 25 |
| M290_VH | tgaggacacggtgaccatggttcctgggcccca |
| Revere primer | SEQ ID NO: 26 |
| M290_VL | aacatccagatgacccagtctccttcaatactg |
| Forward primer | SEQ ID NO: 27 |
| M290_VL | GCCGCC AAGCTT CTA TGCGGCCGC gaaatgaaacgggctgat |
| Revere primer | HindIII Stop NotI binding region |
| | SEQ ID NO: 28 |

Initial denaturation at 98° C. 30 s, followed by 40 cycles of (denaturation 98° C. 100 s, annealing 65° C. (heavy chain) or 60° C. (light chain) 10 s, and extension 72° C. 40 s).

The generated PCR products were then sequenced to confirm the sequence validity.

>Final VH sequence with restriction site and
primers
(SEQ ID NO: 29) [symbol]
gaggtgcagctggtggagtctgggggaggcttagtgcagcctgg
aaggtccctgaaactctcctgtgcagcctcaggattcactttcagtgact
attacatggcctgggtccgccagtctccaaagaagggtctggagtgggtc
gcatccattagttatgagggtactgacacttactatggagactccgtgaa
gggccgattcactatctccagagataatgcaaaaagcaccctgtacctgc
aaatgaacagtctgaggtctgaggacacggccacttattattgtgcaagc
ttgaatggctataactatcgatactttgacttctggggcccaggaaccat
ggtcaccgtgtcctca
>Final VL sequence with restriction sites and
primers
(SEQ ID NO: 30)
aacatccagatgacccagtctccttcaatactgtctgcatctgtgggaga
cagagtcactctcagctgcaaagcaggtcagaatattaacaattttttag
cctggtatcagcaaaagcttggagaagctcccaaactcctgatatataat
gcaaacagtttggaaacgggcatcccatcaaggttcagtggcagtggatt
tggcacagattacacactcaccatcagcagcctgcggcctgaagatgttg
ccacatatttttgccagcagtataacagttggtacacgtttggaggtggg
accaagctggaaatgaaacgggctgat GCGGCCGCA TAG AAGCTT
GGCGGC

### GS Linker Generation

A nucleotide sequence corresponding to a GS linker was synthesized to form a construct comprising the 15 bp of the end of the VH sequence followed by the GS linker followed by the 15 bp of the beginning of the VL sequence using the following forward and backword primers:

GS-M290 Forward
(SEQ ID NO: 31)
gtc acc gtg tcc tca GGT GGA GGC GGT TCA GGC GGA
GGT GGC TCT GGC GGT GGA GGG AGC GGT GGA GGG AGT
aac atc cag atg acc
GS-M290 Reverse
(SEQ ID NO: 32)
ggt cat ctg gat gtt ACT CCC TCC ACC GCT CCC TCC
ACC GCC AGA GCC ACC TCC GCC TGA ACC GCC TCC ACC
tga gga cac ggt gac

Annealing and amplification using a mixture of the generated VH and VL and the GS linker resulted into the following completed sequence.

Complete CD103 fragment = VH-GS-VL
(SEQ ID NO: 33)
GGCGGATAGATCTACTAGTCCCGGGGAGGTGCAGCTGGTGGAGTCTGGGG
GAGGCTTAGTGCAGCCTGGAAGGTCCCTGAAACTCTCCTGTGCAGCCTCA
GGATTCACTTTCAGTGACTATTACATGGCCTGGGTCCGCCAGTCTCCAAA
GAAGGGTCTGGAGTGGGTCGCATCCATTAGTTATGAGGGTACTGACACTT
ACTATGGAGACTCCGTGAAGGGCCGATTCACTATCTCCAGAGATAATGCA
AAAAGCACCCTGTACCTGCAAATGAACAGTCTGAGGTCTGAGGACACGGC
CACTTATTATTGTGCAAGCTTGAATGGCTATAACTATCGATACTTTGACT
TCTGGGGCCCAGGAACCATGGTCACCGTGTCCTCAGGTGGAGGCGGTTCA
GGCGGAGGTGGCTCTGGCGGTGGAGGGAGCGGTGGAGGGAGTAACATCCA
GATGACCCAGTCTCCTTCAATACTGTCTGCATCTGTGGGAGACAGAGTCA
CTCTCAGCTGCAAAGCAGGTCAGAATATTAACAATTTTTTAGCCTGGTAT
CAGCAAAAGCTTGGAGAAGCTCCCAAACTCCTGATATATAATGCAAACAG
TTTGGAAACGGGCATCCCATCAAGGTTCAGTGGCAGTGGATTTGGCACAG
ATTACACACTCACCATCAGCAGCCTGCGGCCTGAAGATGTTGCCACATAT
TTTTGCCAGCAGTATAACAGTTGGTACACGTTTGGAGGTGGGACCAAGCT
GGAAATGAAACGGGCTGAT GCGGCCGCA TAG AAGCTT GGCGGC
Final core CD103 scFv amino acid sequence
(SEQ ID NO: 16)
PGEVQLVESGGGLVQPGRSLKLSCAASGFTFSDYYMAWVRQSPKKGLEWV
ASISYEGTDTYYGDSVKGRFTISRDNAKSTLYLQMNSLRSEDTATYYCAS
LNGYNYRYFDFWGPGTMVTVSSGGGGSGGGGSGGGGSGGGSNIQMTQSPS
ILSASVGDRVTLSCKAGQNINNFLAWYQQKLGEAPKLLIYNANSLETGIP
SRFSGSGFGTDYTLTISSLRPEDVATYFCQQYNSWYTFGGGTKLEMKRAD
AAA

The core CD103 (M290) scFv above, was used to generate fusion proteins comprising CTA1-X or CTA1 with an Arg7Lys mutation (CTA1(R7K))-X) next to the CD103 scFv sequence followed by His tag and FLAG tag sequences to make the final following DNA sequence products.

### DNA Construct for Bacterial Expression

The following construct was made using the GoldenGate cloning system into a standard pTrc-Bsal E. coli expression vector backbone under control of the P_{Trc} promoter. All sequences were optimized for E. coli expression using GeneArt's tools. Also, a DNA sequence encoding amino acids KN inserted after initial M was included for better protein expression profile.

pTrc-CTA1-3(M2e)-3(Ea)-SIINFEKL-p323-scFvCD103-FLAG-His:
>CTA1 (SEQ ID NO: 34):
GGTCTCCCCATGAAAAATGATGATAAGTTATATCGCGCAGATTCTCGTCCGCCTGATGAAAT
TAAACAGAGCGGTGGTCTGATGCCTCGTGGTCAGAGCGAATATTTTGATCGTGGCACCCAGA
TGAACATCAACCTGTATGATCATGCACGTGGTACACAGACCGGTTTTGTTCGTCATGATGAT
GGTTATGTTAGCACCAGCATTAGCCTGCGTAGCGCACATCTGGTTGGTCAGACCATTCTGAG
CGGTCATAGCACCTATTATATCTATGTTATTGCAACCGCACCGAATATGTTTAACGTTAATG
ATGTTCTGGGTGCCTATAGTCCGCATCCGGATGAACAAGAGGTTAGCGCACTGGGTGGTATT
CCGTATAGCCAGATTTATGGTTGGTATCGTGTTCATTTTGGTGTGCTGGATGAACAGCTGCA
TCGTAATCGTGGTTATCGTGATCGTTATTATAGCAACCTGGATATTGCACCGGCAGCAGATG
GTTATGGTCTGGCAGGTTTTCCGCCTGAACATCGTGCATGGCGTGAAGAACCGTGGATTCAT
CATGCACCGCCTGGTGCAGGTAATGCACCGCGTAGCTCTCGAGACC
>3M2e-3Ea-SIIN-p323 (SEQ ID NO: 35)
GGTCTCCCTCTAGCCTGCTGACCGAAGTTGAAACCCCGATTCGTAATGAATGGGGTAGCCGT
AGCAATGATAGCAGTCTGTTAACGGAAGTGGAAACACCGATCCGCAACGAGTGGGGTAGTCG
TTCAAATGATAGCTCACTGCTGACAGAGGTGGAAACGCCTATAAGAAACGAATGGGGTTCAC
GTAGTAATGATTCAAGCGCAAGCTTTGAAGCACAGGGTGCACTGGCAAATATTGCAGTTGAT
AAAGCAGCCAGTTTTGAGGCCCAAGGTGCCCTGGCCAACATTGCCGTGGACAAAGCAGCGTC
ATTCGAGGCGCAGGGTGCATTAGCCAATATCGCCGTGGATAAGGCAAGCATTATCAACTTTG
AGAAACTGAAAATTAGCCAGGCAGTTCATGCAGCACATGCCGAAATTAATGAAGCAGGTCGA
GCACGAGACC
>scFvCD103-FLAG His (SEQ ID NO: 36):
GGTCTCCAGCAAGTCCGGGTGAAGTTCAGCTGGTTGAAAGCGGTGGTGGTCTGGTTCAGCCT
GGTCGTAGCCTGAAACTGAGCTGTGCAGCAAGCGGTTTTACCTTTAGCGATTATTACATGGC
ATGGGTTCGTCAGAGCCCGAAAAAAGGTCTGGAATGGGTTGCAAGCATTAGCTATGAAGGCA
CCGATACCTATTATGGTGATAGCGTTAAAGGTCGCTTTACCATTAGCCGTGATAATGCAAAA
AGCACCCTGTACCTGCAGATGAATAGCCTGCGTAGCGAAGATACCGCAACCTATTATTGTGC
AAGCCTGAATGGTTATAACTACCGCTATTTTGATTTTTGGGGTCCGGGTACAATGGTTACCG
TTAGCTCAGGTGGTGGTGGTAGCGGTGGCGGTGGTTCTGGTGGTGGCGGATCAGGTGGCGGT
AGCAATATTCAGATGACCCAGAGTCCGAGCATTCTGAGCGCAAGCGTTGGTGATCGTGTTAC
CCTGAGCTGTAAAGCAGGTCAGAATATTAACAATTTCCTGGCATGGTATCAGCAGAAACTGG
GTGAAGCACCGAAACTGCTGATTTATAACGCAAATAGCCTGGAAACCGGTATTCCGAGCCGT
TTTAGCGGTAGCGGTTCTGGCACCGATTATACCCTGACCATTAGCAGCCTGCGTCCGGAAGA
TGTTGCCACCTATTTTTGTCAGCAGTATAATAGCTGGTATACCTTTGGTGCCGGTACAAAAC
TGGAACTGAAACGTGCAGATGCAGCAGCCAAACTGGATTATAAAGATGATGATGATAAGCAC
CATCATCACCACCACTAATAAGCCGAGACC
>pTtc-Bsal (SEQ ID NO: 37):
GGCCGATTCATTAATGCAGCTGGCACGACAGGTTTCCCGACTGGAAAGCGGGCAGTGAGCGC
AACGCAATTAATGTGAGTTAGCGCGAATTGATCTGGTTTGACAGCTTATCATCGACTGCACG
GTGCACCAATGCTTCTGGCGTCAGGCAGCCATCGGAAGCTGTGGTATGGCTGTGCAGGTCGT
AAATCACTGCATAATTCGTGTCGCTCAAGGCGCACTCCCGTTCTGGATAATGTTTTTTGCGC
CGACATCATAACGGTTCTGGCAAATATTCTGAAATGAGCTGTTGACAATTAATCATCCGGCT
CGTATAATGTGTGGAATTGTGAGCGGATAACAATTTCACACAGGAAACAGACCATGGAGACC
TAATAACTGCAGTAATAAGGTCTCTAAGCTTGGCTGTTTTGGCGGATGAGAGAAGATTTTCA
GCCTGATACAGATTAAATCAGAACGCAGAAGCGGTCTGATAAAACAGAATTTGCCTGGCGGC
AGTAGCGCGGTGGTCCCACCTGACCCCATGCCGAACTCAGAAGTGAAACGCCGTAGCGCCGA
TGGTAGTGTGGGATCTCCCCATGCGAGAGTAGGGAACTGCCAGGCATCAAATAAAACGAAAG
GCTCAGTCGAAAGACTGGGCCTTTCGTTTTATCTGTTGTTTGTCGGTGAACGCTCTCCTGAG
TAGGACAAATCCGCCGGGAGCGGATTTGAACGTTGCGAAGCAACGGCCCGGAGGGTGGCGGG
CAGGACGCCCGCCATAAACTGCCAGGCATCAAATTAAGCAGAAGGCCATCCTGACGGATGGC
CTTTTTGCGTTTCTACAAACTCTTTTTGTTTATTTTTCTAAATACATTCAAATATGTATCCG
CTCATGGTGTGTCTCAAAATCTCTGATGTTACATTGCACAAGATAAAAATATATCATCATGA
ACAATAAAACTGTCTGCTTACATAAACAGTAATACAAGGGGTGTTATGAGCCATATTCAACG
GGAAACGTCTTGCTCGAGGCCGCGATTAAATTCCAACATGGATGCTGATTTATATGGGTATA
AATGGGCTCGCGATAATGTCGGGCAATCAGGTGCGACAATCTATCGATTGTATGGGAAGCCC
GATGCGCCAGAGTTGTTTCTGAAACATGGCAAAGGTAGCGTTGCCAATGATGTTACAGATGA
GATGGTCAGACTAAACTGGCTGACGGAATTTATGCCTCTTCCGACCATCAAGCATTTTATCC
GTACTCCTGATGATGCATGGTTACTCACCACTGCGATCCCCGGGAAAACAGCATTCCAGGTA
TTAGAAGAATATCCTGATTCAGGTGAAAATATTGTTGATGCGCTGGCAGTGTTCCTGCGCCG
GTTGCATTCGATTCCTGTTTGTAATTGTCCTTTTAACAGCGATCGCGTATTTCGTCTCGCTC
AGGCGCAATCACGAATGAATAACGGTTTGGTTGATGCGAGTGATTTTGATGACGAGCGTAAT
GGCTGGCCTGTTGAACAAGTCTGGAAAGAAATGCATAAGCTTTTGCCATTCTCACCGGATTC
AGTCGTCACTCATGGTGATTTCTCACTTGATAACCTTATTTTTGACGAGGGGAAATTAATAG
GTTGTATTGATGTTGGACGAGTCGGAATCGCAGACCGATACCAGGATCTTGCCATCCTATGG
AACTGCCTCGGTGAGTTTTCTCCTTCATTACAGAAACGGCTTTTTCAAAAATATGGTATTGA
TAATCCTGATATGAATAAATTGCAGTTTCATTTGATGCTCGATGAGTTTTTCTAATCAGAAT
TGGTTAATTGGTTGTAACACTGGCAGAGCATTACGCTGACTTGACGGGACGGCGGCTTTGTT
GAATAAATCGAACTTTTGCTGAGTTGAAGGATCAGATCACGCATCTTCCCGACAACGCAGAC
CGTTCCGTGGCAAAGCAAAAGTTCAAAATCACCAACTGGTCCCCATGACCAAAATCCCTTAA
CGTGAGTTTTCGTTCCACTGAGCGTCAGACCCCGTAGAAAAGATCAAAGGATCTTCTTGAGA
TCCTTTTTTTCTGCGCGTAATCTGCTGCTTGCAAACAAAAAAACCACCGCTACCAGCGGTGG
TTTGTTTGCCGGATCAAGAGCTACCAACTCTTTTTCCGAAGGTAACTGGCTTCAGCAGAGCG
CAGATACCAAATACTGTCCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAGATA
CCTACAGCGTGAGCTATGAGAAAGCGCCACGCTTCCCGAAGGGAGAAAGGCGGACAGGTATC
CGGTAAGCGGCAGGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATTTTTGTGATGCTC
GTCAGGGGGGCGGAGCCTATGGAAAAACGCCAGCAACGCGGCCTTTTTACGGTTCCTGGCCT
TTTGCTGGCCTTTTGCTCACATGTTCTTTCCTGCGTTATCCCCTGATTCTGTGGATAACCGT
ATTACCGCCTTTGAGTGAGCTGATACCGCTCGCCGCAGCCGAACGACCGAGCGCAGCGAGTC
AGTGAGCGAGGAAGCGGAAGAGCGCCTGATGCGGTATTTTCTCCTTACGCATCTGTGCGGTA
TTTCACACCGCATATGGTGCACTCTCAGTACAATCTGCTCTGATGCCGCATAGTTAAGCCAG
TATACACTCCGCTATCGCTACGTGACTGGGTCATGGCTGCGCCCCGACACCCGCCAACACCC
GCTGACGCGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAGGCAGCAGA
TCAATTCGCGCGCGAAGGCGAAGCGGCATGCATTTACGTTGACACCATCGAATGGTGCAAAA
CCTTTCGCGGTATGGCATGATAGCGCCCGGAAGAGAGTCAATTCAGGGTGGTGAATGTGAAA
CCAGTAACGTTATACGATGTCGCAGAGTATGCCGGTGTCTCTTATCAGACCGTTTCCCGCGT
GGTGAACCAGGCCAGCCACGTTTCTGCGAAAACGCGGGAAAAAGTGGAAGCGGCGATGGCGG
AGCTGAATTACATTCCCAACCGCGTGGCACAACAACTGGCGGGCAAACAGTCGTTGCTGATT
GGCGTTGCCACCTCCAGTCTGGCCCTGCACGCGCCGTCGCAAATTGTCGCGGCGATTAAATC
TCGCGCCGATCAACTGGGTGCCAGCGTGGTGGTGTCGATGGTAGAACGAAGCGGCGTCGAAG
CCTGTAAAGCGGCGGTGCACAATCTTCTCGCGCAACGCGTCAGTGGGCTGATCATTAACTAT
CCGCTGGATGACCAGGATGCCATTGCTGTGGAAGCTGCCTGCACTAATGTTCCGGCGTTATT
TCTTGATGTCTCTGACCAGACACCCATCAACAGTATTATTTTCTCCCATGAAGACGGTACGC
GACTGGGCGTGGAGCATCTGGTCGCATTGGGTCACCAGCAAATCGCGCTGTTAGCGGGCCCA
TTAAGTTCTGTCTCGGCGCGTCTGCGTCTGGCTGGCTGGCATAAATATCTCACTCGCAATCA
AATTCAGCCGATAGCGGAACGGGAAGGCGACTGGAGTGCCATGTCCGGTTTTCAACAAACCA
TGCAAATGCTGAATGAGGGCATCGTTCCCACTGCGATGCTGGTTGCCAACGATCAGATGGCG
CTGGGCGCAATGCGCGCCATTACCGAGTCCGGGCTGCGCGTTGGTGCGGATATCTCGGTAGT
GGGATACGACGATACCGAAGACAGCTCATGTTATATCCCGCCGTCAACCACCATCAAACAGG
ATTTTCGCCTGCTGGGGCAAACCAGCGTGGACCGCTTGCTGCAACTCTCTCAGGGCCAGGCG
GTGAAGGGCAATCAGCTGTTGCCCGTCTCACTGGTGAAAAGAAAAACCACCCTGGCGCCCAA
TACGCAAACCGCCTCTCCCCGCGCGTT

DNA strands according to above were resuspended in H₂O to give concentrations of about 100 ng/μl. These DNA strands plus pTrc-Bsal (pTOL1002) were mixed according to Table 2.

TABLE 2
mixture of ligation reaction

| | | pTrc-CTA1-SIINFEKL- |
|---|---|---|
| | | p323-scFvCD103- |
| Components | Control | FLAG-His |
| Buffer (T4 DNA Ligase) | 2 μL | 2 μL |
| pTrc-Bsal (100 ng/μL) | 1 μL | 1 μL |
| CTA1(100 ng/μL) | 1 μL | 1 μL |
| scFvCD103-FlagHis (100 ng/μL) | | 1 μL |
| 3M2e-3Ea-SIIN-p323 (100 ng/μL) | 1 μL | 1 μL |
| Enz. Mix(Bsal + T4 DNA Ligase) | 1 μL + | 1 μL + |
| | 1 μL | 1 μL |
| H₂O | 14 μL | 13 μL |

The following reaction conditions were used, incubation at: 37° C. 40 min followed by 55° C. 5 min. 2 μl of each reaction was used to transform 20 μl Library Competent DH5a Cells.

The following sequences were synthesized and cloned into the standard pSY-Bsal(5) E. coli expression vector backbones via Aarl cloning sites by Europhingenomics. This resulted in pSY-CTA1-SIINFEKL(I)-P232(II)-CD103 scFv-FLAG tag-His tag, CTA1-P232(II)-CD103 scFv-FLAG tag-His tag and pSY-CTA1(R7k)-P323-CD103 scFv-FLAG tag-His tag. All sequences were optimized for E. coli expression using GeneArt's tools.

CTA1-SIINFEKL(I)-P232(II)-CD103 scFv-FLAG tag-His tag
(SEQ ID NO: 38)
ATCACCTGCATACCATGGATGATAAGTTATATCGGGCAGATTCTAGACCTCCTGATGAAATA
AAGCAGTCAGGTGGTCTTATGCCAAGAGGACAGAGTGAGTACTTTGACCGAGGTACTCAAAT
GAATATCAACCTTTATGATCATGCAAGAGGAACTCAGACGGGATTTGTTAGGCACGATGATG
GATATGTTTCCACCTCAATTAGTTTGAGAAGTGCCCACTTAGTGGGTCAAACTATATTGTCT
GGTCATTCTACTTATTATATATATGTTATAGCCACTGCACCCAACATGTTTAACGTTAATGA
TGTATTAGGGGCATACAGTCCTCATCCAGATGAACAAGAAGTTTCTGCTTTAGGTGGGATTC
CATACTCCCAAATATATGGATGGTATCGAGTTCATTTTGGGGTGCTTGATGAACAATTACAT
CGTAATAGGGGCTACAGAGATAGATATTACAGTAACTTAGATATTGCTCCAGCAGCAGATGG
TTATGGATTGGCAGGTTTCCCTCCGGAGCATAGAGCTTGGAGGGAAGAGCCGTGGATTCATC
ATGCACCGCCGGGTTGTGGGAATGCTCCAAGATCATCGGGATCTACTAGTAGTATAATCAAC
TTTGAAAAACTGATCTCCCAGGCTGTTCACGCTGCTCACGCTGAAATCAACGAAGCTGGTCG
TGCCAGATCTCCCGGGGAGGTGCAGCTGGTGGAGTCTGGGGGAGGCTTAGTGCAGCCTGGAA
GGTCCCTGAAACTCTCCTGTGCAGCCTCAGGATTCACTTTCAGTGACTATTACATGGCCTGG
GTCCGCCAGTCTCCAAAGAAGGGTCTGGAGTGGGTCGCATCCATTAGTTATGAGGGTACTGA
CACTTACTATGGAGACTCCGTGAAGGGCCGATTCACTATCTCCAGAGATAATGCAAAAAGCA
CCCTGTACCTGCAAATGAACAGTCTGAGGTCTGAGGACACGGCCACTTATTATTGTGCAAGC
TTGAATGGCTATAACTATCGATACTTTGACTTCTGGGGCCCAGGAACCATGGTCACCGTGTC
CTCAGGTGGAGGCGGTTCAGGCGGAGGTGGCTCTGGCGGTGGAGGGAGCGGTGGAGGGAGTA
ACATCCAGATGACCCAGTCTCCTTCAATACTGTCTGCATCTGTGGGAGACAGAGTCACTCTC
AGCTGCAAAGCAGGTCAGAATATTAACAATTTCTTAGCCTGGTATCAGCAAAAGCTTGGAGA
AGCTCCCAAACTCCTGATATATAATGCAAACAGTTTGGAAACGGGCATCCCATCAAGGTTCA
GTGGCAGTGGATCTGGCACAGATTACACACTCACCATCAGCAGCCTGCGGCCTGAAGATGTT
GCCACATATTTCTGCCAGCAGTATAACAGTTGGTACACGTTTGGAGCTGGGACCAAGCTGGA
ACTGAAACGGGCTGATGCGGCCGCAAAGCTTGATTACAAGGATGACGATGACAAGCATCACC
ATCATCACCATTAGAGCTTCGCAGGTGCTTC
CTA1-P232(II)-CD103 scFv-FLAG tag-His tag
(SEQ ID NO: 39)
ATCACCTGCATACCATGGATGGATGATAAGTTATATAAGGCAGATTCTAGACCTCCTGATGA
AATAAAGCAGTCAGGTGGTCTTATGCCAAGAGGACAGAGTGAGTACTTTGACCGAGGTACTC
AAATGAATATCAACCTTTATGATCATGCAAGAGGAACTCAGACGGGATTTGTTAGGCACGAT
GATGGATATGTTTCCACCTCAATTAGTTTGAGAAGTGCCCACTTAGTGGGTCAAACTATATT
GTCTGGTCATTCTACTTATTATATATATGTTATAGCCACTGCACCCAACATGTTTAACGTTA
ATGATGTATTAGGGGCATACAGTCCTCATCCAGATGAACAAGAAGTTTCTGCTTTAGGTGGG
ATTCCATACTCCCAAATATATGGATGGTATCGAGTTCATTTTGGGGTGCTTGATGAACAATT
ACATCGTAATAGGGGCTACAGAGATAGATATTACAGTAACTTAGATATTGCTCCAGCAGCAG
ATGGTTATGGATTGGCAGGTTTCCCTCCGGAGCATAGAGCTTGGAGGGAAGAGCCGTGGATT
CATCATGCACCGCCGGGTTGTGGGAATGCTCCAAGATCATCGGGATCTACTAGTATCTCCCA
GGCTGTTCACGCTGCTCACGCTGAAATCAACGAAGCTGGTCGTGCCAGATCTCCCGGGGAGG
TGCAGCTGGTGGAGTCTGGGGGAGGCTTAGTGCAGCCTGGAAGGTCCCTGAAACTCTCCTGT
GCAGCCTCAGGATTCACTTTCAGTGACTATTACATGGCCTGGGTCCGCCAGTCTCCAAAGAA
GGGTCTGGAGTGGGTCGCATCCATTAGTTATGAGGGTACTGACACTTACTATGGAGACTCCG
TGAAGGGCCGATTCACTATCTCCAGAGATAATGCAAAAAGCACCCTGTACCTGCAAATGAAC
AGTCTGAGGTCTGAGGACACGGCCACTTATTATTGTGCAAGCTTGAATGGCTATAACTATCG
ATACTTTGACTTCTGGGGCCCAGGAACCATGGTCACCGTGTCCTCAGGTGGAGGCGGTTCAG
GCGGAGGTGGCTCTGGCGGTGGAGGGAGCGGTGGAGGGAGTAACATCCAGATGACCCAGTCT
CCTTCAATACTGTCTGCATCTGTGGGAGACAGAGTCACTCTCAGCTGCAAAGCAGGTCAGAA
TATTAACAATTTTTTAGCCTGGTATCAGCAAAAGCTTGGAGAAGCTCCCAAACTCCTGATAT
ATAATGCAAACAGTTTGGAAACGGGCATCCCATCAAGGTTCAGTGGCAGTGGATTTGGCACA
GATTACACACTCACCATCAGCAGCCTGCGGCCTGAAGATGTTGCCACATATTTTTGCCAGCA
GTATAACAGTTGGTACACGTTTGGAGGTGGGACCAAGCTGGAAATGAAACGGGCTGATGCGG
CCGCAAAGCTTGATTACAAGGATGACGATGACAAGCATCACCATCATCACCATTAGAGCTTC
GCAGGTGCTTC
CTA1(R7k)-P323- CD103 scFv-FLAG tag-His tag
(SEQ ID NO: 40)
ATCACCTGCATACCATGGATGGATGATAAGTTATATAAGGCAGATTCTAGACCTCCTGATGA
AATAAAGCAGTCAGGTGGTCTTATGCCAAGAGGACAGAGTGAGTACTTTGACCGAGGTACTC
AAATGAATATCAACCTTTATGATCATGCAAGAGGAACTCAGACGGGATTTGTTAGGCACGAT
GATGGATATGTTTCCACCTCAATTAGTTTGAGAAGTGCCCACTTAGTGGGTCAAACTATATT
GTCTGGTCATTCTACTTATTATATATATGTTATAGCCACTGCACCCAACATGTTTAACGTTA
ATGATGTATTAGGGGCATACAGTCCTCATCCAGATGAACAAGAAGTTTCTGCTTTAGGTGGG
ATTCCATACTCCCAAATATATGGATGGTATCGAGTTCATTTTGGGGTGCTTGATGAACAATT
ACATCGTAATAGGGGCTACAGAGATAGATATTACAGTAACTTAGATATTGCTCCAGCAGCAG
ATGGTTATGGATTGGCAGGTTTCCCTCCGGAGCATAGAGCTTGGAGGGAAGAGCCGTGGATT
CATCATGCACCGCCGGGTTGTGGGAATGCTCCAAGATCATCGGGATCTACTAGTATCTCCCA
GGCTGTTCACGCTGCTCACGCTGAAATCAACGAAGCTGGTCGTGCCAGATCTCCCGGGGAGG
TGCAGCTGGTGGAGTCTGGGGGAGGCTTAGTGCAGCCTGGAAGGTCCCTGAAACTCTCCTGT
GCAGCCTCAGGATTCACTTTCAGTGACTATTACATGGCCTGGGTCCGCCAGTCTCCAAAGAA
GGGTCTGGAGTGGGTCGCATCCATTAGTTATGAGGGTACTGACACTTACTATGGAGACTCCG
TGAAGGGCCGATTCACTATCTCCAGAGATAATGCAAAAAGCACCCTGTACCTGCAAATGAAC
AGTCTGAGGTCTGAGGACACGGCCACTTATTATTGTGCAAGCTTGAATGGCTATAACTATCG
ATACTTTGACTTCTGGGGCCCAGGAACCATGGTCACCGTGTCCTCAGGTGGAGGCGGTTCAG
GCGGAGGTGGCTCTGGCGGTGGAGGGAGCGGTGGAGGGAGTAACATCCAGATGACCCAGTCT
CCTTCAATACTGTCTGCATCTGTGGGAGACAGAGTCACTCTCAGCTGCAAAGCAGGTCAGAA
TATTAACAATTTTTTAGCCTGGTATCAGCAAAAGCTTGGAGAAGCTCCCAAACTCCTGATAT
ATAATGCAAACAGTTTGGAAACGGGCATCCCATCAAGGTTCAGTGGCAGTGGATTTGGCACA
GATTACACACTCACCATCAGCAGCCTGCGGCCTGAAGATGTTGCCACATATTTTTGCCAGCA
GTATAACAGTTGGTACACGTTTGGAGGTGGGACCAAGCTGGAAATGAAACGGGCTGATGCGG
CCGCAAAGCTTGATTACAAGGATGACGATGACAAGCATCACCATCATCACCATTAGAGCTTC
GCAGGTGCTTC
pSY-AaRI
(SEQ ID NO: 41)
CTCCAAAAGGAGCCTTTAATTGTATCGGTTTATCAGCTTGCTTTCGAGGTGAATTTCGACCT
CTAGACCACGCTTGGCTGCAGGTCGACGGATCTCGATCCCGCGAAATTAATACGACTCACTA
TAGGGAGACCACAACGGTTTCCCTCTAGAAATAATTTTGTTTAACTTTAAGAAGGAGATATA
CCATGGATGGCAGGTGCTATAAGTTAATCATCACCACCTGCCATTAGAGCTTCTCAAATAAG
ATGGTCCCATAGTCTGTATCCAAATAATGAATCTTCGGGTGTTTCCCTTTAGCTAAGCACAG
ATCCGGCTGCTAACAAAGCCCGAAAGGAAGCTGAGTTGGCTGCTGCCACCGCTGAGCAATAA
CTAGCATAACCCCTTGGGGCCTCTAAACGGGTCTTGAGGGGTTTTTTGCTGAAAGGAGGAAC
TATATCCGGATGGGAATTCCCCGCGCGCGATGCCCTTTCGTCTTCGAATAAATACCTGTGAC
GGAAGATCACTTCGCAGAATAAATAAATCCTGGTGTCCCTGTTGATACCGGGAAGCCCTGGG
CCAACTTTTGGCGAAAATGAGACGTTGATCGGCACGTAAGAGGTTCCAACTTTCACCATAAT
GAAATAAGATCACTACCGGGCGTATTTTTTGAGTTATCGAGATTTTCAGGAGCTAAGGAAGC
TAAAATGGAGAAAAAAATCACTGGATATACCACCGTTGATATATCCCAATGGCATCGTAAAG
AACATTTTGAGGCATTTCAGTCAGTTGCTCAATGTACCTATAACCAGACCGTTCAGCTGGAT
ATTACGGCCTTTTTAAAGACCGTAAAGAAAAATAAGCACAAGTTTTATCCGGCCTTTATTCA
CATTCTTGCCCGCCTGATGAATGCTCATCCGGAATTTCGTATGGCAATGAAAGACGGTGAGC
TGGTGATATGGGATAGTGTTCACCCTTGTTACACCGTTTTCCATGAGCAAACTGAAACGTTT
TCATCGCTCTGGAGTGAATACCACGACGATTTCCGGCAGTTTCTACACATATATTCGCAAGA
TGTGGCGTGTTACGGTGAAAACCTGGCCTATTTCCCTAAAGGGTTTATTGAGAATATGTTTT
TCGTCTCAGCCAATCCCTGGGTGAGTTTCACCAGTTTTGATTTAAACGTGGCGAATATGGAC
AACTTCTTCGCCCCCGTTTTCACTATGGGCAAATATTATACGCAAGGCGACAAGGTGCTGAT
GCCGCTGGCGATTCAGGTTCATCATGCCGTTTGTGATGGCTTCCATGTCGGCAGAATGCTTA
ATGAATTACAACAGTACTGCGATGAGTGGCAGGGCGGGGCGTAATTTTTTTAAGGCAGTTAT
TGGTGCCCTTAAACGCCTGGTTGCTACGCCTGAATAAGTGATAATAAGCGGATGAATGGCAG
AAATTCGAGCCCGCCTAATGAGCGGGCTTTTTTTTAGCCCGCCTAATGAGCGGGCTTTTTTT
TCGAAAGCAAATTCGACCCATCGCGCGCGGGGAGTCAACTCAGCAAAAGTTCGATTTATTCA
ACAAAGCCACGTTGTGTCTCAAAATCTCTGATGTTACATTGCACAAGATAAAAATATATCAT
CATGAACAATAAAACTGTCTGCTTACATAATATTGAAAAAGGAAGAGTATGAGTATTCAACA
TTTCCGTGTCGCCCTTATTCCCTTTTTTGCGGCATTTTGCCTTCCTGTTTTTGCTCACCCAG
AAACGCTGGTGAAAGTAAAAGATGCTGAAGATCAGTTGGGTGCACGAGTGGGTTACATCGAA
CTGGATCTCAACAGCGGTAAGATCCTTGAGAGTTTTCGCCCCGAAGAACGTTTTCCAATGAT
GAGCACTTTTAAAGTTCTGCTATGTGGCGCGGTATTATCCCGTGTTGACGCCGGGCAAGAGC
AACTCGGTCGCCGCATACACTATTCTCAGAATGACTTGGTTGAGTACTCACCAGTCACAGAA
AAGCATCTTACGGATGGCATGACAGTAAGAGAATTATGCAGTGCTGCCATAACCATGAGTGA
TAACACTGCGGCCAACTTACTTCTGACAACGATCGGAGGACCGAAGGAGCTAACCGCTTTTT
TGCACAACATGGGGGATCATGTAACTCGCCTTGATCGTTGGGAACCGGAGCTGAATGAAGCC
ATACCAAACGACGAGCGTGACACCACGATGCCTGCAGCAATGGCAACAACGTTGCGCAAACT
ATTAACTGGCGAACTACTTACTCTAGCTTCCCGGCAACAATTAATAGACTGGATGGAGGCGG
ATAAAGTTGCAGGACCACTTCTGCGCTCGGCCCTTCCGGCTGGCTGGTTTATTGCTGATAAA
TCTGGAGCCGGTGAGCGTGGGTCTCGCGGTATCATTGCAGCACTGGGGCCAGATGGTAAGCC
CTCCCGTATCGTAGTTATCTACACGACGGGGAGTCAGGCAACTATGGATGAACGAAATAGAC
AGATCGCTGAGATAGGTGCCTCACTGATTAAGCATTGGTAACTGTCAGACCAAGTTTACTCA
TATATACTTTAGATTGATTTAAAACTTCATTTTTAATTTAAAAGGATCTAGGTGAAGATCCT
TTTTGATAATCTCATGACCAAAATCCCTTAACGTGAGTTTTCGTTCCACTGAGCGTCAGACC
CCGTAGAAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTACCAACTCT
TTTTCCGAAGGTAACTGGCTTCAGCAGAGCGCAGATACCAAATACTGTCCTTCTAGTGTAGC
CGTAGTTAGGCCACCACTTCAAGAACTCTGTAGCACCGCCTACATACCTCGCTCTGCTAATC
CTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCT
TGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGTTCTTTCC
TGCGTTATCCCCTGATTCTGTGGATAACCGTATTACCGCCTTTGAGTGAGCTGATACCGCTC
GCCGCAGCCGAACGACCGAGCGCAGATCAAATTGTAAACGTTAATATTTTGTTAAAATTCGC
GTTAAATTTTTGTTAAATCAGCTCATTTTTTAACCAATAGGCCGAAATCGGCAAAATCCCTT
ATAAATCAAAAGAATAGACCGAGATAGGGTTGAGTGTTGTTCCAGTTTGGAACAAGAGTCCA
CTATTAAAGAACGTGGACTCCAACGTCAAAGGGCGAAAAACCGTCTATCAGGGCGATGGCCC
ACTACGTGAACCATCACCCTAATCAAGTTTTTTGGGGTCGAGGTGCCGTAAAGCACTAAATC
GGAACCCTAAAGGGAGCCCCCGATTTAGAGCTTGACGGGGAAAGCCGGCGAACGTGGCGAGA
AAGGAAGGGAAGAAAGCGAAAGGAGCGGGCGCTAGGGCGCTGGCAAGTGTAGCGGTCACGCT
GCGCGTAACCACCACACCCGCCGCGCTTAATGCGCCGCTACAGGGCGCGTGATCTGCATCCG
CTTACAGACAAGCTGTGACCGTCTCCGGGAGCTGCATGTGTCAGAGGTTTTCACCGTCATCA
CCGAAACGCGCGAGGCAGAGCTTGGAATTGCGAATAATAATTTTTTCACGTTGAAAATCTCC
AAAAAAAAAGGCTCCAAAAGGAGCCTTTAATTGTATCGGTTTATCAGCTTGCTTTCGAGGTG
AATTAGCTTGGAATTGCGAATAATAATTTTTTCACGTTGAAAATCTCCAAAAAAAAAGG

### DNA Construction Design for Expression in Insect Cell

Eukaryoutic expression of the fusion protein was performed in baculovirus Expression (BEV) system (Invitrogen) according to the protocol Bac-to-Bac Baculovirus Expression System Publication Number MAN0000414. Briefly, MSS-CTA1-3(M2e)-3(Ea)-CD103 scFv-Flag tag-His tag sequence starting by 63 bp Melittin Signal Sequence (MSS) was synthesized and cloned under polyhydrin promoter (P_{PH}) BamHI and KpnI sites into the pFASTBAC1 baculovirus standard expression shuttle vector by Europhingenomics.

mSS-CTA1-3(M2e)-3(Ea)-CD103 scFv-FLAG tag-His tag
(SEQ ID NO: 42)
GCGCGGATCCATGAAATTTTTGGTGAACGTGGCCTTGGTGTTTATGGTGG
TGTACATTTCGTACATTTACGCCGACCCGTCGCCGGACGACAAATTGTAC
AGAGCCGACTCGAGACCGCCGGACGAAATTAAACAATCGGGCGGCTTGAT
GCCGAGAGGCCAATCGGAATACTTTGACAGAGGCACGCAAATGAACATTA
ACTTGTACGACCACGCCAGAGGCACGCAAACGGGCTTTGTGAGACACGAC
GACGGCTACGTGTCGACGTCGATTTCGTTGAGATCGGCCCACTTGGTGGG
CCAAACGATTTTGTCGGGCCACTCGACGTACTACATTTACGTGATTGCCA
CGGCCCCGAACATGTTTAACGTGAACGACGTGTTGGGCGCCTACTCGCCG
CACCCGGACGAACAAGAAGTGTCGGCCTTGGGCGGCATTCCGTACTCGCA
AATTTACGGCTGGTACAGAGTGCACTTTGGCGTGTTGGACGAACAATTGC
ACAGAAACAGAGGCTACAGAGACAGATACTACTCGAACTTGGACATTGCC
CCGGCCGCCGACGGCTACGGCTTGGCCGGCTTTCCGCCGGAACACAGAGC
CTGGAGAGAAGAACCGTGGATTCACCACGCCCCGCCGGGCTGTGGCAACG
CCCCGAGATCGTCGGGGAATTCGTTGTTGACGGAAGTGGAAACGCCGATT
AGAAACGAATGGGGCTCGAGATCGAACGACTCGTCGTTGTTGACGGAAGT
GGAAACGCCGATTAGAAACGAATGGGGCTCGAGATCGAACGACTCGTCGT
TGTTGACGGAAGTGGAAACGCCGATTAGAAACGAATGGGGCTCGAGATCG
AACGACTCGTCGGACGGTTTAATTAAGTTTAAACCATATGAAGCCAGCTT
CGAGGCCCAGGGCGCCCTGGCCAACATCGCCGTGGACAAGGCCGCCAGCT
TCGAGGCCCAGGGCGCCCTGGCCAACATCGCCGTGGACAAGGCCGCCAGC
TTCGAGGCCCAGGGCGCCCTGGCCAACATCGCCGTGGACAAGGCCAGTTT
AAACCCCGGGAGCGGGGAGGTGCAGCTGGTGGAGTCTGGGGGAGGCTTAG
TGCAGCCTGGAAGGTCCCTGAAACTCTCCTGTGCAGCCTCAGGATTCACT
TTCAGTGACTATTACATGGCCTGGGTCCGCCAGTCTCCAAAGAAGGGTCT
GGAGTGGGTCGCATCCATTAGTTATGAGGGTACTGACACTTACTATGGAG
ACTCCGTGAAGGGCCGATTCACTATCTCCAGAGATAATGCAAAAAGCACC
CTGTACCTGCAAATGAACAGTCTGAGGICTGAGGACACGGCCACTTATTA
TTGTGCAAGCTTGAATGGCTATAACTATCGATACTTTGACTTCTGGGGCC
CAGGAACCATGGTCACCGTGTCCTCAGGTGGAGGCGGTTCAGGCGGAGGT
GGCTCTGGCGGTGGAGGGAGCGGTGGAGGGAGTAACATCCAGATGACCCA
GTCTCCTTCAATACTGTCTGCATCTGTGGGAGACAGAGTCACTCTCAGCT
GCAAAGCAGGTCAGAATATTAACAATTTTTTAGCCTGGTATCAGCAAAAG
CTTGGAGAAGCTCCCAAACTCCTGATATATAATGCAAACAGTTTGGAAAC
GGGCATCCCATCAAGGTTCAGTGGCAGTGGATTTGGCACAGATTACACAC
TCACCATCAGCAGCCTGCGGCCTGAAGATGTTGCCACATATTTTTGCCAG
CAGTATAACAGTTGGTACACGTTTGGAGGTGGGACCAAGCTGGAAATGAA
ACGGGCTGATGCGGCCGCATATTCGAAGAGCTCCGACTACAAAGACGACG
ACGACAAACACCACCACCACCACCACTAGGGTACCAA

The Generated pFASTBAC1-CTA1-3(M2e)-3(Ea)-CD103 scFv-FLAG tag-His tag donor plasmid was transformed into standard DH5a E. coli, amplified and purified using a Qiagen mini plasmid purification kit (Qiagen). pFASTBAC1-CTA1-3(M2e)-3(Ea)-CD103 scFv-FLAG tag-His donor plasmid was then used to transfer CTA1-3(M2e)-3(Ea)-CD103 scFv-FLAG tag into baculovirus genom (Bacmid) into MAX efficiency DH10 BAC E. coli containing Bacmid in site specific transposition event known as a BAC-TO-BAC. The bacmids were purified from DH10Bac single clonies and confirmed for carrying CTA1-3(M2e)-3(Ea)-CD103 scFv-FLAG tag gene by having band of 4163 bp for specific PCR with PUC/M13 forward and reverse primer according to protocols. The extracted recombinant bacmid was purified using HiPure Plasmid Miniprep Kit (Invitrogen) and used in the next step for recombinant Baculo vector production and protein expression. The following mature protein sequence with the weight (64.43 KDa) was expected to be expressed after SS cleavage (according to prediction by SignalP 5.0), (SEQ ID NO: 43):

DPSPDDKLYRADSRPPDEIKQSGGLMPRGQSEYFDRGTQMNINLYDHARG
TQTGFVRHDDGYVSTSISLRSAHLVGQTILSGHSTYYIYVIATAPNMFNV
NDVLGAYSPHPDEQEVSALGGIPYSQTYGWYRVHFGVLDEQLHRNRGYRD
RYYSNLDIAPAADGYGLAGFPPEHRAWREEPWIHHAPPGCGNAPRSSGNS
LLTEVETPIRNEWGSRSNDSSLLTEVETPIRNEWGSRSNDSSLLTEVETP
IRNEWGSRSNDSSDGLIKFKPYEASFEAQGALANIAVDKAASFEAQGALA
NIAVDKAASFKKGLEWVASISYEGTDTYYGDSVKGRFTISRDNAKSTLYL
QMNSLRSEDTATYYCASLNGYNYRYFDFWGPGTMVTVSSGGGGSGGGGSG
GGGSGGGSNIQMTQSPSILSASVGDRVTLSCKAGQNINNFLAWYQQKLGE
APKLLIYNANSLETGIPSRFSGSGFGTDYTLTISSLRPEDVATYFCQQYN
SWYTFGGGTKLEMKRADAAAYSKSSDYKDDDDKHHHHHH

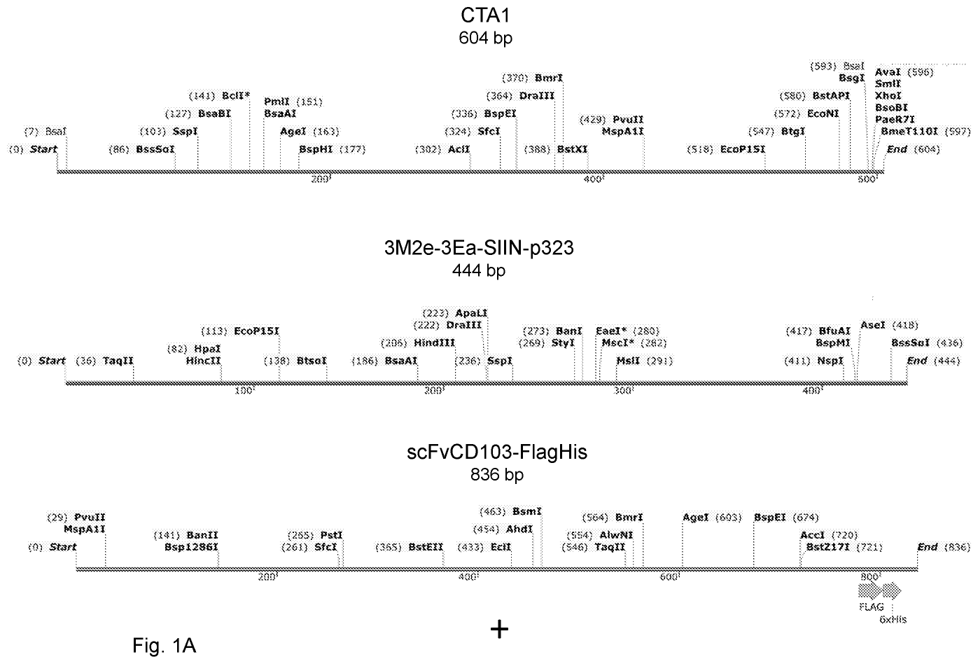

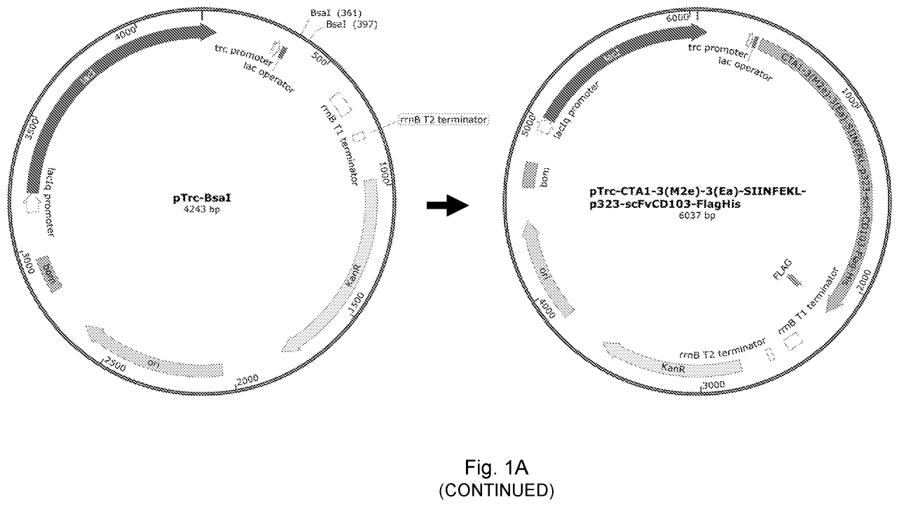

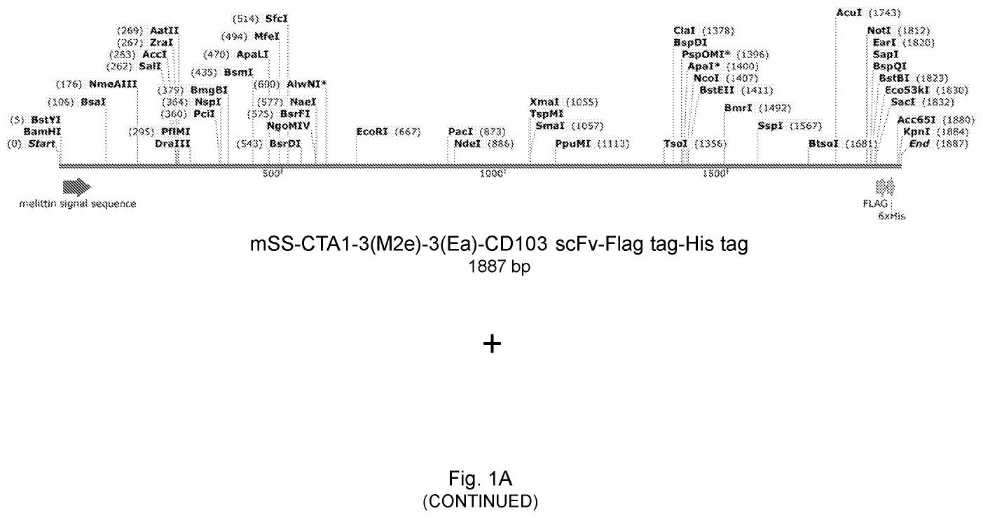

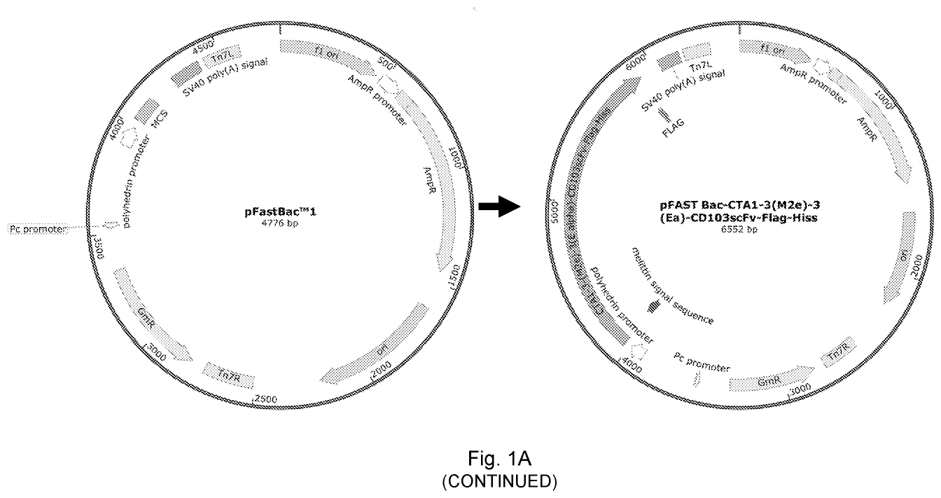

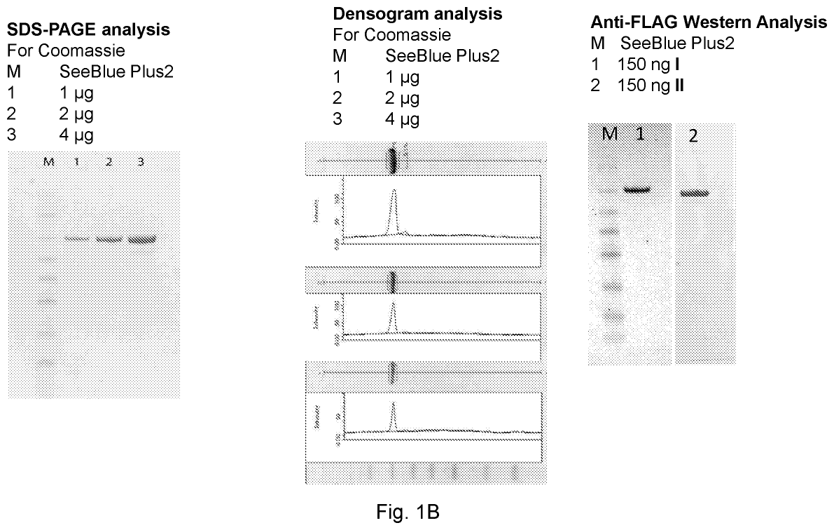

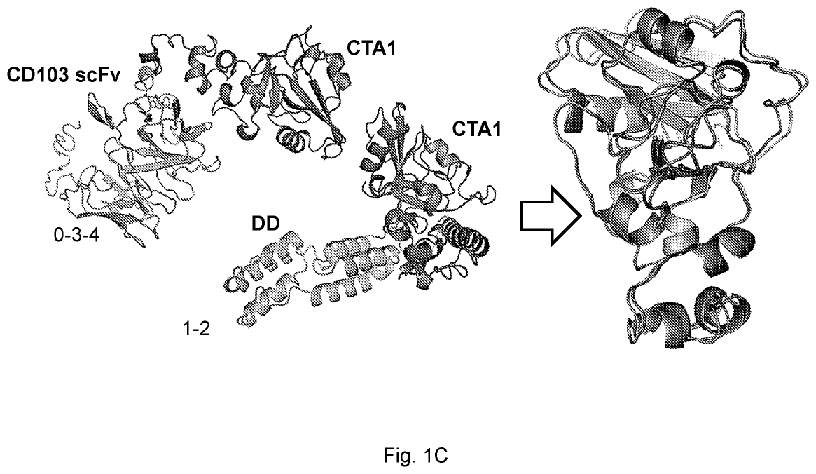

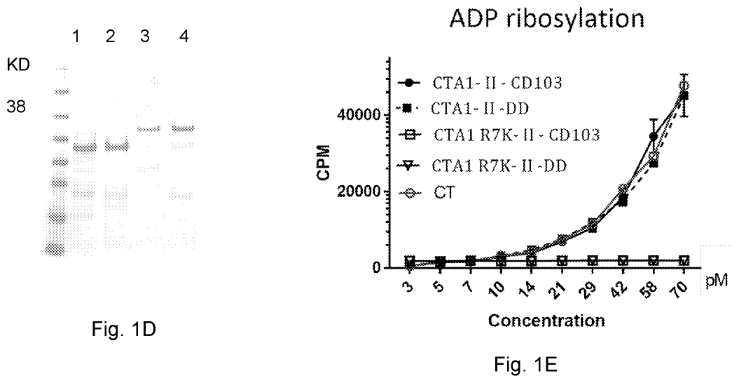

FIG. 1 A is a schematic representation of in silico cloning strategy of the adjuvant active fusion proteins; CTA1-3M2e-3Eα-SIINFEKL-P323-CD103-FLAG-His in prokaryoutic (E. coli) expression system and CTA1-3M2e-3Eα-SIINFEKL-P323-CD103-FLAG-His in eukaryoutic system (Baculovirus).

### Protein Expression Step

### Prokaryoutic System

pTcr constructs were transformed and freezed in E. coli DH5a from a glycerol stock. Inoculated 50 ml 2×YT (+50 μl Kanamycin (1 μg/μL)) in 500 ml shaker flasks with CTA1-3M2e-3Eα-SIINFEKL-Ova-CD103-FLAG-His in DH5a in glycerol. Incubated at 37° C. 260 rpm over night. 2×1 L SYPPG Media+1 ml Kanamsycin (100 mg/ml) were inoculated with seed culture in 5 L shaker flasks with the ratio of 1/10. 2 hours after incubation at 37° C. 260 rpm, the process proceed by incubation with 1 ml 1M IPTG/L. The cultures were kept over night and then harvested by centrifugation at 7000×g (Beckman Coulter Avanti J-20 XP, rotor JLA 8.1000) at 4° C. for 25 min. The pellets were transferred to 50 ml falcon tubes and stored at −20° C. until lysis, solubilization and refording step.

pSY constructs were transformed and freezed in E. coli BL21 from a glycerol stock, LB+Cb. Colonies from the glycerol stock were transferred to 50 mL 2×YT media+Cb, 100 μg/L and incubated in a shaker at 37° C. for 7.5 hours and then stored over night at 4° C. 2 L of 2×YT media+AMP, 100 μg/m L in flasks was inoculated with 16 mL/2 L from the preculture and incubated in a shaker at 37° C. The cultures were harvested by centrifugation at 7000×g (Beckman Coulter Avanti J-20 XP, rotor JLA 8.1000) at 4° C. for 25 min. The supernatant was discarded and the pellets were collected and frozen at −20° C. until lysis, solubilization and refording step.

### Eukaryotic System

Recombinant Baculo Virus was generated according to System BEV Publication Number MAN0000414 protocol. Briefly, 8×10⁵ adherent Spodoptra feragipedra (SF9) cells (viability>98%) in mid log phase growth were plated in a well of 6 well culture plat (nunc) and transfected with 500 ng of purified recombinant bacmid using cellfectin II transfection reagent in non supplemented Grace medium. Grace medium was replaced by serum free medium SF-900 II (Invitrogen cat #10902-088) and the plate was incubated at 27° C. with good ariation. Cells were investigated 72 hours later after transfection for virus specific CytoPathic Effects (CPE). Supernatant from CPE positive cultures were harvested and virus titer was determined by plaque assay to be 5×10⁶ Plaque Forming Unit (PFU)/mL. The supernatant was used as 1^{th} generation of recombinant baculovirus vector seed (P1). 2 mL of P1 was used to transduce fresh mid log phase SF9 2×10⁶ cells/mL in 50 mL suspension culture in a Baffled Bottom 125 mL corning vent cap erlenmeyer cell culture flasks (Sigma Aldright) at 100 rpm speed. The final inoculum volume to reach Multiplicity Of Infection (MOI)=0.1 for producing 2^{nd} generation of virus stock seed (P2) calculated using the following equation:

$$Inoculom required ( mL ) = ( \frac{MOI ( pfu / cell ) * number of cells}{( pfu / mL ) s tock viral titer} )$$

P2 titer was determined to be 10⁸ PFU/mL. To express CTA1-3(M2e)-3(Ea)-CD103 scFv-FLAG tag-His tag, 1 L of High Five insect cells (1×10⁶ cell/mL) (viability>98%) in mid log phase were grown in serum free Express Five medium (Invitrogen) supplemented with 5 μg/mL E-64 cystein inhibitor (Selecchem cat #S7379) in a Baffled Bottom 2 L corning vent cap Erlenmeyer cell culture flasks (Sigma Aldright). 10 mL of P2 inoculom was added to the culture and incubated at 27° C. with good ariation and 80 rpm speed. 72 hours after viral vector inoculation, the culture was harvest by centrifugation at 560×g (Beckman Coulter Avanti J-20 XP, rotor JLA 8.1000) at 4° C. for 15 min. The supernatant was separated and centrifuged at 7000×g (Beckman Coulter Avanti J-20 XP, rotor JLA 8.1000) at 4° C. for 25 min. The supernatant was separated and proceed to purification step.

### Lysis, Solubilization and Refording Step

### Lysis

The pellets were resuspended in 120 ml Lysis buffer (0.1M Tris, 0.1M NaCl pH7.4)+120 μl lysozyme (30 mg/ml) and incubated at 15 min at room temperature in a beaker with stirring at 250 rpm. The cell re-suspension was split into 4×30 ml into Oakridge tubes. Each tube was sonicated 3×2 min (15 s on 5 s off, 70% Amplitude) on ice. The tubes were centrifuged JA25.50, 22000×g, 30 min, 4° C. Supernatants were discarded and pellets were stored +4° C.

### Wash

Each pellet was resuspended in 30 ml Wash buffer (0.1 M Tris, 0.1 M NaCl, 2 M Urea, 2% Triton X-100 pH 7.4)−4×30 ml total and sonicated 3×2 min (15 s on, 5 s off, 65% Amplitude) on ice. The tubes were centrifuged JA25.50, 22000×g, 30 min, 4° C. and the supernatants were discarded. The wash procedure was performed three times, and the final pellets were stored at −20° C.

### Solubilisation

One pellet from the lysis preparation was resuspended in 30 ml total Solubilisation buffer (20 mM Tris, 6 M guanidinium HCl, 0.15 M NaCl, pH 7.4) and left in room temperature for 10 min, and sonicated 2 min (15 s on, 5 s off, 65% Amplitude) on ice. The tube was then left in room temperature for 2 hours and 20 min followed by centrifugation JA25.50, 38000×g, 1 h 30 min, 20° C. The supernatant was transferred to 50 ml Falcon tube and the solubilised protein concentration was determined.

### Refolding

Solubilised protein was added dropwise to 2 L Refolding buffer (20 mM Tris, 2 M Urea, 10% Glycerol, 5% Sucrose, 20 mM Imidazole pH 7.4) using a peristaltic pump (Speed 0.5 1× flow rate) at ≈6° C. Buffer was stirred slowly for 6 hours. After addition of buffer to allow refolding, the fusion protein was purified by affinity chromatography using a His tag affinity column in the following steps.

### Purification Step

Refolded protein was loaded overnight onto HisTrapFF crude 5 ml column equilibrated with buffer A (20 mM Tris, 6 M Guanidinium HCl, 0.15 M NaCl, 20 mM Imidazole pH 7.4). The protein was eluted in steps between buffer A (20 mM NaP, 0.5 M NaCl, 30 mM Imidazole pH 7.4) and buffer B (20 mM NaP, 0.5 M NaCl, 500 mM Imidazole pH 7.4).

Superdex 200 column purification by 350 ml SEC Buffer (10 mM NaP, 160 mM NaCl pH 7.4) at a flow 2.5 ml/min. Then sample loaded into the superloop with following method:
1. 0.00 Base Volume
2. 0.00 Gradient 0.00 {% B} 0.00 {base}
3. 0.00 Flow 2.5 {ml/min}
4. 0.00 InjectionValve Inject
5. 12.00 InjectionValve Load
6. 350.00 End_Method

The purity of the purified fusion proteins was determined by SDS-PAGE to be between 93-97%. The identity of the fusion protein was confirmed by Western blot AP anti-FLAG tag (Sigma) (1:10,000 dilution). Protein yield was ranging between 10⁻¹⁵% of loaded protein sediment material as determined by BCL method (FIG. 1B).

Proteins were stored at neutral pH in PBS at −80° C. until use. The preparations were tested for ADP-ribosylation using the NAD-agmatine assay. Briefly, the ADP-ribosyltransferase activity of the fusion proteins was determined by assessing incorporation of [U-¹⁴C] adenine. After 60 min of incubation, eluates retrieved from AG1-X4 columns containing [U-¹⁴C] adenine-labeled ADP-ribosyl-agmatine were collected for determinations of radioactivity. The cpm activity of the fusion proteins was calculated from a standard curve generated by dilutions of intact CT (List Biologicals Laboratories, Cambell, CA) (FIG. 1E).

FIG. 1C illustrates an alignment from the CTA1 in CTA1-DD vs CTA1-CD103 constructs and FIG. 1D is a Western blot analysis of the CTA1-II-DD (1), CTA1(R7K)-II-DD (2), CTA1-II-CD103 (3) and CTA(R7K)-II-CD103 (4) molecules.

### Example 2

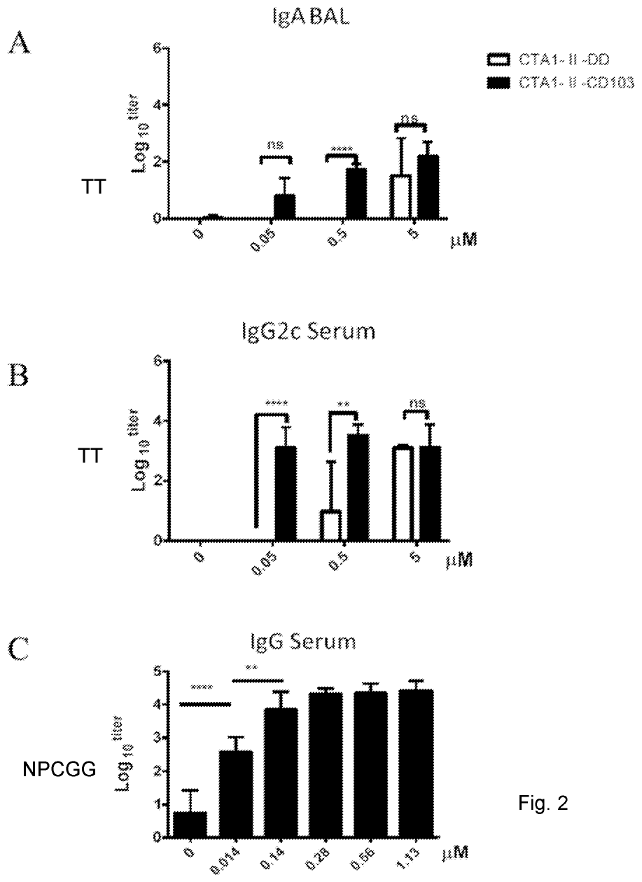

The adjuvant capacity of the CD103-targeted fusion protein for induction of antibody responses was compared to that of the DD construct. To this end C57Bl/6 mice (female mice aged 6-12 weeks) were immunized three times intranasally (i.n.) (20 ml) with tetanus toxoid (TT) (5 mg) or NP-hapten conjugated to chicken gamma globulin (NP-CGG) (5 mg) and admixed with different doses (mM) of CTA1-CD103 adjuvant constructions (Example 1) or CTA1-DD adjuvant constructs (Agren et al., J Immunol 1997 158(8): 3936-3946). Because immune protection at mucosal membranes is mediated by local production of secretory IgA (SIgA) we evaluated bronchial lavage (BAL) for presence of anti-TT or NP-specific SIgA log₁₀ titers at 7 days after the last immunization using ELISA. The CD103-targeted adjuvant was significantly more effective at promoting specific SIgA responses in BAL (FIG. 2A). Apart from local production of SIgA, protection against viral infections, such as influenza, is mediated by serum IgG antibodies and, in particular, IgG2a (Balb/c mice)/IgG2c (C57Bl/6 mice) antibodies. The serum antibody response to i.n. immunizations was clearly much stronger after CTA1-CD103 adjuvant immunizations as compared to immunizations using the CTA1-DD adjuvant as assessed by ELISA (FIG. 2B). In fact, while the IgG2c serum response was strong in the CTA1-CD103 group it was undetectable in the CTA1-DD group at a dose of 0.05 mM adjuvant and comparable titers were only reached at a dose of 5 mM, i.e., a 100-fold higher dose of CTA1-DD. A similar requirement for a 100-fold increased dose of CTA1-DD also applied to SIgA responses in BAL (FIG. 2C). Thus, CTA1-CD103 was exceptionally much stronger than CTA1-DD as an i.n. adjuvant and, indeed, the dose required for detection of a serum IgG responses following i.n. immunizations was 0.014 mM or less, which is >350-fold lower than the standard CTA1-DD adjuvant dose (5 mM) used for i.n. immunizations. This is a representative experiment of three independent experiments with a similar result. Each group hosts five mice and values are mean log₁₀-titers±SD Statistical significance was calculated with ANOVA with Dunnetts post-test; p<0.001***.

### Example 3

The capacity of the CTA1-CD103 adjuvant system to stimulate CD4 T cell responses was analyzed in C57Bl/6 mice (6-12 week old female mice) following i.n. immunizations. Both CTA1-CD103 and CTA1-DD were used at a saturating dose of 5 mM and a single immunization to mice that had been adoptively transferred with CFSE-labeled OTII CD4 T cells (10⁶ cells), specifically recognizing the p323 peptide from ovalbumin, which was also incorporated into the adjuvant constructs; CTA1-p323-CD103 and CTA1-p323-DD, respectively. This way we could assess the immune priming efficacy of CD4 T cells with this MHC class II-restricted peptide. Following i.n. immunization we evaluated the CD4 T cell response (OTII cells) after 4 days in the draining mediastinal lymph node (mLN) and compared equimolar dose of the adjuvants. For control purposes one group of mice were given an equimolar dose of ovalbumin i.n. and another group of mice were unimmunized. The CD4 T cell (OTII cells) proliferation was detected by monitoring cell division as assessed by the dilution of the CFSE-signal from each cell by FACS. The response was also evaluated by ELISPOT for the differentiation of the expanding CD4 T cells (OTII cells) into different functional subgroups, such as the Th1 and Th17 subsets, producing IFNγ and IL-17, respectively. To assess if the CTA1-enzyme was needed for the adjuvant action we engineered a fusion protein, CTA1R9K-p323-CD103, which lacked ADP-ribosylating enzymatic activity due to the R9K mutation in the CTA1-moiety (assessed by the agmatine assay). The effect of this fusion protein was compared to that of the intact CTA1-p323-CD103 in equimolar doses.

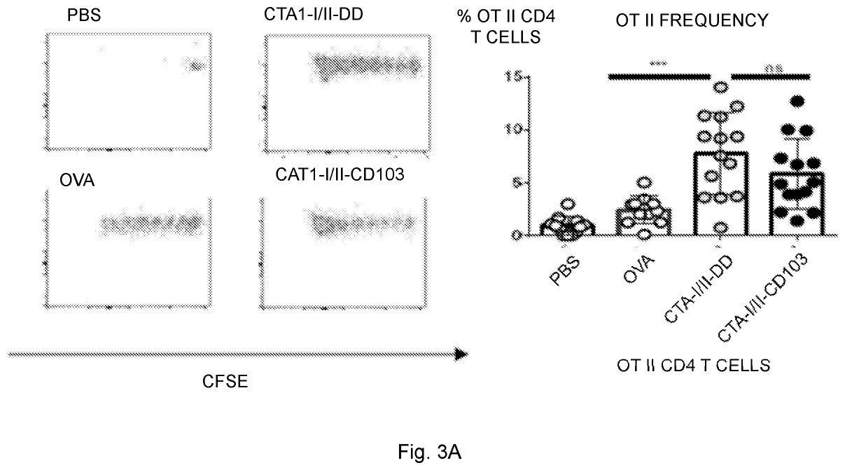

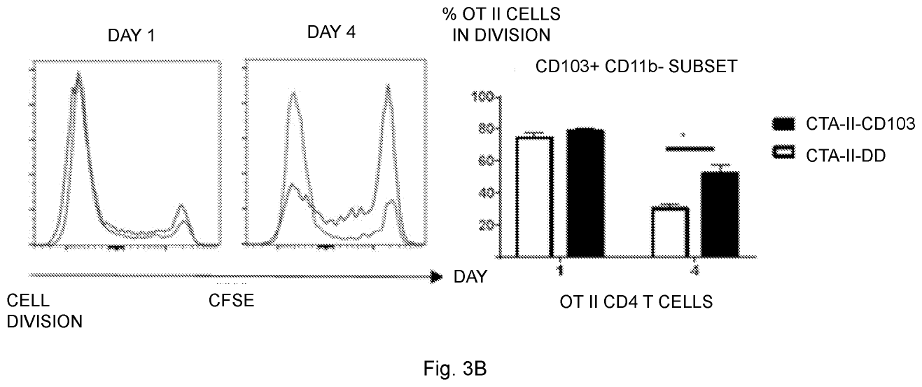

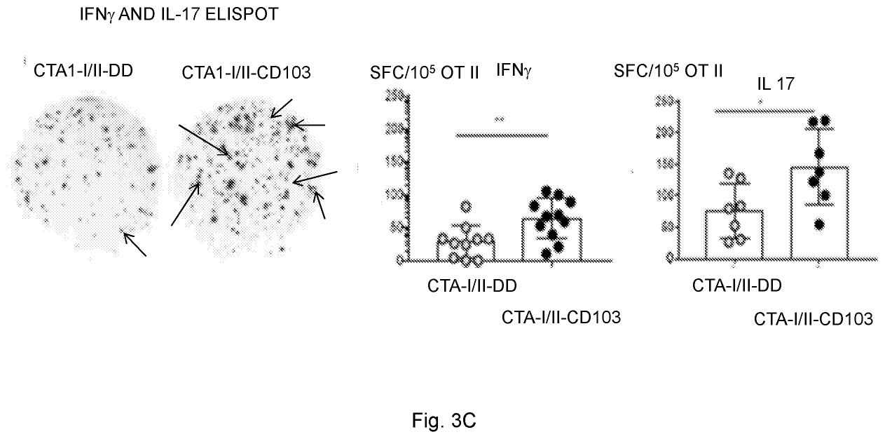

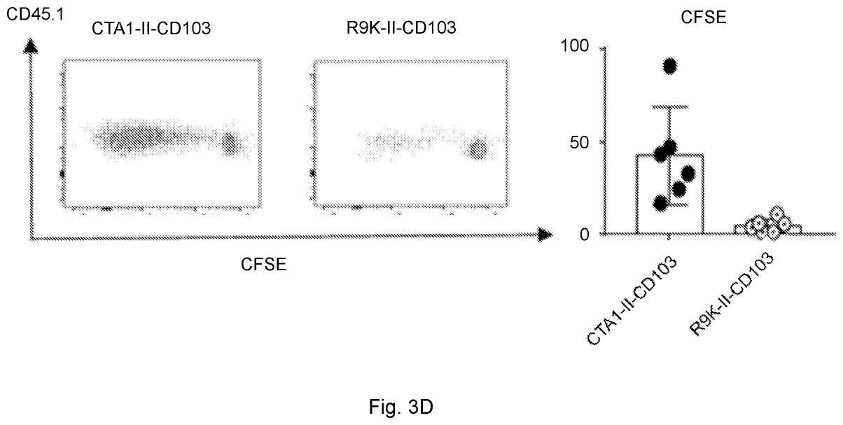

Both CTA1-p323-CD103 and CTA1-p323-DD in saturating 5 mM doses i.n. were effective at stimulating CD4 T cell activation and cell division as assessed by FACS and specific antibodies (FIG. 3A). However, CTA1-p323-CD103 was significantly more effective compared to CTA1-p323-DD at stimulating OTII proliferation, especially if OTII cells were given to the i.n. primed mice after 4 days (FIG. 3B). Also, differentiation to cytokine production was better with the CTA1-p323-CD103 construct compared to the CTA1-p323-DD fusion protein as determined by the ability to produce IFNγ (Th1) or IL-17 (Th17) by OTII CD4 T cells following i.n. immunization (FIG. 3C). In particular, CTA1-p323-CD103 was effective at promoting Th17 cells. The enzyme-activity of CTA1 was absolutely crucial for the adjuvant effect of CTA1-p323-CD103 constructs, as determined by FACS and using specific antibodies (FIG. 3D). These are a representative experiments of three independent experiments in each category with similar results. Each group hosts five mice and T cell responses were analyzed by FACS and values are mean±SD assessed after 4 days following immunization or adoptive transfer of OTII cells. ELISPOT values are given as spot forming cells (SFC/10⁶ cells). Statistical significance was calculated with ANOVA with Dunnetts post-test; p<0.001***.

### Example 4

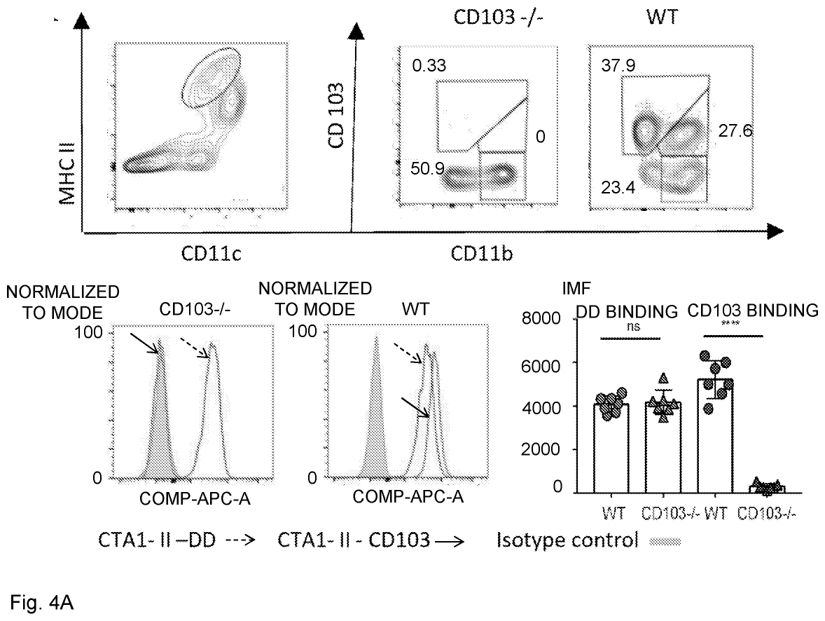

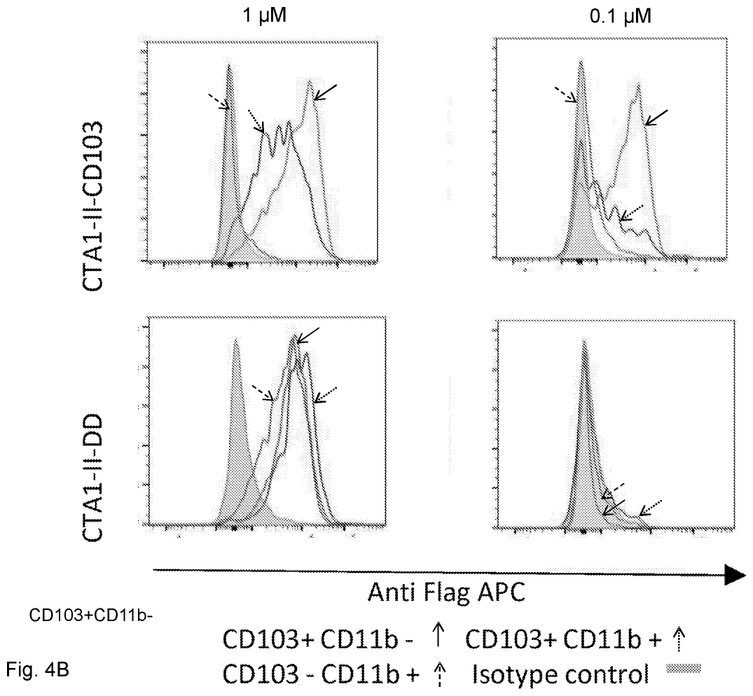

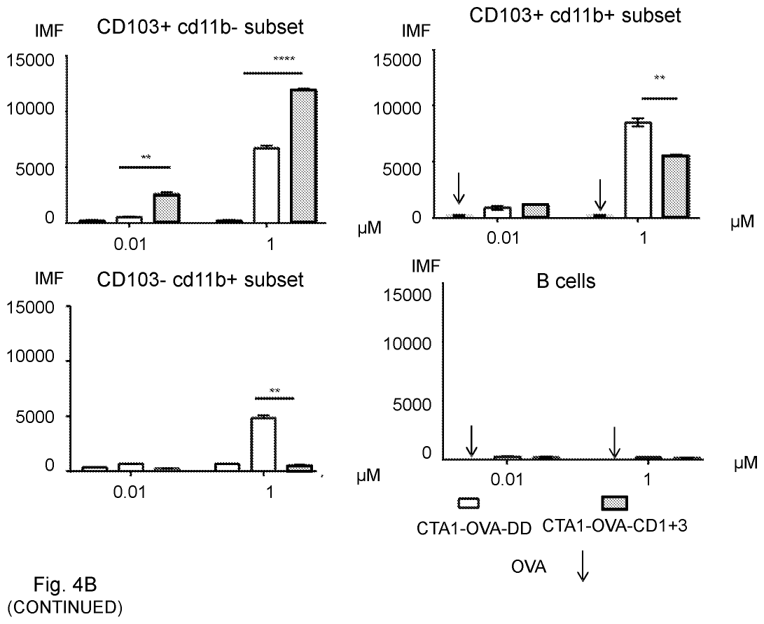

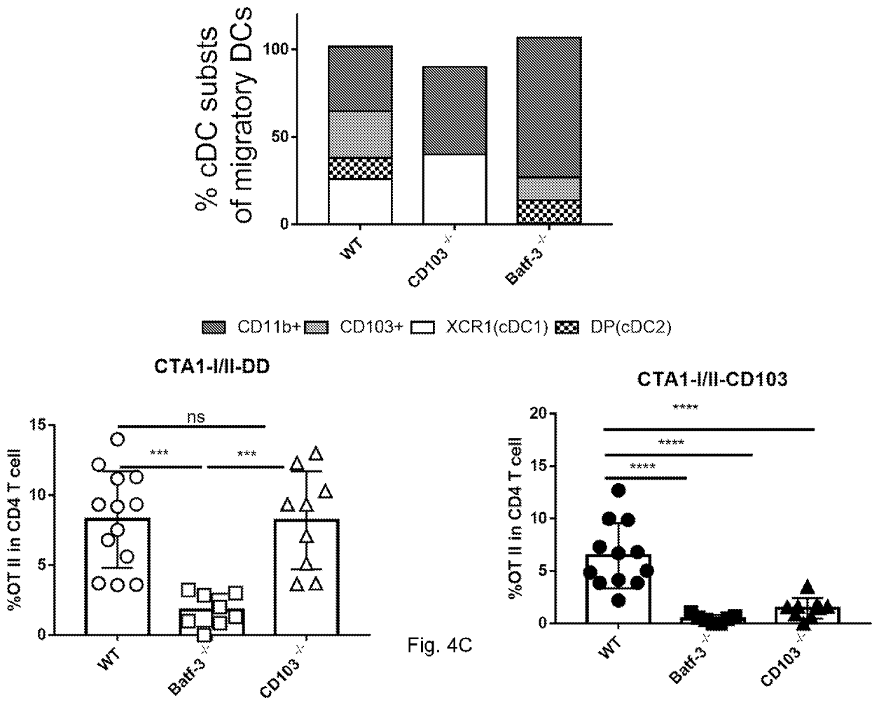

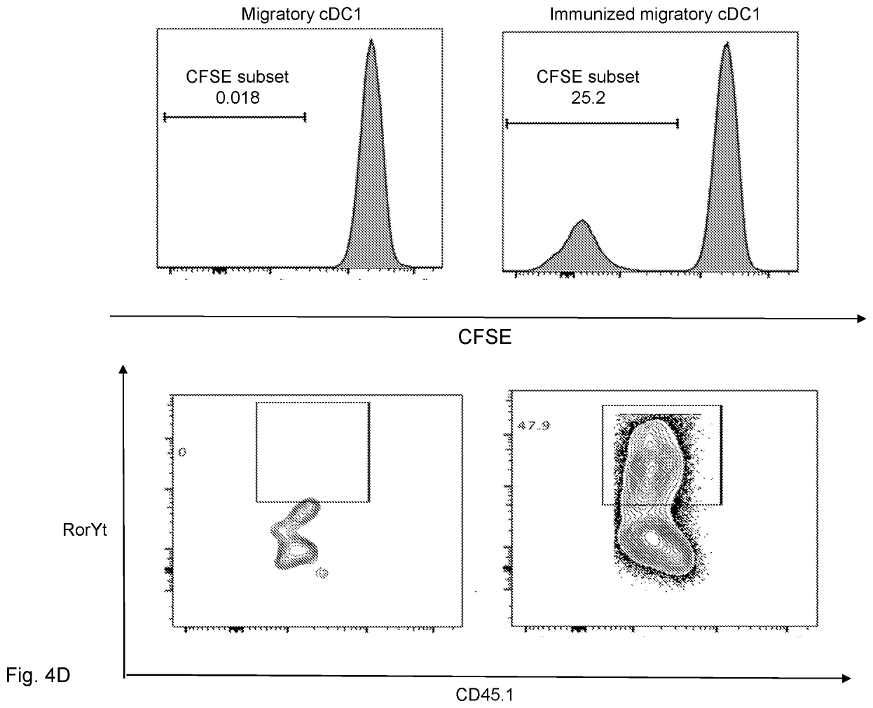

The CTA1-CD103 adjuvant targets dendritic cells (DC) and, in particular, the cDC1 subset, which is critically mediating the effect of the adjuvant function in vivo. To demonstrate which DC subset that specifically was targeted by the CTA1-CD103 adjuvant constructs, migratory DCs (MHC^{high}/CD11c^{high}) cells from the draining mediastinal lymph node (mLN) were isolated and investigated for binding the adjuvant by FACS using specific antibodies. Equimolar doses (1 mM) of CTA1-DD were used for comparisons. CTA1-CD103 adjuvant, exclusively bound to CD103-expressing DC subsets and when mice with a genetic deficiency for CD103 (CD103^{-/-} mice) were used the CTA1-CD103 adjuvant failed completely to bind to DCs (FIG. 4A). The CTA1-CD103 construct specifically targeted CD103⁺ DCs and, in particular cDC1 cells (CD103⁺, CD11b⁻), albeit some adjuvant also bound to cDC2 cells (CD103⁺, CD11b⁺). However, in lower concentrations (0.1 mM) the CTA1-CD103 adjuvant bound only cDC1 cells (FIG. 4B). This was in contrast to CTA1-DD which bound to all three DC populations, including the single positive cDC2 subset (CD103⁻, CD11b⁺) (FIG. 4B). Hence, CTA1-DD had a broader binding repertoire to DCs compared to the CTA1-CD103 adjuvant. This was confirmed in vivo when mice with distinct deficiencies were used for immunizations and for comparing the effect of the CTA1-CD103 adjuvant with that of the CTA1-DD construct. Mice, completely lacking CD103-expressing DCs (CD103^{-/-}) or with no cDC1 cells (Batf3^{-/-}), did not respond with CD4 T cell activation/proliferation (OTII cells and CFSE-dilution) as assessed by FACS to a single i.n. immunization with CTA1-CD103 (5 mM), while responses to an equimolar dose of CTA1-DD were intact (CD103^{-/-} mice) or could be detected (Batf3^{-/-}) (FIG. 4C). Interestingly, naive CD4 T cells (OTII cells) were responding with strong proliferation (CFSE-dilution) and differentiation towards a Th17 phenotype (rorgt⁺) upon co-culture with freshly isolated cDC1 cells after 24 h following an i.n. administration of CTA1-p323-CD103 adjuvant (Stem cell kit) (FIG. 4D). Importantly, previous data do not support that cDC1 cells can promote Th17 differentiation and, so, the finding is unexpected and novel (FIG. 4D). The migratory cDC1 cells were isolated from the mLN after an i.n. immunization with CTA1-p323-CD103 and co-cultures of DCs (10.000 DCs) and naive CD4 T cells (100.000 OTII cells in each well) were tested. This resulted in Th17 differentiation (rorgt+) of the CD4 T cells and IL-17 production in vitro as assessed by FACS and ELISA (FIG. 4D). These are a representative experiments of three independent experiments in each category giving similar results. In vitro cultures were performed in triplicates and values are given as mean±SD. For in vivo experiments each group hosts five mice and T cell (OTII cells) responses were analyzed by FACS and values are mean±SD assessed after 4 days following immunization. Statistical significance was calculated using ANOVA with Dunnetts post-test; p<0.001***.

### Example 5

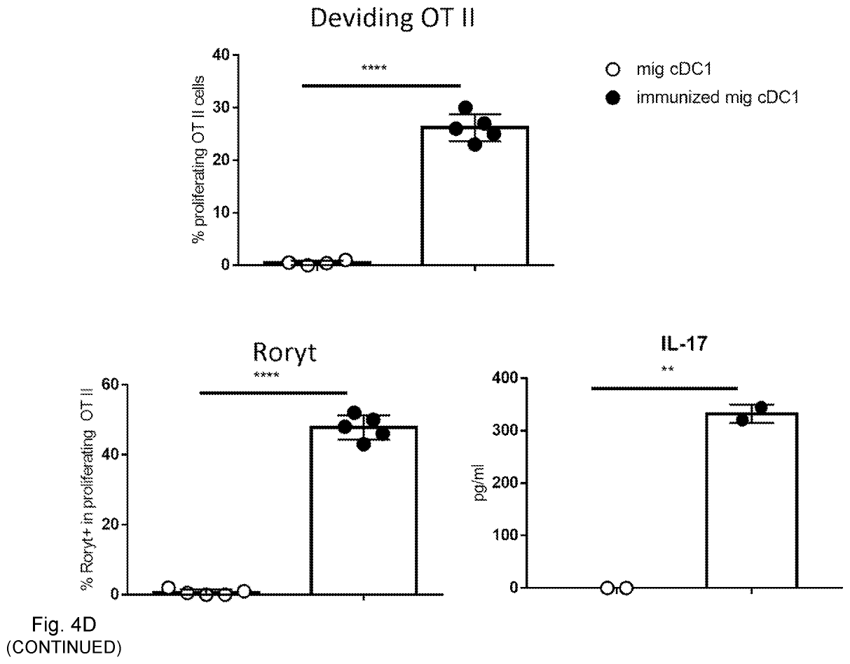

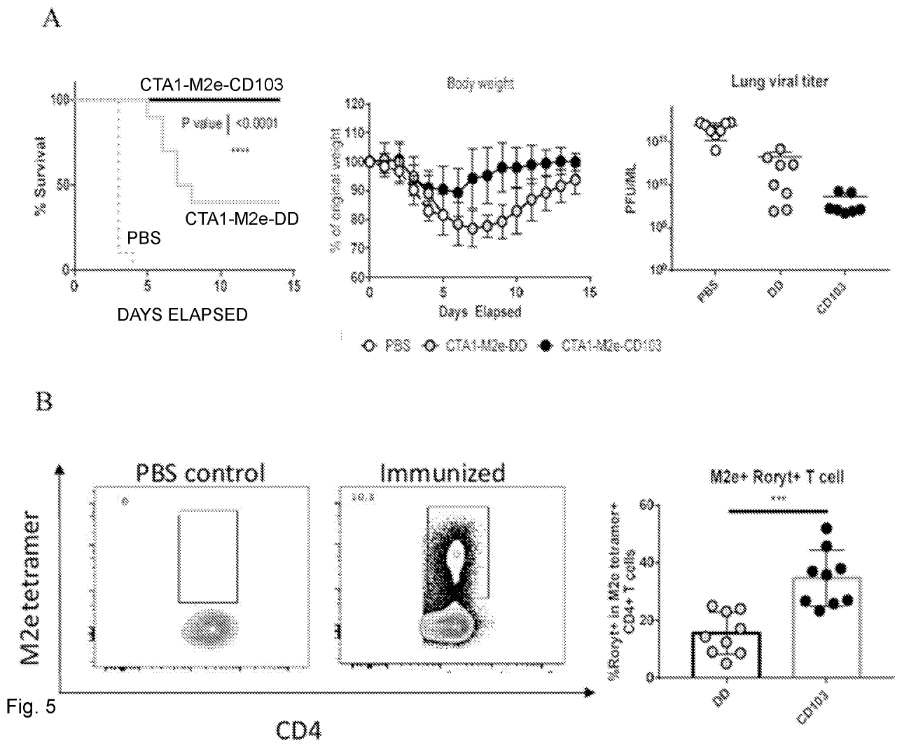

The CTA1-CD103 adjuvant is significantly more effective compared to the CTA1-DD adjuvant at stimulating protective immunity against a live virus infection. The ability to stimulate protective immunity was tested in the influenza virus mouse model. Balb/c mice were immunized with CTA1-3M2e-CD103 or CTA1-3M2e-DD (5 mM), hosting a vaccine epitope (M2e) specific for influenza A viruses, to assess the capacity to stimulate immune protection against a live challenge infection with influenza virus. The M2e peptide is a CD4 T cell restricted epitope. Following three i.n. immunizations with saturating and equimolar doses (5 mM) of the fusion proteins the mice were given a live challenge infection with 4×LD₅₀ of the PR8 heterosubtypic virus 3 weeks after the last immunization. Mice were then monitored for the weight loss and survival for 2 weeks after challenge. All mice that were immunized with CTA1-3M2e-CD103 survived the challenge infection, whereas more than 50% of the mice succumbed in the CTA1-3M2e-DD immunized group (FIG. 5A). Mice that were immunized with CTA1-3M2e-CD103 had significantly reduced viral titers in the lung (FIG. 5A). The CTA1-3M2e-CD103 fusion protein was much more effective at stimulating M2e-specific CD4 T cells as identified by an M2e-specific tetramer and FACS (FIG. 5B). Lung resident M2e-specific memory CD4 T cells were much more frequent in mice immunized with CTA1-3M2e-CD103 as compared to the CTA1-3M2e-DD construct. (FIG. 5B). Most of the M2e-specific CD4 T cells in the lung were of the Th17 type (rorgt+). Thus, the CTA1-CD103 adjuvant system was superior at stimulating full protection against influenza virus infection, a mechanism that correlated to an enhanced M2e-specific Th17 response. These are a representative experiments of three independent experiments in each category giving similar results. Each group hosted 10 mice and M2e-specific CD4 T cells were analyzed by FACS and values are means±SD assessed after 10 days following the challenge infection. Statistical significance was calculated using ANOVA with Dunnetts post-test; p<0.001***.

### Example 6

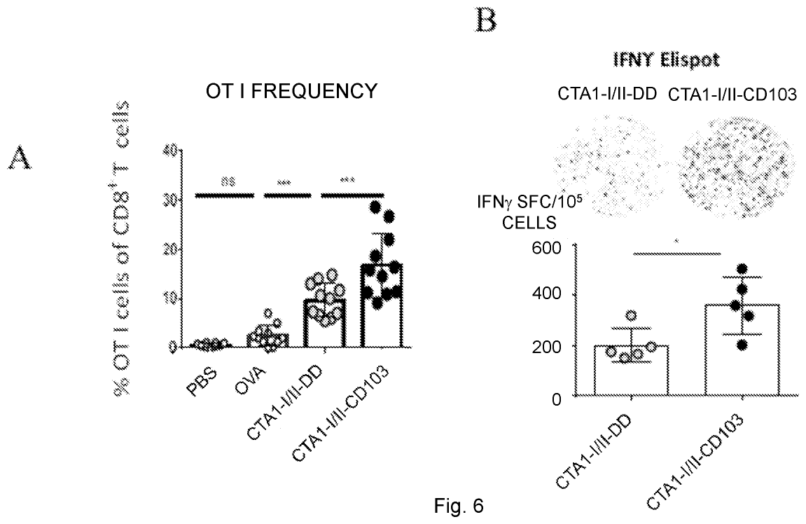

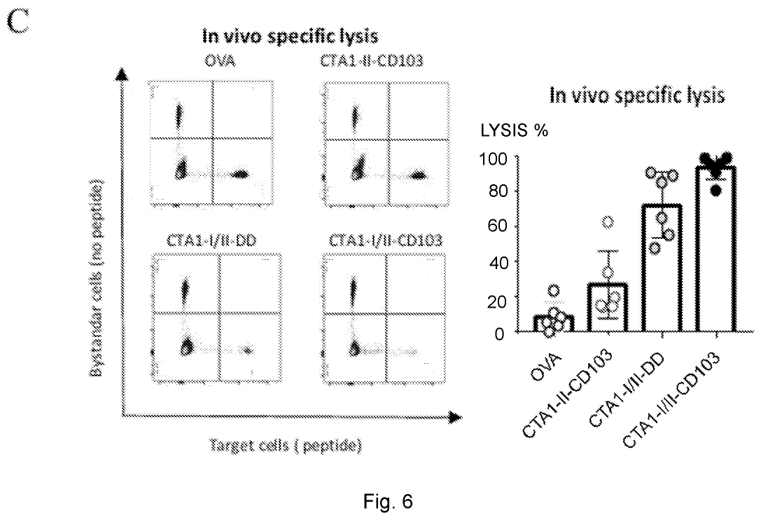

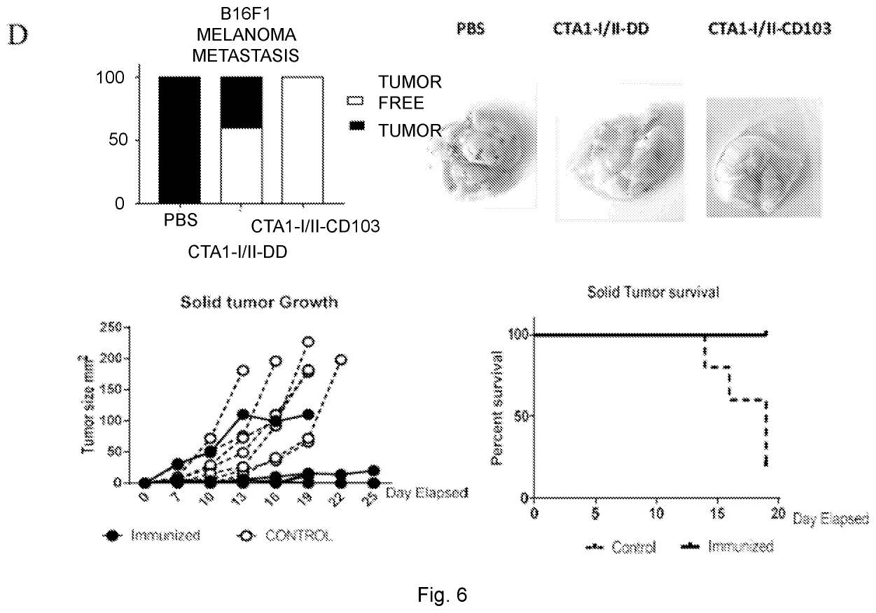

The CD103-DC-targeted CTA1-CD103 adjuvant effectively stimulates cytotoxic CD8 T cells and prevents cancer cell growth and metastasis. The MHC class I-restricted epitope from ovalbumin SIINFEKL was used in the fusion protein, CTA1-SIINFEKL-CD103, to determine whether the adjuvant system could stimulate CD8 T cell-restricted cytotoxicity in the mouse model. The effect was compared to that of the CTA1-SIINFEKL-DD following i.n. immunizations with equimolar doses (5 mM) of the adjuvants. First we established that cross-presentation of MHC class I-restricted peptides, such as the SIINFEKL, could be promoted by the targeted adjuvant molecules. This was critical because the CD103⁺ cDC1 cells are known for this function (FIG. 6A). The CTA1-SIINFEKL-CD103 was superior to the DD-construct for its ability to promote cross-presentation and induction of CD8 T cells and cytotoxicity following i.n. immunizations (FIGS. 6A-6C). The effect on promoting cytotoxicity and prevention of tumor growth as well as metastasis was evident using the ovalbumin expressing melanoma B16F1 cell model after either prophylactic or therapeutic i.n. immunizations with 5 mM doses of the fusion proteins (FIG. 6D). Both a model for metastasis and one for solid tumor growth were used and it was found that transfer of 200.000 B16F1 cells into recipient mice were sufficient to establish metastasis or solid tumors. The solid tumor was allowed to establish over 2 weeks and the therapeutic vaccination was given when the tumor had reached the size of a rice grain. Cytotoxicity was assessed in SIINFEKL-specific OTI cells following adoptive transfer into C57Bl/6 mice, which had been prophylactically i.n. immunized three times with 10 days inbetween. Cytotoxicity was assessed 5-7 days after the administration of the labelled B16F1 melanoma cells using FACS. These are a representative experiments of three independent experiments in each category giving similar results. Each group hosted 3-5 mice and OTI-specific CD8 T cells were analyzed by FACS and values are mean±SD. Statistical significance was calculated using ANOVA with Dunnetts post-test; p<0.001***.

### Example 7

Lipid-based nanoparticles have in recent years attracted increasing attention as pharmaceutical carriers. In particular, reports of them having inherent adjuvant properties combined with their ability to protect antigen from degradation make them suitable as vaccine vectors. However, the physicochemical profile of an ideal nanoparticle for vaccine delivery is still poorly defined. Here, we used an in vitro dendritic cell assay to assess the immunogenicity of a variety of liposome formulations as vaccine carriers and adjuvants. Using flow cytometry, we assessed liposome-assisted antigen presentation, as well as the expression of relevant co-stimulatory molecules on the cell surface. Cytokine secretion was further evaluated with an ELISA assay. We show that liposomes can successfully enhance antigen presentation and maturation of dendritic cells, as compared to vaccine fusion protein (CTA1-3Eα-DD) administered alone. In particular, the lipid phase-state of the membrane, was found to greatly influence the vaccine antigen processing by dendritic cells. As compared to their fluid phase counterparts, gel phase liposomes were more efficient at improving antigen presentation. They were also superior at upregulating the co-stimulatory molecules CD80 and CD86, as well as increasing the release of the cytokines IL-6 and IL-1β. Taken together, we demonstrate that gel phase liposomes, while non-immunogenic on their own, significantly enhance the antigen-presenting ability of dendritic cells and appear to be a promising way forward to improve vaccine immunogenicity.

### Materials and Methods

All chemicals were purchased from Sigma Aldrich, Sweden, and all lipids from Avanti polar lipids, U.S., unless otherwise stated. The lipids used were 1,2-dioleoyl-sn-glycero-3-phosphocholine (DOPC), 1,2-distearoyl-sn-glycero-3-phosphocholine (DSPC), 1,2-distearoyl-sn-glycero-3-phosphoethanolamine-N-[maleimide(polyethylene glycol)-2000] (DSPEPEGMCC). The fusion proteins were provided by MIVAC, Sweden.

### Particle Preparation

Liposomes were produced by the lipid film rehydration and extrusion method. Different solutions of lipids dissolved in a methanol/chloroform (1:1 v/v %) mixture were added to round-bottom flasks. The solvents were evaporated in a warm water bath under reduced pressure (fluid phase liposomes) or nitrogen flow (gel phase liposomes) for approximately 30 minutes followed by vacuum overnight.

To produce the fluid phase liposomes, a thin lipid film composed of DOPC and DSPEPEGMCC (99:1 mol %) was then rehydrated in NaAc saline (10 mM sodium acetate, 150 mM NaCl, pH=5.0) to an 8 mM lipid concentration. The suspension was then freeze-thawed 10 times using liquid nitrogen and a 50° C. water bath. The suspension was further diluted with NaAc saline to a lipid concentration of 4 mM and extruded 21 times through a 100 nm nucleopore track-etched polycarbonate membrane (Whatman, UK) using a mini extruder (Avanti Polar Lipids Inc, USA) and 1 bar pressure.

To produce gel phase liposomes, a thin lipid film composed of DSPC and DSPEPEGMCC (99:1 mol %) was rehydrated in NaAc saline to a lipid concentration of 8 mM. Rehydration took place in a 65° C. water bath for one minute, followed by at least 10 cycles of freeze-thawing and gentle vortexing. The liposome suspension was then extruded 21 times through a 100 nm nucleopore track-etched polycarbonate membranes at 70° C. using a mini extruder and 1 bar pressure.

The fusion protein was covalently bound to the liposomes using a thiol-maleimide reaction after converting primary amines to thiol groups using Traut's reagent (2-iminothiolane hydrochloride). Briefly, Traut's reagent (0.02 mg/ml in HBS: 10 mM HEPES, 150 nM NaCl, with 2 mM EDTA, pH=7.8) and fusion protein (1.6 mg/ml in 10 mM NaH₂ PO₄, 0.16 M NaCl, pH 7.4) were mixed to a volume ratio of 5:3 and incubated for 20 minutes at 4° C. Particles at a lipid concentration of 4 mM were added to freshly thiolated fusion protein at a volume ratio of 5:4 and incubated at room temperature for 1 h with gentle shaking. Unreacted fusion protein was removed using Amicon Ultra 100 kDa cut-off centrifugal filters (VWR, Sweden) as follows: 125 μl NaAc saline and approximately 290 μl liposome suspension was added to each filter and centrifuged (5 min or until approximately 250 μl solution remained, 8000×g, 10° C.) followed by a further dilution with 150 μl NaAc saline in each filter and another centrifugation (5 min or until approximately 250 μl solution remained, 8000×g, 10° C.). The suspension was recovered by inverting the filters and centrifuging (1 min, 8000×g, 10° C.). Lipid particle suspensions were stored at 4° C. until use.

### Total Protein Quantification

The fusion protein content was determined using the CBQCA Protein Quantitation Kit (Thermo Fisher Scientific Inc., Sweden), according to manufacturer's instructions. When relevant, the amount of encapsulated antigen was determined by measuring the protein content of particles to which no fusion protein had been covalently attached. In all cases, a 5 mM stock solution of the CBQCA reagent was used and 0.1% Triton X-100 was added to the reaction buffer. The reaction was performed in a 96-well plate and the fluorescence was measured using a FLUOstar OPTIMA microplate reader (BMG Labtech, Germany) (excitation: 440 nm, emission: 520 nm).

### Particle Size and Concentration Determination

The hydrodynamic diameter of the liposomes was determined by nanoparticle tracking analysis (NTA) using a NanoSight LM10 (Malvern, UK) equipped with a Hamamatsu C11440-50B/A11893-02 camera and a 488 nm laser. A measurement consisted of five 60-second videos. Analysis was performed using NTA software version 3.2 with camera level: 11 and detection threshold: 2.

The liposome concentration was determined using NTA by measuring three different dilutions, usually 10 000, 20 000 and 40 000×. Comparative measurements were done at concentrations within the range where there is good agreement between actual and measured concentrations, on the same day and using the same protocol, to keep relative variations small.

### Estimation of Number of Fusion Proteins Per Particle

The mean number of fusion proteins per liposome was calculated from the protein content values obtained via CBQCA and the particle concentration measured with NTA.

### Measurement of Zeta Potential Using Laser Doppler Electrophoresis

The particles' zeta potentials were measured in 2 mM HEPES buffer (buffer exchange was done using illustra Microspin S-200 HR columns, according to the manufacturer's instructions (Fisher Scientific, Sweden)), pH=7.4 at 25° C. using a Zetasizer Nano ZS (Malvern, UK) with a DTS1070 folded capillary cell. The viscosity of the dispersant was set to 0.8872 cP. The refractive index of the dispersant and the liposomes were set to 1.33 and 1.45, respectively and the Smoluchowski approximation was used.

### Time-Dependent Antigen Presentation Using Flow Cytometry

To assess how different vaccine formulations influence antigen processing ability of dendritic cells, an in vitro model comprising the mouse-derived dendritic cell line D1 together with the Eα/YAe system was used. When bound to the MHC II, the Eα peptide can be detected using the monoclonal YAe antibody, thus allowing quantification of functionally presented antigen. The D1 cell line, which is of splenic origin and derived from a female C57BL/6 mouse, was kindly provided by P. Ricciardi-Castagnoli (University of Milan-Bicocca, Milan, Italy). The cells were cultivated in Iscove's Modified Dulbecco's Medium (Biochrom, Germany), supplemented with 10% heat-inactivated fetal calf serum (Biochrom, Germany), 50 μM 2-mercaptoethanol, 1 mM L-glutamine (Biochrom, Germany) and 50 μg/ml Gentamycin and 30% NIH/3T3 supernatant at 37° C. and 5% CO₂. Cells were plated in 24-well plates and allowed to attach before activation with different formulations of fusion protein, at a final protein concentration of 0.2 μM, for different amounts of time.

Prior to the flow cytometry analysis, the D1 cells were mechanically harvested, washed and stained with 7AAD, CD11c-PE, MHC II-FITC and YAe-biotin recognizing E 52-68 peptide bound to I-Ab molecules to assess the amount of functional antigen presentation. After incubation for 30 minutes at 4° C. followed by washing, a secondary staining with Streptavidin-Brilliant Violet 421 was performed using the same experimental parameters as for antibody incubation. All antibodies were from eBiosciences, USA. Cells were washed again and analyzed using a BD-FACS LSR II (BD Bioscience, USA). Data analysis was performed using FlowJo (TreeStar, USA).

### Quantification of Antigen Presentation and Co-Stimulatory Molecules at 24 Hours Using Flow Cytometry

D1 cells were cultured as previously described and activated with different lipid particle formulations: DOPC and DSPC liposomes containing 1% DSPEPEGMCC coupled to fusion protein (DOPC-PEG-FP and DSPC-PEG-FP), DOPC and DSPC liposomes containing 1% DSPEPEGMCC co-administered with free fusion protein (DOPC-PEG+free FP and DSPC-PEG+free FP), and DOPC and DSPC liposomes containing 1% DSPEPEGMCC given without fusion protein (DOPC-PEG and DSPC-PEG). After 24 hours incubation, the supernatants were taken aside and stored at −20° C. until cytokines enzyme-linked immunosorbent assay (ELISA) was performed for cytokine quantification.

D1 cells were mechanically harvested, washed and stained with 7AAD, CD11c-BV421, MHC II-PE, CD80-A647, CD86-BV605, and YAe-biotin for 30 min at 4° C. Cells were washed and a secondary staining with Streptavidin-APC/Cy7 was performed. After washing cells were analyzed using a BD LSR Fortessa (BD Bioscience, U.S.). Data analysis was performed using FlowJo (TreeStar, U.S.).

### Quantification of Released Cytokines Using Enzyme-Linked Immunosorbent Assay

Quantification of cytokines in the supernatants was done using DuoSet ELISA kits (R&D systems, USA). Briefly, 96-well microplates (MaxiSorp, Nunc, Denmark) were incubated with 50 μl/well capture antibody in a humidity chamber overnight at room temperature. After washing 3 times with PBS with 0.05% Tween, 50 μl/well recombinant standard solution or 25 μl/well supernatant in duplicates were added and the plates were incubated (2 hours, in humidity chamber, room temperature). The plates were washed and 50 μl capture antibody was added and incubated (2 hours, in humidity chamber, room temperature). The plates were washed and 50 μl/well streptavidin-horseradish-peroxidase was incubated for 20 minutes (in humidity chamber, room temperature). The plates were washed and 50 μl/well substrate solution was added. Following 20 minutes incubation in humidity chamber at room temperature in the dark, 25 μl/well stop solution was added and adsorption was read at 450 nm.

### Statistics

Quantitative values were presented as mean±standard error of the mean, unless otherwise stated. Statistical comparisons for change in cumulative YAe and MHC II intensities, costimulatory receptors and amount of cytokine release were performed using one-way analysis of variance and Tukey post-hoc test. p<0.05 was considered statistically significant.

### Results

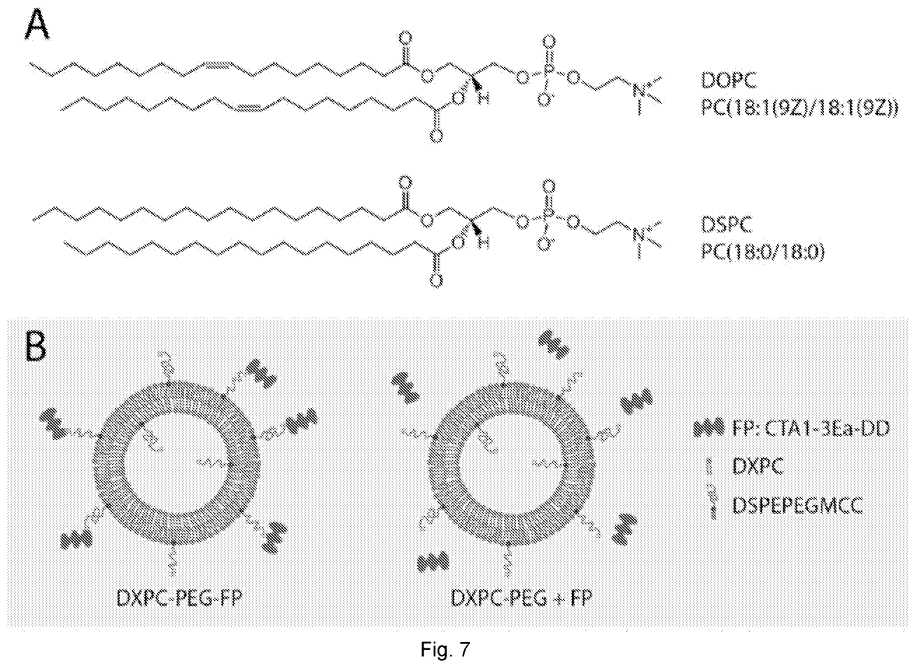

To select which physicochemical properties of the vaccine vectors deserved a more in-depth investigation, a preliminary screening using 6 different compositions was performed. This investigation addressed the influence of lipid composition, poly(ethylene glycol) (PEG) content, protein load and the size/shape of the nanoparticles, and showed that it was only the liposome formulation containing gel phase lipids that proved promising in the context of antigen presentation by DCs. The fact that lipodisks with the same phase-state as the DSPC-based liposomes have no effect suggest, however, that other factors such as size, shape, or amount of protein/particle may also play a role. To specifically study the effect of the phase state, we focus here on liposome formulations based on either 1,2-dioleoyl-sn-glycero-3-phosphocholine (DOPC) or 1,2-distearoyl-sn-glycero-3-phosphocholine (DSPC). Both of these phospholipids have two fatty acids of equal length (18 carbons), however, due to the presence of one unsaturation in each acyl chain, the DOPC lipids have a phase transition temperature of −17° C., while for DSPC, the fully saturated variant, it is 55° C. (FIG. 7A). As a result, DOPC-based membranes will be in a fluid phase-state when at physiological temperature, while the DSPC-based liposomes will be in the gel phase-state. Here, the CTA1-3Eα-DD fusion proteins (herein referred to as FP) were either coupled to the liposomes using DSPE-anchored PEG(2000)-spacers, or simply admixed with the liposomes (FIG. 7B).

### Particle Characterization

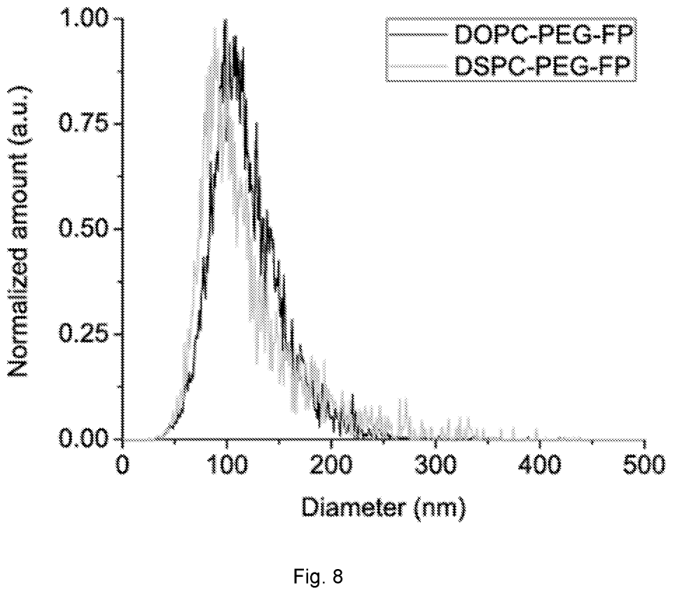

The vaccine formulations were characterized with respect to FP content, size and zeta potential (Table 3). The amount of FP coupled per particle was estimated from the total protein concentration (see Materials and methods) and from the total particle concentration estimated using nanoparticle tracking analysis (NTA). As shown in Table 3 and FIG. 8, the hydrodynamic diameter, full width at half maximum and zeta potential of the liposomes were independent of their composition. The protein load differed somewhat from one batch to the other, but within each individual cell experiment, the number of proteins/particle was comparable. The net charge of the zeta potential is consistent with both liposome types which mainly contain the zwitterionic phosphocholine (PC) lipids together with 1% lipids (DSPEPEGMCC), which are negatively charged at physiological pH and with the fact that the fusion protein is negatively charged at physiological pH.

TABLE 3
Summary of the physiochemical characteristics
of the different liposome carriers

| | DOPC-PEG-FP | DSPC-PEG-FP |
|---|---|---|
| # Proteins/particle (mean ± SD) | 117 ± 29 174 ± 100 | 174 ± 100 |
| Diameter (mean, nm) | 120 | 120 |
| Zeta potential (mV, ±SD) | −14.0 ± 0.6 | −14.3 ± 1.0 |
| Phase | fluid | gel |

### Gel Phase DSPC-Based Liposomes Increase Antigen Presentation on Dendritic Cells

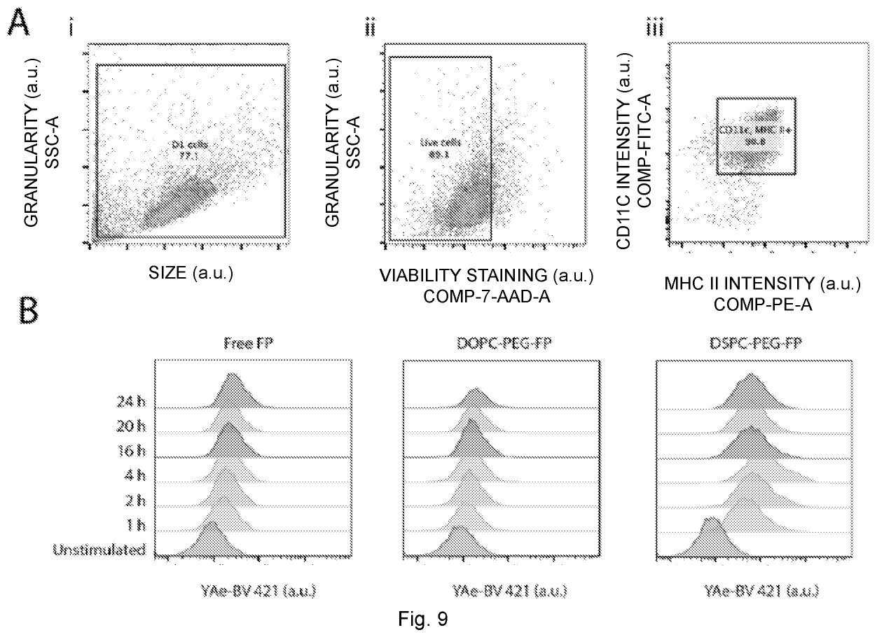

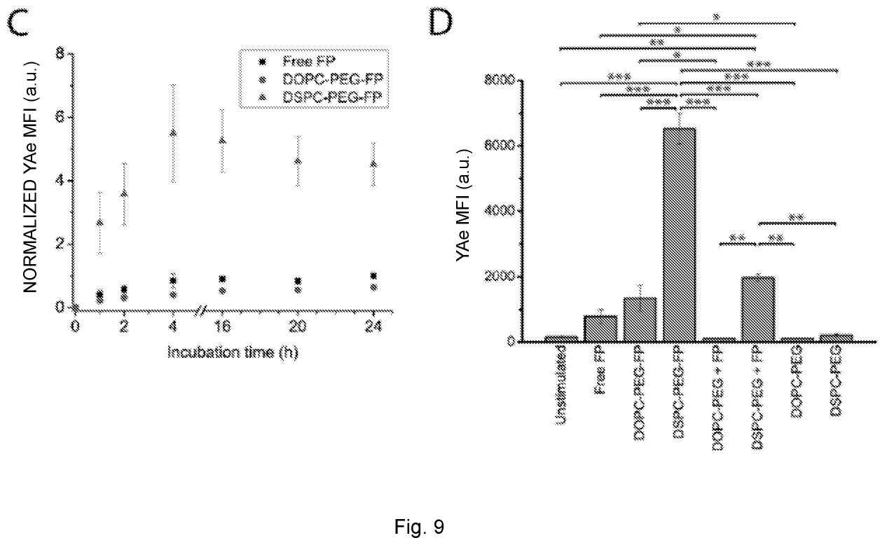

The ability to process vaccine antigen was analyzed for the different vaccine formulations using our in vitro model with D127 cells and the Eα/YAe mAb system. The level of YAe mAb binding reflected the Ea-MHC II complex expression on the D1 cells and was detected by flow cytometry. The assay was performed at a fixed fusion protein concentration (0.2 μM) and different incubation times ranging from 0 to 24 h. FIG. 9Ai-iii visualizes the gating procedure used for the flow cytometry analysis. We found a clear shift of the whole DC population with increased peptide presentation for the DSPC-PEG-FP formulation but not for the DOPC-PEG-FP as compared to soluble FP used alone (FIG. 9B). To evaluate antigen presentation over time, we plotted the median fluorescence intensity (MFI) of YAe against incubation time. The main increase in antigen presentation occurred within the first few hours and was followed by a plateau at later time points. This pattern was most pronounced for DSPC-PEG-FP (FIG. 9C), and was consistent across different datasets and with different batches of DSPC-PEG-FP. To provide a quantitative estimate of the peptide expression over the whole time period, we calculated a cumulative value for the signal induced by the various liposome formulation compared to free soluble FP for each individual dataset. The DSPC-PEG-FP value was a factor of 3.7±0.6 larger than that of free soluble FP, which further confirms that DSPC-PEG-FP induced a statistically significant increase in peptide presentation. On the other hand, the DOPC-PEG-FP formulation did not show a significant increase over the whole time span (factor 0.8±0.06).

To assess whether coupling of soluble FP to the nanoparticles was necessary to achieve a higher peptide expression level on the DC surface, we compared the signal (after 24 hours) induced by admixed DOPC-PEG+FP or DSPC-PEG+FP with the one generated by liposomes with coupled FP. Administration of FP together with DSPC-PEG, but not DOPC-PEG, liposomes resulted in increased peptide presentation, (albeit to a lesser extent than when the FP was coupled to the nanoparticle; ˜factor 2.5 vs. 8.5, FIG. 9D). This observation demonstrates that coupling of the FP to DSPC-PEG was beneficial, but that some augmenting effects on peptide presentation were also seen with DSPC-PEG+FP. As expected, control liposomes without FP did not result in any detectable binding of the YAe mAb (FIG. 9D).

### Gel Phase DSPC-Based Liposome Carriers Increase Peptide Loading Efficiency

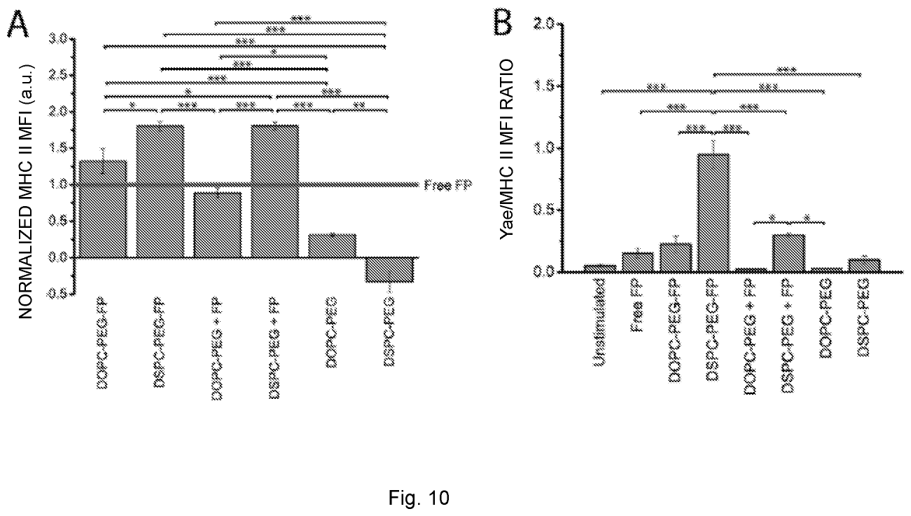

An antibody directed at the MHC II molecule was used to assess whether the increase in peptide presentation was accompanied by a corresponding increase in the level of MHC II molecules that were displayed on the cell surface. We found that after 24 hours the DSPC-PEG-FP and DSPC-PEG+FP formulations, but not DOPC-PEG vectors, gave an increased peptide expression, 1.8 fold compared to that seen with the FP alone (FIG. 10A). The MFI ratio of the peptide to MHC II molecule expression revealed that DSPC-PEG-FP appeared to increase peptide/MHC II occupancy by a factor of 6 as compared to that recorded for soluble FP alone (FIG. 10B). The increase was maintained over time, suggesting that the DSPC-PEG-FP affected the peptide loading efficiency of MHC II in the DC. However, as the soluble FP administered admixed with DSPC-PEG (DSPC-PEG+FP) resulted in only a marginal increase in improved peptide loading efficiency, it can be concluded that this feature was restricted to the DSPC-PEG-FP formulation, (FIG. 10B) indicating that the FP coupling to the nanocarrier is essential to the improved peptide loading efficiency.

### Gel Phase DSPC-Based Liposomes Also Enhance DC Co-Stimulation

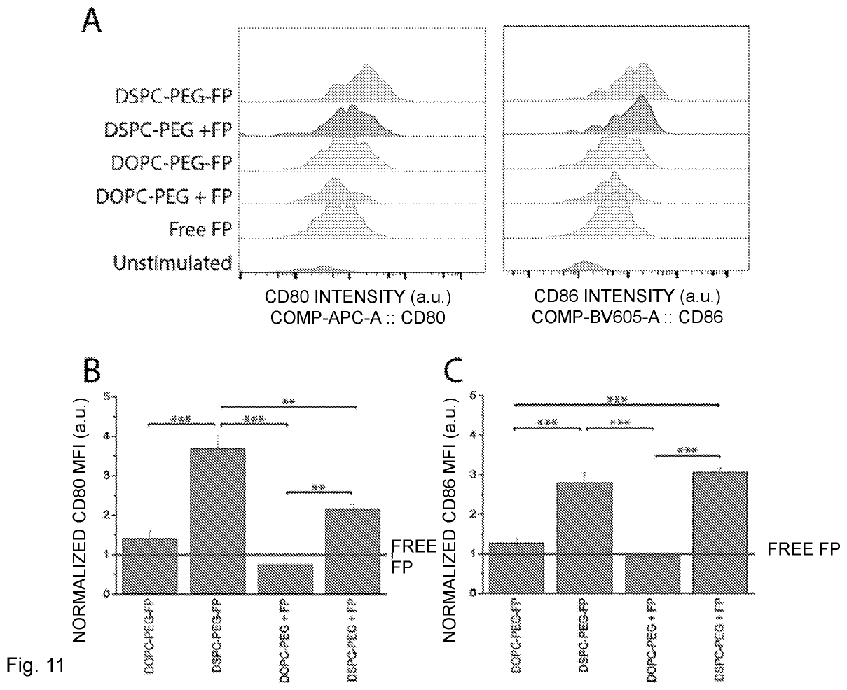

In addition to peptide presentation, DCs provide co-stimulatory signals to effectively prime T cells in the draining lymph node. These co-stimulatory signals are expressed at the DC cell surface and/or are secreted as soluble factors, i.e. cytokines. Among the most important cell surface co-stimulatory molecules known, are CD80 and CD86; their expression will greatly influence the ability of DCs to activate naïve T cells. Following culture for 24 h in the presence of the vaccine liposome formulations or soluble FP alone, we determined the expression of CD80 and CD86, respectively, using specific labeled mAbs and flow cytometry. Here, an additional gating step was introduced to select YAe+ cells. We found that the expression of co-stimulatory molecules followed the overall tendencies as we have seen for peptide presentation. Thus, CD80 and CD86 expression was significantly increased by DSPC-PEG-FP, by a factor 3.7 and 2.8, respectively, as compared to the free soluble FP, whereas DOPC-PEG-FP had only a marginal effect (factor 1.4 and 1.3 for CD80 and CD86, respectively). These results show that formulations with DSPC-based liposomes have a stronger stimulatory effect on co-stimulation than DOPC-based formulations (FIG. 11A). Interestingly, upregulation of CD86 expression appeared to increase irrespective of whether the FP was coupled to the liposome or admixed (FIG. 11C). This was not found with CD80 expression, where coupled DSPC-PEG-FP stimulated significantly higher CD80-expression than DSPC-PEG+FP (factor 3.7 and 2.2 compared to FP, respectively), while the latter was still slightly better than all DOPC-PEG variants (FIG. 11B). Very similar trends were observed when analyzing the whole population, and no increased expression of CD80 or CD86 was observed for cells treated with liposomes without fusion protein, indicating that the DSPC-PEG liposomes administered alone were poorly immunogenic.

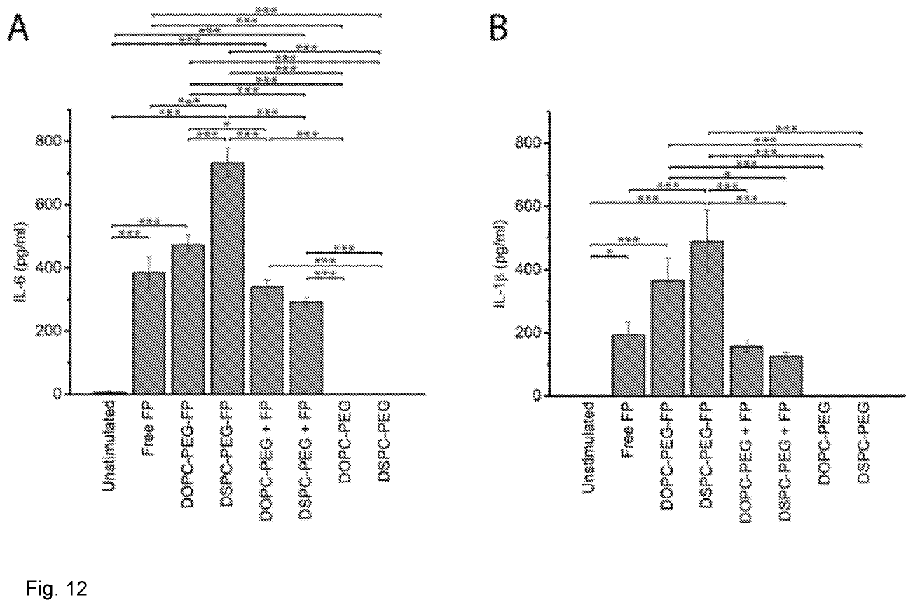

Next we analyzed whether the nanoparticles could affect production of relevant cytokines that could help in promoting T cell priming. We found that production of IL-6 and IL-1p was increased in culture supernatants from D1 cells incubated with DSPC-PEG-FP as compared to cultures stimulated with FP alone or DOPC-PEG-FP (IL-6 only), whereas cultures stimulated with DSPC-PEG+FP displayed no significant increase in IL-6 or IL-1β compared to free FP (FIG. 12). We can therefore conclude that the coupling of the FP to the DSPC-PEG liposomes is paramount to their ability to induce cytokine production and that overall, DSPC-PEG-FP is the most effective vaccine vector formulation to induce co-stimulation in targeted DCs.

We have taken a reductionist approach to elucidate whether the nanoparticle formulation can influence the antigen processing and T cell priming ability of a fusion protein. It should be noted that the fusion protein itself is a highly immunogenic molecule that carries a powerful adjuvant molecule, CTA1, and promotes DC priming functions. Using the combined vector we were interested to find out whether the lipid composition of the nanoparticle had an augmenting effect on the DC priming ability. This was investigated using a dendritic cell line in vitro; detection of changes in antigen presentation and co-stimulatory molecules on the DCs were analyzed by flow cytometry. We found that gel phase liposomes were superior to fluid phase liposomes at activating DCs and significantly improved antigen processing and co-stimulation ability. The gel-phase liposomes (DSPC-PEG-FP) were the most efficient formulation to stimulate both peptide presentation and co-stimulation. Thus, the membrane phase-state of the liposome significantly impacts antigen processing and presentation in targeted DCs. Further, our data also show that the DSPC-PEG-FP formulation gave rise to a significant increase in peptide loading of MHC II, as indicated by the ratio between peptide and MHC II expression, while the increase in the total amount of MHC II on the cell surface was more modest.

An increased peptide occupancy can be related to a number of cellular changes. First of all, peptide loading and presentation is a highly dynamic process, involving a constant exchange of both MHC II with bound peptide and empty MHC II molecules at the cell surface. In this process, newly produced MHC II molecules, without peptide, are thought to be recirculated from the cell surface via the endosomal system and internalized by clathrin-dependent endocytosis before they bind processed peptides in late endosomes. The mature MHC II-peptide complexes can then be transported to the cell surface, where MARCH1 ubiquitinates the cytoplasmic tail of the MHC II 3 subunit, targeting the complexes for internalization and degradation. Upon dendritic cell maturation, transport of MHC II is modulated, leading to an increase in its half-life at the cell surface. This increase in half-life upon maturation is attributed to an overall decrease in endocytic activity upon maturation accompanied by a down-regulation of MARCH1, causing a decreased internalization of MHC II-peptide complexes. Hence the observed increase in occupancy could be related to a decreased recycling of MHC II from the cell surface, associated to ubiquitination via MARCH1. It is in this context interesting to note that that MARCH1-mediated ubiquitination is also a key process in the regulation of CD86 expression. We observed that FP-coupling to the liposome is not required to increase CD86 expression (DSPC-PEG-FP and DSPC-PEG+FP gave rise to very similar levels of CD86; FIG. 11C), while this aspect is central to maximal antigen presentation (DSPC-PEG-FP lead to significantly higher peptide occupancy than DSPC-PEG+FP; FIG. 11B). This suggests that a decreased MARCH1-mediated MHC II recycling is likely not the only mechanism to be involved.

An alternative explanation to the increased peptide expression could be related to a change in endosomal trafficking. It has, indeed, been shown that increased stiffness of capsules, approximately 4 μm in diameter, prolongs endocytic trafficking.

The increase in antigen processing and presentation, observed as enhanced peptide occupancy in MHC II and stronger co-stimulation, could also reflect a better uptake of this nanoparticle compared to the fluid phase DOPC-PEG-FP or free FP. This interpretation is supported by previous studies demonstrating a better uptake of rigid particles, an effect attributed to the higher bending and adhesion energies required to envelop deformed particles, making them more likely to remain trapped on the cell surface. In addition, the uptake process may be influenced by membrane fluidity, since the high ligand mobility characteristic for fluid phase is likely to have a major influence on multivalent attachment of the particle to the cell surface and the physiological processes responsible for uptake. An additional effect of membrane phase is that gel phase PC liposomes, unlike smooth fluid phase liposomes, have a facetted appearance and consist of gel phase domains connected by highly disordered grain boundaries in which FP-coupled PEG-lipids may collect at high local concentrations conducive to multivalent interactions. Finally, the uptake process could be affected by the stability of the coupling of the FP to the liposome. Indeed, an aspect which has been much less considered in the liposome drug delivery literature is that a change in membrane phase is also associated with changes in the membrane partitioning of lipid-bound antigen: fluid phase lipids are known to be retained less in lipid membrane as compared to gel phase lipids, an effect likely exacerbated by the hydrophilicity of the PEGylated FP-lipid conjugate. We can therefore not exclude that in the case of fluid phase DOPC-PEG-FP, a larger portion of the FP-lipid complexes is found in aqueous solution than with the gel phase DSPC-PEG-FP. This idea is consistent with observations showing that extraction of lipids from a disordered membrane has proven to require less force than extraction from an ordered one, which would imply that the forces that the cells exert on particles during the uptake process might be more likely to dislodge the FP-lipid complex from a fluid phase membrane than a gel phase one. The data showing very similar expression levels of the CD86 and cytokines, irrespective of whether FP was bound to DOPC-PEG or not (FIGS. 11 and 12), may indicate that it is possible that the fusion protein could have been more easily dislodged from the liposomes, a scenario likely to have a direct impact on the route and efficiency of uptake of the FP construct. Thus, this suggests that the differences in antigen presentation reported in this study between liposomes in a gel phase-state and their fluid phase counterparts could, at least in part, be attributed to a difference in liposome uptake. It is, however, noteworthy that also DSPC-PEG+FP causes statistically significant increases in antigen presentation and dendritic cell activation compared to free FP alone. Thus, differences in the ability to maintain FP coupled to the liposome are obviously important, but likely not the sole explanation for the adjuvant effect of DSPC-PEG liposomes.

The results, obtained using the in vitro model system, show that DSPC-PEG-FP has a potential to increase the immunogenicity of the fusion protein, suggesting that membrane fluidity, membrane rigidity and lipid partitioning are likely to significantly impact how carrier liposomes interact with, and are processed by, immune cells. The Example provides compelling evidence that gel phase liposomes can be an efficient means to further improve vaccine formulations that bring together vaccine antigens with adjuvant, protection and delivery aspects harnessed in the nanoparticle vector design. The mechanism(s) behind this effect may be related to a combination of stable particle integrity, increased uptake as well as speedy dendritic cell maturation and favorable conditions for peptide loading.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A nucleotide sequence encoding a fusion protein, wherein the nucleotide sequence comprises:
a first nucleotide sequence encoding a bacterial exotoxin; and
a second nucleotide sequence encoding a single chain antibody fragment (scFv) that specifically binds to a surface marker on antigen presenting cells.

2. The nucleotide sequence according to claim 1, wherein the bacterial exotoxin is selected from a group consisting of an A1 subunit of a bacterial enterotoxin and a pertussis toxin.

3. The nucleotide sequence according to claim 2, wherein the bacterial enterotoxin is selected from the group consisting of cholera toxin (CT) and Escherichia coli heat labile enterotoxin (LT).

4. The nucleotide sequence according to claim 3, wherein the bacterial enterotoxin is the A1 subunit of the bacterial enterotoxin is the A1 subunit of cholera toxin (CTA1).

5. The nucleotide sequence according to any of the claims 2 to 4, wherein the pertussis toxin is pertussis toxin subunit S1.

6. The nucleotide sequence according to any of the claims 1 to 5, wherein the second nucleotide sequence encodes a scFv that specifically binds to a surface marker on dendritic cells.

7. The nucleotide sequence according to claim 6, wherein the second nucleotide sequence encodes a scFv that specifically binds to a surface marker on conventional type 1 dendritic cells (cDC1s).

8. The nucleotide sequence according to claim 7, wherein the second nucleotide sequence encodes a scFv that specifically binds to a surface maker selected from the group consisting of cluster of differentiation 103 (CD103), c-type lectin domain family 9 member A (Clec9A), XCR1, lymphocyte antigen 75 (LY75), cell adhesion molecule 1 (CADM1), B- and T-lymphocyte attenuator (BTLA), dipeptidyl peptidase-4 (DPP4), and CD226.

9. The nucleotide sequence according to claim 8, wherein the second nucleotide sequence encodes a scFv that specifically binds to a surface maker selected from the group consisting of CD103, XCR1 and Clec9A.

10. The nucleotide sequence according to claim 6, wherein the second nucleotide sequence encodes a scFv that specifically binds to a surface marker on conventional dendritic cells (cDCs).

11. The nucleotide sequence according to claim 10, wherein the second nucleotide sequence encodes a scFv that specifically binds to thrombomodulin (TM).

12. The nucleotide sequence according to claim 6, wherein the second nucleotide sequence encodes a scFv that specifically binds to a surface marker on conventional type 2 dendritic cells (cDC2s).

13. The nucleotide sequence according to claim 12, wherein the second nucleotide sequence encodes a scFv that specifically binds to a surface maker selected from the group consisting of cluster of differentiation 1c (CD1c), CD11b, CD2, Fc epsilon RI (FCER1), signal regulatory protein alpha (SIRPA), leukocyte immunoglobulin-like receptor subfamily A member 2 (LILRA2), C-type lectin domain family 4 member A (Clec4A) and Clec10A.

14. The nucleotide sequence according to any of the claims 1 to 13, further comprising a third nucleotide sequence encoding at least one virus or bacterial epitope.

15. The nucleotide sequence according to claim 14, wherein the third nucleotide sequence encodes at least one ectodomain of matrix protein 2 (M2e) epitope of influenza A virus.

16. The nucleotide sequence according to claim 15, wherein the third nucleotide sequence encodes multiple M2e epitopes.

17. The nucleotide sequence according to any of the claims 14 to 16, wherein the nucleotide sequences comprises, from a 5′ end to a 3′ end, the first nucleotide sequence, the third nucleotide sequence and the second nucleotide sequence.

18. A nucleotide sequence according to any of the claims 1 to 17 for use as a medicament.

19. A nucleotide sequence according to any of the claims 1 to 13 for use as a vaccine adjuvant.

20. A nucleotide sequence according to any of the claims 14 to 17 for use as a vaccine.

21. An expression vector which comprises the nucleotide sequence according to any of the claims 1 to 17.

22. A cell comprising a nucleotide sequence according to any of the claims 1 to 17 and/or an expression vector according to claim 20.

23. A fusion protein comprising:
a bacterial exotoxin; and
a single chain antibody fragment (scFv) that specifically binds to a surface marker on antigen presenting cells.

24. The fusion protein according to claim 23, wherein the bacterial exotoxin is selected from a group consisting of an A1 subunit of a bacterial enterotoxin and a pertussis toxin.

25. The fusion protein according to claim 24, wherein the bacterial enterotoxin is selected from the group consisting of cholera toxin (CT) and Escherichia coli heat labile enterotoxin (LT).

26. The fusion protein according to claim 25, wherein the bacterial enterotoxin is the A1 subunit of cholera toxin (CTA1).

27. The fusion protein according to any of the claims 24 to 26, wherein the pertussis toxin is pertussis toxin subunit S1.

28. The fusion protein according to any of the claims 23 to 27, wherein the scFv specifically binds to a surface marker on dendritic cells.

29. The fusion protein according to claim 28, wherein the scFv specifically binds to a surface marker on conventional type 1 dendritic cells (cDC1s).

30. The fusion protein according to claim 29, wherein the scFv specifically binds to a surface maker selected from the group consisting of cluster of differentiation 103 (CD103), c-type lectin domain family 9 member A (CLEC9A), XCR1, lymphocyte antigen 75 (LY75), cell adhesion molecule 1 (CADM1), B- and T-lymphocyte attenuator (BTLA), dipeptidyl peptidase-4 (DPP4), and CD226.

31. The fusion protein according to claim 30, wherein the scFv specifically binds to a surface maker selected from the group consisting of CD103, XCR1 and CLEC9A.

32. The fusion protein according to claim 28, wherein the scFv specifically binds to a surface marker on conventional dendritic cells (cDCs).

33. The fusion protein according to claim 32, wherein the scFv specifically binds to thrombomodulin (TM).

34. The fusion protein according to claim 28, wherein the second nucleotide sequence encodes a scFv that specifically binds to a surface marker on conventional type 2 dendritic cells (cDC2s).

35. The fusion protein according to claim 34, wherein the scFv specifically binds to a surface maker selected from the group consisting of cluster of differentiation 1c (CD1c), CD11b, CD2, Fc epsilon RI (FCER1), signal regulatory protein alpha (SIRPA), leukocyte immunoglobulin-like receptor subfamily A member 2 (LILRA2), C-type lectin domain family 4 member A (CLEC4A) and CLEC10A.

36. The fusion protein according to any of the claims 23 to 35, further comprising at least one virus or bacterial epitope.

37. The fusion protein according to claim 36, wherein the at least one virus epitope is at least one ectodomain of matrix protein 2 (M2e) epitope of influenza A virus.

38. The fusion protein according to claim 37, wherein the fusion protein comprises multiple M2e epitopes.

39. The fusion protein according to any of the claims 36 to 38, wherein the fusion protein comprises, from an N-terminus to a C-terminus, the bacterial exotoxin, the at least one virus or bacterial epitope and the scFv.

40. An adjuvant composition comprising a fusion protein according to any of the claims 23 to 35 and a pharmaceutically acceptable carrier.

41. A vaccine composition comprising a fusion protein according to any of the claims 36 to 39 and a pharmaceutically acceptable carrier.

42. The composition according to claim 40 or 41, further comprising lipid nanoparticles (LNPs).

43. The composition according to claim 42, wherein the fusion protein is covalently coupled to a LNP, preferably the fusion protein is covalently coupled to the LNP by means of a linker selected from the group consisting of a polymer linker, poly(ethylene glycol) (PEG), a glycan, a polypeptide and an oligonucleotide.

44. A fusion protein according to any of the claims 23 to 39 for use as a medicament.

45. A fusion protein according to any of the claims 23 to 35 for use as a vaccine adjuvant.

46. A fusion protein according to any of the claims 36 to 39 for use as a vaccine.

47. A method of vaccinating a subject against a viral or bacterial infection or disease, the method comprising administering a vaccine composition according to any of the claims 41 to 43 to the subject.


SEQUENCE LISTING (43 sequences)

SEQ ID NO: 1 - DNA, 592 bp, Vibrio cholerae
atggatgataagttatatcgggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcgggatctacta

SEQ ID NO: 2 - DNA, 774 bp, Escherichia coli
atgaagaacatcaccttcatcttcttcatcctgctggccagccccctgtacgccaacggcgacaagctgtacagggccgacagcaggccccccgacgagatcaagcacagcggcggcctgatgcccaggggccacaacgagtacttcgacaggggcacccagatcaacatcaacctgtacgaccacgccaggggcacccagaccggcttcgtgaggtacgacgacggctacgtgagcaccagcctgagcctgaggagcgcccacctggccggccagagcatcctgagcggctacagcacctactacatctacgtgatcgccaccgcccccaacatgttcaacgtgaacgacgtgctgggcgtgtacagcccccacccctacgagcaggaggtgagcgccctgggcggcatcccctacagccagatctacggctggtacagggtgaacttcggcgtgatcgacgagaggctgcacaggaacagggagtacagggacaggtactacaggaacctgaacatcgcccccgccgaggacggctacaggctggccggcttcccccccgaccaccaggcctggagggaggagccctggatccaccacgccccccagggctgcggcaacagcagcaggaccatcaccggcgacacctgcaacgaggagacccagaacctgagcaccatctacctgaggaagtaccagagcaaggtgaagaggcagatcttcagcgactaccagagcgaggtggacatctacaacaggatcagggacgagctg

SEQ ID NO: 3 - DNA, 705 bp, Bordetella pertussis
gacgatcctcccgccaccgtataccgctatgactcccgcccgccggaggacgttttccagaacggattcacggcgtggggaaacaacgacaatgtgctcgaccatctgaccggacgttcctgccaggtcggcagcagcaacagcgctttcgtctccaccagcagcagccggcgctataccgaggtctatctcgaacatcgcatgcaggaagcggtcgaggccgaacgcgccggcaggggcaccggccacttcatcggctacatctacgaagtccgcgccgacaacaatttctacggcgccgccagctcgtacttcgaatacgtcgacacttatggcgacaatgccggccgtatcctcgccggcgcgctggccacctaccagagcgaatatctggcacaccggcgcattccgcccgaaaacatccgcagggtaacgcgggtctatcacaacggcatcaccggcgagaccacgaccacggagtattccaacgctcgctacgtcagccagcagactcgcgccaatcccaacccctacacatcgcgaaggtccgtagcgtcgatcgtcggcacattggtgcgcatggcgccggtgataggcgcttgcatggcgcggcaggccgaaagctccgaggccatggcagcctggtccgaacgcgccggcgaggcgatggttctcgtgtactacgaaagcatcgcgtattcgttc

SEQ ID NO: 4 - DNA, 765 bp, Artificial Sequence
  CD103-binding scFv
cccggggaggtgcagctggtggagtctgggggaggcttagtgcagcctggaaggtccctgaaactctcctgtgcagcctcaggattcactttcagtgactattacatggcctgggtccgccagtctccaaagaagggtctggagtgggtcgcatccattagttatgagggtactgacacttactatggagactccgtgaagggccgattcactatctccagagataatgcaaaaagcaccctgtacctgcaaatgaacagtctgaggtctgaggacacggccacttattattgtgcaagcttgaatggctataactatcgatactttgacttctggggcccaggaaccatggtcaccgtgtcctcaggtggaggcggttcaggcggaggtggctctggcggtggagggagcggtggagggagtaacatccagatgacccagtctccttcaatactgtctgcatctgtgggagacagagtcactctcagctgcaaagcaggtcagaatattaacaatttcttagcctggtatcagcaaaagcttggagaagctcccaaactcctgatatataatgcaaacagtttggaaacgggcatcccatcaaggttcagtggcagtggatctggcacagattacacactcaccatcagcagcctgcggcctgaagatgttgccacatatttctgccagcagtataacagttggtacacgtttggagctgggaccaagctggaactgaaacgggctgatgcggccgcaaagctt

SEQ ID NO: 5 - DNA, 778 bp, Artificial Sequence
  CD205-binding scFv
caggtcaagctgcaggagtcaggaggaggtttggtacagccggggggttctctgagactctcctgtgcagcttctggattcaccttcaatgatttctacatgaactggatccgccagcctccagggcaggcacctgagtggttgggtgttattagaaacaaaggtaatggttacacaacagaggtcaatacatctgtgaaggggcggttcaccatctccagagataatacccaaaacatcctctatcttcaaatgaacagcctgagagctgaggacaccgccatttactactgtgcaagaggcggtccttattactacagtggtgacgacgccccttactggggccaagggaccacggtcaccgtctcgagtggtggaggcggttcaggcggaggtggctctggcggtggagggagcggtggagggagtgcagacattgtgctcactcaatctccagcttccttagctgtatctctggggcagagggccaccatctcatgcagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagctttacacgttcggaggggggaccaagctggaaataaaacgggcggccgcataga

SEQ ID NO: 6 - protein, 16 aa, Influenza A virus
SLLTEVETPIRNEWGS

SEQ ID NO: 7 - protein, 23 aa, Influenza A virus
SLLTEVETPIRNEWGCRCNDSSD

SEQ ID NO: 8 - protein, 23 aa, Influenza A virus
MSLLTEVETPTRNEWECRCSDSS

SEQ ID NO: 9 - DNA, 48 bp, Influenza A virus
agcctgctgaccgaggtggagacccccatcaggaacgagtggggcagc

SEQ ID NO: 10 - DNA, 69 bp, Influenza A virus
agcctgctgaccgaggtggagacccccatcaggaacgagtggggctgcaggtgcaacgacagcagcgac

SEQ ID NO: 11 - DNA, 69 bp, Influenza A virus
atgagcctgctgaccgaggtggagacccccaccaggaacgagtgggagtgcaggtgcagcgacagcagc

SEQ ID NO: 12 - DNA, 1869 bp, Artificial Sequence
  Nt sequence encoding CTA1 - 3M2e - CD103- binding scFv
atgaaatttttggtgaacgtggccttggtgtttatggtggtgtacatttcgtacatttacgccgacccgtcgccggacgacaaattgtacagagccgactcgagaccgccggacgaaattaaacaatcgggcggcttgatgccgagaggccaatcggaatactttgacagaggcacgcaaatgaacattaacttgtacgaccacgccagaggcacgcaaacgggctttgtgagacacgacgacggctacgtgtcgacgtcgatttcgttgagatcggcccacttggtgggccaaacgattttgtcgggccactcgacgtactacatttacgtgattgccacggccccgaacatgtttaacgtgaacgacgtgttgggcgcctactcgccgcacccggacgaacaagaagtgtcggccttgggcggcattccgtactcgcaaatttacggctggtacagagtgcactttggcgtgttggacgaacaattgcacagaaacagaggctacagagacagatactactcgaacttggacattgccccggccgccgacggctacggcttggccggctttccgccggaacacagagcctggagagaagaaccgtggattcaccacgccccgccgggctgtggcaacgccccgagatcgtcggggaattcgttgttgacggaagtggaaacgccgattagaaacgaatggggctcgagatcgaacgactcgtcgttgttgacggaagtggaaacgccgattagaaacgaatggggctcgagatcgaacgactcgtcgttgttgacggaagtggaaacgccgattagaaacgaatggggctcgagatcgaacgactcgtcggacggtttaattaagtttaaaccatatgaagccagcttcgaggcccagggcgccctggccaacatcgccgtggacaaggccgccagcttcgaggcccagggcgccctggccaacatcgccgtggacaaggccgccagcttcgaggcccagggcgccctggccaacatcgccgtggacaaggccagtttaaaccccgggagcggggaggtgcagctggtggagtctgggggaggcttagtgcagcctggaaggtccctgaaactctcctgtgcagcctcaggattcactttcagtgactattacatggcctgggtccgccagtctccaaagaagggtctggagtgggtcgcatccattagttatgagggtactgacacttactatggagactccgtgaagggccgattcactatctccagagataatgcaaaaagcaccctgtacctgcaaatgaacagtctgaggtctgaggacacggccacttattattgtgcaagcttgaatggctataactatcgatactttgacttctggggcccaggaaccatggtcaccgtgtcctcaggtggaggcggttcaggcggaggtggctctggcggtggagggagcggtggagggagtaacatccagatgacccagtctccttcaatactgtctgcatctgtgggagacagagtcactctcagctgcaaagcaggtcagaatattaacaattttttagcctggtatcagcaaaagcttggagaagctcccaaactcctgatatataatgcaaacagtttggaaacgggcatcccatcaaggttcagtggcagtggatttggcacagattacacactcaccatcagcagcctgcggcctgaagatgttgccacatatttttgccagcagtataacagttggtacacgtttggaggtgggaccaagctggaaatgaaacgggctgatgcggccgcatattcgaagagctccgactacaaagacgacgacgacaaacaccaccaccaccaccactag

SEQ ID NO: 13 - protein, 197 aa, Vibrio cholerae
MDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSGST

SEQ ID NO: 14 - protein, 212 aa, Escherichia coli
MKNITFIFFILLASPLYANGDKLYRADSRPPDEIKHSGGLMPRGHNEYFDRGTQININLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRTI

SEQ ID NO: 15 - protein, 235 aa, Bordetella pertussis
DDPPATVYRYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSEYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 16 - protein, 253 aa, Artificial Sequence
  CD103-binding scFv
PGEVQLVESGGGLVQPGRSLKLSCAASGFTFSDYYMAWVRQSPKKGLEWVASISYEGTDTYYGDSVKGRFTISRDNAKSTLYLQMNSLRSEDTATYYCASLNGYNYRYFDFWGPGTMVTVSSGGGGSGGGGSGGGGSGGGSNIQMTQSPSILSASVGDRVTLSCKAGQNINNFLAWYQQKLGEAPKLLIYNANSLETGIPSRFSGSGSGTDYTLTISSLRPEDVATYFCQQYNSWYTFGAGTKLELKRADAAA

SEQ ID NO: 17 - protein, 22 aa, Artificial Sequence
  SARS-CoV-2
FGAGAALQIPFAMQMAYRFNGI

SEQ ID NO: 18 - protein, 18 aa, Artificial Sequence
  SARS-CoV-2
QLIRAAEIRASANLAATK

SEQ ID NO: 19 - protein, 9 aa, Artificial Sequence
  SARS-CoV-2
MMISAGFSL

SEQ ID NO: 20 - DNA, 60 bp, Artificial Sequence
  SARS-CoV-2
tttggcgcgggcgcggcgctgcagattccgtttgcgatgcagatggcgtatcgctttaac

SEQ ID NO: 21 - DNA, 54 bp, Artificial Sequence
  SARS-CoV-2
cagctgattcgcgcggcggaaattcgcgcgagcgcgaacctggcggcgaccaaa

SEQ ID NO: 22 - DNA, 27 bp, Artificial Sequence
  SARS-CoV-2
atgatgattagcgcgggctttagcctg

SEQ ID NO: 23 - DNA, 468 bp, Artificial Sequence
  Amplified cDNA from M290 hybridoma
gatagatacagttggtgcagcatcagcccgtttcatttccagcttggtcccacctccaaacgtgtaccaactgttatactgctggcaaaaatatgtggcaacatcttcaggccgcaggctgctgatggtgagtgtgtaatctgtgccaaatccactgccactgaaccttgatgggatgcccgtttccaaactgtttgcattatatatcaggagtttgggagcttctccaagcttttgctgataccaggctaaaaaattgttaatattctgacctgctttgcagctgagagtgactctgtctcccacagatgcagacagtattgaaggagactgggtcatctggatgttacatctcagggctgggagccagagcaacaccagccctaagagttgaactggagccatcatgactggcctgtgtcctgtctgagactgaataccaaagcctgccgggggggggggggaaca

SEQ ID NO: 24 - DNA, 564 bp, Artificial Sequence
  Amplified cDNA from M290 hybridoma
taagtacttttgagagcagttccaggagccagtggatagacagatggggctgttgtttcagctgaggacacggtgaccatggttcctgggccccagaagtcaaagtatcgatagttatagccattcaagcttgcacaataataagtggccgtgtcctcagacctcagactgttcatttgcaggtacagggtgctttttgcattatctctggagatagtgaatcggcccttcacggagtctccatagtaagtgtcagtaccctcataactaatggatgcgacccactccagacccttctttggagactggcggacccaggccatgtaatagtcactgaaagtgaatcctgaggctgcacaggagagtttcagggaccttccaggctgcactaagcctcccccagactccaccagctgcacctcacactggacaccttttatgaaaaggacaaggaaaaccaagctgagcctggtgtccatggtgagtagtctgtgcagtgctgcagtactgattactgaatgggagagcctcagagtccaggactgggctcctgcgtccgagggg

SEQ ID NO: 25 - DNA, 43 bp, Artificial Sequence
  M290 VH forward primer
ggcggatagatctactagtcccggggaggtgcagctggtggag

SEQ ID NO: 26 - DNA, 33 bp, Artificial Sequence
  M290 VH reverse primer
tgaggacacggtgaccatggttcctgggcccca

SEQ ID NO: 27 - DNA, 33 bp, Artificial Sequence
  M290 VL forward primer
aacatccagatgacccagtctccttcaatactg

SEQ ID NO: 28 - DNA, 42 bp, Artificial Sequence
  M290 VL reverse primer
gccgccaagcttctatgcggccgcgaaatgaaacgggctgat

SEQ ID NO: 29 - DNA, 366 bp, Artificial Sequence
  VH sequence
cccggggaggtgcagctggtggagtctgggggaggcttagtgcagcctggaaggtccctgaaactctcctgtgcagcctcaggattcactttcagtgactattacatggcctgggtccgccagtctccaaagaagggtctggagtgggtcgcatccattagttatgagggtactgacacttactatggagactccgtgaagggccgattcactatctccagagataatgcaaaaagcaccctgtacctgcaaatgaacagtctgaggtctgaggacacggccacttattattgtgcaagcttgaatggctataactatcgatactttgacttctggggcccaggaaccatggtcaccgtgtcctca

SEQ ID NO: 30 - DNA, 351 bp, Artificial Sequence
  VL sequence
aacatccagatgacccagtctccttcaatactgtctgcatctgtgggagacagagtcactctcagctgcaaagcaggtcagaatattaacaattttttagcctggtatcagcaaaagcttggagaagctcccaaactcctgatatataatgcaaacagtttggaaacgggcatcccatcaaggttcagtggcagtggatttggcacagattacacactcaccatcagcagcctgcggcctgaagatgttgccacatatttttgccagcagtataacagttggtacacgtttggaggtgggaccaagctggaaatgaaacgggctgatgcggccgcatagaagcttggcggc

SEQ ID NO: 31 - DNA, 87 bp, Artificial Sequence
  GS-M290 forward primer
gtcaccgtgtcctcaggtggaggcggttcaggcggaggtggctctggcggtggagggagcggtggagggagtaacatccagatgacc

SEQ ID NO: 32 - DNA, 87 bp, Artificial Sequence
  GS-M290 reverse primer
ggtcatctggatgttactccctccaccgctccctccaccgccagagccacctccgcctgaaccgcctccacctgaggacacggtgac

SEQ ID NO: 33 - DNA, 793 bp, Artificial Sequence
  CD103 scFv
ggcggatagatctactagtcccggggaggtgcagctggtggagtctgggggaggcttagtgcagcctggaaggtccctgaaactctcctgtgcagcctcaggattcactttcagtgactattacatggcctgggtccgccagtctccaaagaagggtctggagtgggtcgcatccattagttatgagggtactgacacttactatggagactccgtgaagggccgattcactatctccagagataatgcaaaaagcaccctgtacctgcaaatgaacagtctgaggtctgaggacacggccacttattattgtgcaagcttgaatggctataactatcgatactttgacttctggggcccaggaaccatggtcaccgtgtcctcaggtggaggcggttcaggcggaggtggctctggcggtggagggagcggtggagggagtaacatccagatgacccagtctccttcaatactgtctgcatctgtgggagacagagtcactctcagctgcaaagcaggtcagaatattaacaattttttagcctggtatcagcaaaagcttggagaagctcccaaactcctgatatataatgcaaacagtttggaaacgggcatcccatcaaggttcagtggcagtggatttggcacagattacacactcaccatcagcagcctgcggcctgaagatgttgccacatatttttgccagcagtataacagttggtacacgtttggaggtgggaccaagctggaaatgaaacgggctgatgcggccgcatagaagcttggcggc

SEQ ID NO: 34 - DNA, 604 bp, Vibrio cholerae
ggtctccccatgaaaaatgatgataagttatatcgcgcagattctcgtccgcctgatgaaattaaacagagcggtggtctgatgcctcgtggtcagagcgaatattttgatcgtggcacccagatgaacatcaacctgtatgatcatgcacgtggtacacagaccggttttgttcgtcatgatgatggttatgttagcaccagcattagcctgcgtagcgcacatctggttggtcagaccattctgagcggtcatagcacctattatatctatgttattgcaaccgcaccgaatatgtttaacgttaatgatgttctgggtgcctatagtccgcatccggatgaacaagaggttagcgcactgggtggtattccgtatagccagatttatggttggtatcgtgttcattttggtgtgctggatgaacagctgcatcgtaatcgtggttatcgtgatcgttattatagcaacctggatattgcaccggcagcagatggttatggtctggcaggttttccgcctgaacatcgtgcatggcgtgaagaaccgtggattcatcatgcaccgcctggtgcaggtaatgcaccgcgtagctctcgagacc

SEQ ID NO: 35 - DNA, 444 bp, Artificial Sequence
  3M2e-3Ea-SIIN-p323
ggtctccctctagcctgctgaccgaagttgaaaccccgattcgtaatgaatggggtagccgtagcaatgatagcagtctgttaacggaagtggaaacaccgatccgcaacgagtggggtagtcgttcaaatgatagctcactgctgacagaggtggaaacgcctataagaaacgaatggggttcacgtagtaatgattcaagcgcaagctttgaagcacagggtgcactggcaaatattgcagttgataaagcagccagttttgaggcccaaggtgccctggccaacattgccgtggacaaagcagcgtcattcgaggcgcagggtgcattagccaatatcgccgtggataaggcaagcattatcaactttgagaaactgaaaattagccaggcagttcatgcagcacatgccgaaattaatgaagcaggtcgagcacgagacc

SEQ ID NO: 36 - DNA, 836 bp, Artificial Sequence
  CD103-binding scFv with FLAG and His tag
ggtctccagcaagtccgggtgaagttcagctggttgaaagcggtggtggtctggttcagcctggtcgtagcctgaaactgagctgtgcagcaagcggttttacctttagcgattattacatggcatgggttcgtcagagcccgaaaaaaggtctggaatgggttgcaagcattagctatgaaggcaccgatacctattatggtgatagcgttaaaggtcgctttaccattagccgtgataatgcaaaaagcaccctgtacctgcagatgaatagcctgcgtagcgaagataccgcaacctattattgtgcaagcctgaatggttataactaccgctattttgatttttggggtccgggtacaatggttaccgttagctcaggtggtggtggtagcggtggcggtggttctggtggtggcggatcaggtggcggtagcaatattcagatgacccagagtccgagcattctgagcgcaagcgttggtgatcgtgttaccctgagctgtaaagcaggtcagaatattaacaatttcctggcatggtatcagcagaaactgggtgaagcaccgaaactgctgatttataacgcaaatagcctggaaaccggtattccgagccgttttagcggtagcggttctggcaccgattataccctgaccattagcagcctgcgtccggaagatgttgccacctatttttgtcagcagtataatagctggtatacctttggtgccggtacaaaactggaactgaaacgtgcagatgcagcagccaaactggattataaagatgatgatgataagcaccatcatcaccaccactaataagccgagacc

SEQ ID NO: 37 - DNA, 4243 bp, Artificial Sequence
  E. coli expression vector
ggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggagacctaataactgcagtaataaggtctctaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtgggatctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatggtgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgtt

SEQ ID NO: 38 - DNA, 1519 bp, Artificial Sequence
  CTA1-SIINFEKL(I)-P232(II)-CD103 scFv-FLAG tag- His Tag (
atcacctgcataccatggatgataagttatatcgggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcgggatctactagtagtataatcaactttgaaaaactgatctcccaggctgttcacgctgctcacgctgaaatcaacgaagctggtcgtgccagatctcccggggaggtgcagctggtggagtctgggggaggcttagtgcagcctggaaggtccctgaaactctcctgtgcagcctcaggattcactttcagtgactattacatggcctgggtccgccagtctccaaagaagggtctggagtgggtcgcatccattagttatgagggtactgacacttactatggagactccgtgaagggccgattcactatctccagagataatgcaaaaagcaccctgtacctgcaaatgaacagtctgaggtctgaggacacggccacttattattgtgcaagcttgaatggctataactatcgatactttgacttctggggcccaggaaccatggtcaccgtgtcctcaggtggaggcggttcaggcggaggtggctctggcggtggagggagcggtggagggagtaacatccagatgacccagtctccttcaatactgtctgcatctgtgggagacagagtcactctcagctgcaaagcaggtcagaatattaacaatttcttagcctggtatcagcaaaagcttggagaagctcccaaactcctgatatataatgcaaacagtttggaaacgggcatcccatcaaggttcagtggcagtggatctggcacagattacacactcaccatcagcagcctgcggcctgaagatgttgccacatatttctgccagcagtataacagttggtacacgtttggagctgggaccaagctggaactgaaacgggctgatgcggccgcaaagcttgattacaaggatgacgatgacaagcatcaccatcatcaccattagagcttcgcaggtgcttc

SEQ ID NO: 39 - DNA, 1499 bp, Artificial Sequence
  CTA1-P232(II)-CD103 scFv-FLAG tag-His Tag
atcacctgcataccatggatggatgataagttatataaggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcgggatctactagtatctcccaggctgttcacgctgctcacgctgaaatcaacgaagctggtcgtgccagatctcccggggaggtgcagctggtggagtctgggggaggcttagtgcagcctggaaggtccctgaaactctcctgtgcagcctcaggattcactttcagtgactattacatggcctgggtccgccagtctccaaagaagggtctggagtgggtcgcatccattagttatgagggtactgacacttactatggagactccgtgaagggccgattcactatctccagagataatgcaaaaagcaccctgtacctgcaaatgaacagtctgaggtctgaggacacggccacttattattgtgcaagcttgaatggctataactatcgatactttgacttctggggcccaggaaccatggtcaccgtgtcctcaggtggaggcggttcaggcggaggtggctctggcggtggagggagcggtggagggagtaacatccagatgacccagtctccttcaatactgtctgcatctgtgggagacagagtcactctcagctgcaaagcaggtcagaatattaacaattttttagcctggtatcagcaaaagcttggagaagctcccaaactcctgatatataatgcaaacagtttggaaacgggcatcccatcaaggttcagtggcagtggatttggcacagattacacactcaccatcagcagcctgcggcctgaagatgttgccacatatttttgccagcagtataacagttggtacacgtttggaggtgggaccaagctggaaatgaaacgggctgatgcggccgcaaagcttgattacaaggatgacgatgacaagcatcaccatcatcaccattagagcttcgcaggtgcttc

SEQ ID NO: 40 - DNA, 1499 bp, Artificial Sequence; identical to SEQ ID NO: 39
  CTA1(R7k)-P323- CD103 scFv-FLAG tag-His Tag

SEQ ID NO: 41 - DNA, 4151 bp, Artificial Sequence
  pSY-AaRI
ctccaaaaggagcctttaattgtatcggtttatcagcttgctttcgaggtgaatttcgacctctagaccacgcttggctgcaggtcgacggatctcgatcccgcgaaattaatacgactcactatagggagaccacaacggtttccctctagaaataattttgtttaactttaagaaggagatataccatggatggcaggtgctataagttaatcatcaccacctgccattagagcttctcaaataagatggtcccatagtctgtatccaaataatgaatcttcgggtgtttccctttagctaagcacagatccggctgctaacaaagcccgaaaggaagctgagttggctgctgccaccgctgagcaataactagcataaccccttggggcctctaaacgggtcttgaggggttttttgctgaaaggaggaactatatccggatgggaattccccgcgcgcgatgccctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggcgaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgagcccgcctaatgagcgggcttttttttagcccgcctaatgagcgggcttttttttcgaaagcaaattcgacccatcgcgcgcggggagtcaactcagcaaaagttcgatttattcaacaaagccacgttgtgtctcaaaatctctgatgttacattgcacaagataaaaatatatcatcatgaacaataaaactgtctgcttacataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagatcaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtgatctgcatccgcttacagacaagctgtgaccgtctccgggagctgcatgtgtcagaggttttcaccgtcatcaccgaaacgcgcgaggcagagcttggaattgcgaataataattttttcacgttgaaaatctccaaaaaaaaaggctccaaaaggagcctttaattgtatcggtttatcagcttgctttcgaggtgaattagcttggaattgcgaataataattttttcacgttgaaaatctccaaaaaaaaagg

SEQ ID NO: 42 - DNA, 1887 bp, Artificial Sequence
  mSS-CTA1-3(M2e)-3(Ea)-CD103 scFv- FLAG tag- His Tag
gcgcggatccatgaaatttttggtgaacgtggccttggtgtttatggtggtgtacatttcgtacatttacgccgacccgtcgccggacgacaaattgtacagagccgactcgagaccgccggacgaaattaaacaatcgggcggcttgatgccgagaggccaatcggaatactttgacagaggcacgcaaatgaacattaacttgtacgaccacgccagaggcacgcaaacgggctttgtgagacacgacgacggctacgtgtcgacgtcgatttcgttgagatcggcccacttggtgggccaaacgattttgtcgggccactcgacgtactacatttacgtgattgccacggccccgaacatgtttaacgtgaacgacgtgttgggcgcctactcgccgcacccggacgaacaagaagtgtcggccttgggcggcattccgtactcgcaaatttacggctggtacagagtgcactttggcgtgttggacgaacaattgcacagaaacagaggctacagagacagatactactcgaacttggacattgccccggccgccgacggctacggcttggccggctttccgccggaacacagagcctggagagaagaaccgtggattcaccacgccccgccgggctgtggcaacgccccgagatcgtcggggaattcgttgttgacggaagtggaaacgccgattagaaacgaatggggctcgagatcgaacgactcgtcgttgttgacggaagtggaaacgccgattagaaacgaatggggctcgagatcgaacgactcgtcgttgttgacggaagtggaaacgccgattagaaacgaatggggctcgagatcgaacgactcgtcggacggtttaattaagtttaaaccatatgaagccagcttcgaggcccagggcgccctggccaacatcgccgtggacaaggccgccagcttcgaggcccagggcgccctggccaacatcgccgtggacaaggccgccagcttcgaggcccagggcgccctggccaacatcgccgtggacaaggccagtttaaaccccgggagcggggaggtgcagctggtggagtctgggggaggcttagtgcagcctggaaggtccctgaaactctcctgtgcagcctcaggattcactttcagtgactattacatggcctgggtccgccagtctccaaagaagggtctggagtgggtcgcatccattagttatgagggtactgacacttactatggagactccgtgaagggccgattcactatctccagagataatgcaaaaagcaccctgtacctgcaaatgaacagtctgaggtctgaggacacggccacttattattgtgcaagcttgaatggctataactatcgatactttgacttctggggcccaggaaccatggtcaccgtgtcctcaggtggaggcggttcaggcggaggtggctctggcggtggagggagcggtggagggagtaacatccagatgacccagtctccttcaatactgtctgcatctgtgggagacagagtcactctcagctgcaaagcaggtcagaatattaacaattttttagcctggtatcagcaaaagcttggagaagctcccaaactcctgatatataatgcaaacagtttggaaacgggcatcccatcaaggttcagtggcagtggatttggcacagattacacactcaccatcagcagcctgcggcctgaagatgttgccacatatttttgccagcagtataacagttggtacacgtttggaggtgggaccaagctggaaatgaaacgggctgatgcggccgcatattcgaagagctccgactacaaagacgacgacgacaaacaccaccaccaccaccactagggtaccaa

SEQ ID NO: 43 - protein, 601 aa, Artificial Sequence
  CTA1 - 3M2e - 3Ea - CD103-binding scFv - FLAG tag - His tag
DPSPDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSGNSLLTEVETPIRNEWGSRSNDSSLLTEVETPIRNEWGSRSNDSSLLTEVETPIRNEWGSRSNDSSDGLIKFKPYEASFEAQGALANIAVDKAASFEAQGALANIAVDKAASFEAQGALANIAVDKASLNPGSGEVQLVESGGGLVQPGRSLKLSCAASGFTFSDYYMAWVRQSPKKGLEWVASISYEGTDTYYGDSVKGRFTISRDNAKSTLYLQMNSLRSEDTATYYCASLNGYNYRYFDFWGPGTMVTVSSGGGGSGGGGSGGGGSGGGSNIQMTQSPSILSASVGDRVTLSCKAGQNINNFLAWYQQKLGEAPKLLIYNANSLETGIPSRFSGSGFGTDYTLTISSLRPEDVATYFCQQYNSWYTFGGGTKLEMKRADAAAYSKSSDYKDDDDKHHHHHH